(19) United States
(12) Patent Application Publication
(10) Pub. No.: US 2026/0234229 A1
(43) Pub. Date: Aug. 13, 2026

(54) PATHOLOGIC TDP-43 AS A BIOMARKER FOR THE DIAGNOSIS OF TDP-43 PROTEINOPATHY

(71) Applicant: UNIVERSITY OF UTAH RESEARCH FOUNDATION, Salt Lake City, UT (US)

(72) Inventor: Qinwen MAO, Salt Lake City, UT (US)

(21) Appl. No.: 19/366,344

(22) Filed: Oct. 22, 2025

Related U.S. Application Data
(63) Continuation-in-part of application No. 17/937,339, filed on Sep. 30, 2022, now Pat. No. 12,625,149.
(60) Provisional application No. 63/251,383, filed on Oct. 1, 2021.
(60) Provisional application No. 63/252,353, filed on Oct. 5, 2021.

(51) Int. Cl. C07K 16/18; G01N 33/68

(52) U.S. Cl. CPC C07K 16/18; G01N 33/6896; C07K 2317/34; C07K 2317/565; C07K 2317/92; G01N 2440/14; G01N 2800/28

(57) ABSTRACT

Disclosed herein are antibodies or antigen-binding fragments thereof and compositions comprising the same. Also disclosed are methods of detecting TAR DNA-binding protein 43 (TDP-43) in a biological sample, diagnosing a neurodegenerative disease in a subject, and selecting whether to enroll a subject in a clinical trial for frontotemporal lobar degeneration with TDP-43 inclusions (FTLD-TDP) using the antibodies or antigen-binding fragments thereof described herein. In addition, disclosed herein are immunoassay kits for selectively detecting TDP-43 in a biological sample.

### CROSS-REFERENCE TO RELATED APPLICATIONS

This application is a continuation-in-part of U.S. patent application Ser. No. 17/937,339, filed Sep. 30, 2022, which claims priority to U.S. Provisional Patent Application No. 63/251,383, filed Oct. 1, 2021, and U.S. Provisional Patent Application No. 63/252,353 filed Oct. 5, 2021, each of which is incorporated by reference herein in its entirety.

### REFERENCE TO SEQUENCE LISTING

This application was filed with a Sequence Listing XML in ST.26 XML format in accordance with 37 C.F.R. § 1.831. The Sequence Listing XML file submitted in the USPTO Patent Center, “026389-9326-US04_sequence_listing_xml_22 Oct. 2025.xml,” was created on Oct. 22, 2025, contains 85 sequences, has a file size of 89.1 kilobytes (91,335 bytes), and is incorporated by reference in its entirety into the specification.

### TECHNICAL FIELD

This disclosure relates to antibodies or antigen-binding fragments thereof and compositions comprising the same. The disclosure further relates to methods of detecting TAR DNA-binding protein 43 (TDP-43) in a biological sample, diagnosing a neurodegenerative disease in a subject, and selecting whether to enroll a subject in a clinical trial for frontotemporal lobar degeneration with TDP-43 inclusions (FTLD-TDP) using the antibodies or antigen-binding fragments thereof described herein. In addition, the disclosure relates to immunoassay kits for selectively detecting TDP-43 in a biological sample.

### INTRODUCTION

Populations are aging and neurodegenerative disease is becoming a growing and intractable health challenge. While Alzheimer's disease (AD) is the most common cause of dementia, frontotemporal dementia (FTD) is the second most common in people under 65. FTD is a heterogeneous neurodegenerative disease characterized by progressive behavior and/or language impairments that include a range of clinical subtypes, e.g., behavioral variant FTD, progressive non-fluent aphasia, and semantic dementia. Pathologically, most patients have frontotemporal lobar degeneration (FTLD). About half of those patients show an accumulation of hyperphosphorylated tau proteins (in a subtype called FTLD-tau), while most others show an accumulation of the transactive DNA-binding protein TDP-43 (the subtype FTLD-TDP). However, these clinical conditions may also be caused by unusual presentations of AD. Because different underlying pathologies require specific therapeutic interventions, robust biomarkers are urgently needed to correctly select appropriate drugs for individual patients based on their specific underlying molecular pathology (e.g., FTLD-tau, FTLD-TDP, or AD). Though signature cerebrospinal fluid (CSF) markers exist for AD (low Aβ42 together with high T-tau and P-tau), currently there are no reliable structural/functional imaging methodologies nor CSF/serum liquid biomarker-based methodologies available for diagnosing FTLD-tau or FTLD-TDP.

Thus, there is a need for robust plasma biomarkers for diagnosing FTLD-TDP and highly sensitive and specific immunoassays for the detection and quantification of the biomarker.

### SUMMARY

One embodiment described herein is an isolated antibody or antigen-binding fragment thereof capable of binding to a TAR DNA-binding protein 43 (TDP-43) protein or fragment thereof, the antibody or antigen-binding fragment thereof comprising an immunoglobulin heavy chain (HC) variable domain sequence and an immunoglobulin light chain (LC) variable domain sequence, wherein: the immunoglobulin HC variable domain sequence comprises: (i) an HC CDR1 comprising an amino acid sequence having at least 95-99% identity to SEQ ID NO: 10, (ii) an HC CDR2 comprising an amino acid sequence having at least 95-99% identity to SEQ ID NO: 12, and (iii) an HC CDR3 comprising an amino acid sequence having at least 95-99% identity to SEQ ID NO: 14; and the immunoglobulin LC variable domain sequence comprises: (i) an LC CDR1 comprising an amino acid sequence having at least 95-99% identity to SEQ ID NO: 20, (ii) an LC CDR2 comprising an amino acid sequence having at least 95-99% identity to SEQ ID NO: 22, and (iii) an LC CDR3 comprising an amino acid sequence having at least 95-99% identity to SEQ ID NO: 24; the immunoglobulin HC variable domain sequence comprises: (i) an HC CDR1 comprising an amino acid sequence having at least 95-99% identity to SEQ ID NO: 30, (ii) an HC CDR2 comprising an amino acid sequence having at least 95-99% identity to SEQ ID NO: 32, and (iii) an HC CDR3 comprising an amino acid sequence having at least 95-99% identity to SEQ ID NO: 34; and the immunoglobulin LC variable domain sequence comprises: (i) an LC CDR1 comprising an amino acid sequence having at least 95-99% identity to SEQ ID NO: 40, (ii) an LC

CDR2 comprising an amino acid sequence having at least 95-99% identity to SEQ ID NO: 42, and (iii) an LC CDR3 comprising an amino acid sequence having at least 95-99% identity to SEQ ID NO: 44; the immunoglobulin HC variable domain sequence comprises: (i) an HC CDR1 comprising an amino acid sequence having at least 95-99% identity to SEQ ID NO: 50, (ii) an HC CDR2 comprising an amino acid sequence having at least 95-99% identity to SEQ ID NO: 52, and (iii) an HC CDR3 comprising an amino acid sequence having at least 95-99% identity to SEQ ID NO: 54; and the immunoglobulin LC variable domain sequence comprises: (i) an LC CDR1 comprising an amino acid sequence having at least 95-99% identity to SEQ ID NO: 60, (ii) an LC CDR2 comprising an amino acid sequence having at least 95-99% identity to SEQ ID NO: 62, and (iii) an LC CDR3 comprising an amino acid sequence having at least 95-99% identity to SEQ ID NO: 64; or the immunoglobulin HC variable domain sequence comprises: (i) an HC CDR1 comprising an amino acid sequence having at least 95-99% identity to SEQ ID NO: 70, (ii) an HC CDR2 comprising an amino acid sequence having at least 95-99% identity to SEQ ID NO: 72, and (iii) an HC CDR3 comprising an amino acid sequence having at least 95-99% identity to SEQ ID NO: 74; and the immunoglobulin LC variable domain sequence comprises: (i) an LC CDR1 comprising an amino acid sequence having at least 95-99% identity to SEQ ID NO: 80, (ii) an LC CDR2 comprising an amino acid sequence having at least 95-99% identity to SEQ ID NO: 82, and (iii) an LC CDR3 comprising an amino acid sequence having at least 95-99% identity to SEQ ID NO: 84. In one aspect, the immunoglobulin HC variable domain sequence comprises: (i) an HC CDR1 comprising the amino acid sequence of SEQ ID NO: 10, (ii) an HC CDR2 comprising the amino acid sequence of SEQ ID NO: 12, and (iii) an HC CDR3 comprising the amino acid sequence of SEQ ID NO: 14; and the immunoglobulin LC variable domain sequence comprises: (i) an LC CDR1 comprising the amino acid sequence of SEQ ID NO: 20, (ii) an LC CDR2 comprising the amino acid sequence of SEQ ID NO: 22, and (iii) an LC CDR3 comprising the amino acid sequence of SEQ ID NO: 24; the immunoglobulin HC variable domain sequence comprises: (i) an HC CDR1 comprising the amino acid sequence of SEQ ID NO: 30, (ii) an HC CDR2 comprising the amino acid sequence of SEQ ID NO: 32, and (iii) an HC CDR3 comprising the amino acid sequence of SEQ ID NO: 34; and the immunoglobulin LC variable domain sequence comprises: (i) an LC CDR1 comprising the amino acid sequence of SEQ ID NO: 40, (ii) an LC CDR2 comprising the amino acid sequence of SEQ ID NO: 42, and (iii) an LC CDR3 comprising the amino acid sequence of SEQ ID NO: 44; the immunoglobulin HC variable domain sequence comprises: (i) an HC CDR1 comprising the amino acid sequence of SEQ ID NO: 50, (ii) an HC CDR2 comprising the amino acid sequence of SEQ ID NO: 52, and (iii) an HC CDR3 comprising the amino acid sequence of SEQ ID NO: 54; and the immunoglobulin LC variable domain sequence comprises: (i) an LC CDR1 comprising the amino acid sequence of SEQ ID NO: 60, (ii) an LC CDR2 comprising the amino acid sequence of SEQ ID NO: 62, and (iii) an LC CDR3 comprising the amino acid sequence of SEQ ID NO: 64; or the immunoglobulin HC variable domain sequence comprises: (i) an HC CDR1 comprising the amino acid sequence of SEQ ID NO: 70, (ii) an HC CDR2 comprising the amino acid sequence of SEQ ID NO: 72, and (iii) an HC CDR3 comprising the amino acid sequence of SEQ ID NO: 74; and the immunoglobulin LC variable domain sequence comprises: (i) an LC CDR1 comprising the amino acid sequence of SEQ ID NO: 80, (ii) an LC CDR2 comprising the amino acid sequence of SEQ ID NO: 82, and (iii) an LC CDR3 comprising the amino acid sequence of SEQ ID NO: 84. In another aspect, the immunoglobulin HC variable domain sequence comprises an amino acid sequence having at least 95-99% identity to SEQ ID NO: 8, and the immunoglobulin LC variable domain sequence comprises an amino acid sequence having at least 95-99% identity to SEQ ID NO: 18; the immunoglobulin HC variable domain sequence comprises an amino acid sequence having at least 95-99% identity to SEQ ID NO: 28, and the immunoglobulin LC variable domain sequence comprises an amino acid sequence having at least 95-99% identity to SEQ ID NO: 38; the immunoglobulin HC variable domain sequence comprises an amino acid sequence having at least 95-99% identity to SEQ ID NO: 48, and the immunoglobulin LC variable domain sequence comprises an amino acid sequence having at least 95-99% identity to SEQ ID NO: 58; or the immunoglobulin HC variable domain sequence comprises an amino acid sequence having at least 95-99% identity to SEQ ID NO: 68, and the immunoglobulin LC variable domain sequence comprises an amino acid sequence having at least 95-99% identity to SEQ ID NO: 78. In another aspect, the immunoglobulin HC variable domain sequence comprises the amino acid sequence of SEQ ID NO: 8, and the immunoglobulin LC variable domain sequence comprises the amino acid sequence of SEQ ID NO: 18; the immunoglobulin HC variable domain sequence comprises the amino acid sequence of SEQ ID NO: 28, and the immunoglobulin LC variable domain sequence comprises the amino acid sequence of SEQ ID NO: 38; the immunoglobulin HC variable domain sequence comprises the amino acid sequence of SEQ ID NO: 48, and the immunoglobulin LC variable domain sequence comprises the amino acid sequence of SEQ ID NO: 58; or the immunoglobulin HC variable domain sequence comprises the amino acid sequence of SEQ ID NO: 68, and the immunoglobulin LC variable domain sequence comprises the amino acid sequence of SEQ ID NO: 78. In another aspect, the immunoglobulin HC variable domain sequence is encoded by a nucleic acid comprising the nucleotide sequence of SEQ ID NO: 7, and the immunoglobulin LC variable domain sequence is encoded by a nucleic acid comprising the nucleotide sequence of SEQ ID NO: 17; the immunoglobulin HC variable domain sequence is encoded by a nucleic acid comprising the nucleotide sequence of SEQ ID NO: 27, and the immunoglobulin LC variable domain sequence is encoded by a nucleic acid comprising the nucleotide sequence of SEQ ID NO: 37; the immunoglobulin HC variable domain sequence is encoded by a nucleic acid comprising the nucleotide sequence of SEQ ID NO: 47, and the immunoglobulin LC variable domain sequence is encoded by a nucleic acid comprising the nucleotide sequence of SEQ ID NO: 57; or the immunoglobulin HC variable domain sequence is encoded by a nucleic acid comprising the nucleotide sequence of SEQ ID NO: 67, and the immunoglobulin LC variable domain sequence is encoded by a nucleic acid comprising the nucleotide sequence of SEQ ID NO: 77. In another aspect, the isolated antibody or antigen-binding fragment thereof comprises: a heavy chain comprising an amino acid sequence having at least 95-99% identity to SEQ ID NO: 6, and a light chain comprising an amino acid sequence having at least 95-99% identity to SEQ ID NO: 16; a heavy chain comprising an amino acid sequence having at least 95-99% identity to SEQ ID NO: 26, and a light chain comprising an amino acid sequence having at least 95-99% identity to SEQ ID NO: 36; a heavy chain comprising an amino acid sequence having at least 95-99% identity to SEQ ID NO: 46, and a light chain comprising an amino acid sequence having at least 95-99% identity to SEQ ID NO: 56; or a heavy chain comprising an amino acid sequence having at least 95-99% identity to SEQ ID NO: 66, and a light chain comprising an amino acid sequence having at least 95-99% identity to SEQ ID NO: 76. In another aspect, the isolated antibody or antigen-binding fragment thereof comprises: a heavy chain comprising the amino acid sequence of SEQ ID NO: 6, and a light chain comprising the amino acid sequence of SEQ ID NO: 16; a heavy chain comprising the amino acid sequence of SEQ ID NO: 26, and a light chain comprising the amino acid sequence of SEQ ID NO: 36; a heavy chain comprising the amino acid sequence of SEQ ID NO: 46, and a light chain comprising the amino acid sequence of SEQ ID NO: 56; or a heavy chain comprising the amino acid sequence of SEQ ID NO: 66, and a light chain comprising the amino acid sequence of SEQ ID NO: 76. In another aspect, the isolated antibody or antigen-binding fragment thereof comprises: a heavy chain encoded by a nucleic acid comprising the nucleotide sequence of SEQ ID NO: 5, and a light chain encoded by a nucleic acid comprising the nucleotide sequence of SEQ ID NO: 15; a heavy chain encoded by a nucleic acid comprising the nucleotide sequence of SEQ ID NO: 25, and a light chain encoded by a nucleic acid comprising the nucleotide sequence of SEQ ID NO: 35; a heavy chain encoded by a nucleic acid comprising the nucleotide sequence of SEQ ID NO: 45, and a light chain encoded by a nucleic acid comprising the nucleotide sequence of SEQ ID NO: 55; or a heavy chain encoded by a nucleic acid comprising the nucleotide sequence of SEQ ID NO: 65, and a light chain encoded by a nucleic acid comprising the nucleotide sequence of SEQ ID NO: 75. In another aspect, the isolated antibody or antigen-binding fragment thereof immunospecifically binds to an epitope of a TDP-43 protein or fragment thereof. In another aspect, the isolated antibody or antigen-binding fragment thereof immunospecifically binds to at least a portion of the TDP-43 protein of SEQ ID NO: 2. In another aspect, the isolated antibody or antigen-binding fragment thereof immunospecifically binds to a pathologic TDP-43 protein or fragment thereof.

Another embodiment described herein is an isolated nucleic acid comprising a nucleotide sequence encoding the antibody or antigen-binding fragment thereof of described herein.

Another embodiment described herein is a vector comprising an isolated nucleic acid described herein.

Another embodiment described herein is a cell comprising the vector described herein. Another embodiment described herein is an immunoassay comprising one or more of the antibodies or antigen-binding fragments thereof described herein.

Another embodiment described herein is the use of one or more of the antibodies or antigen-binding fragments thereof described herein to prescreen a subject for a clinical trial.

Another embodiment described herein is an immunoassay method for detecting TAR DNA-binding protein 43 (TDP-43) protein, the method comprising: contacting a biological sample suspected of having a TDP-43 protein or fragment thereof with one or more antibodies or antigen-binding fragments thereof capable of binding to a TDP-43 protein or fragment thereof, the antibodies or antigen-binding fragments thereof comprising an immunoglobulin heavy chain (HC) variable domain sequence and an immunoglobulin light chain (LC) variable domain sequence, wherein: the immunoglobulin HC variable domain sequence comprises: (i) an HC CDR1 comprising an amino acid sequence having at least 95-99% identity to SEQ ID NO: 10, (ii) an HC CDR2 comprising an amino acid sequence having at least 95-99% identity to SEQ ID NO: 12, and (iii) an HC CDR3 comprising an amino acid sequence having at least 95-99% identity to SEQ ID NO: 14; and the immunoglobulin LC variable domain sequence comprises: (i) an LC CDR1 comprising an amino acid sequence having at least 95-99% identity to SEQ ID NO: 20, (ii) an LC CDR2 comprising an amino acid sequence having at least 95-99% identity to SEQ ID NO: 22, and (iii) an LC CDR3 comprising an amino acid sequence having at least 95-99% identity to SEQ ID NO: 24; the immunoglobulin HC variable domain sequence comprises: (i) an HC CDR1 comprising an amino acid sequence having at least 95-99% identity to SEQ ID NO: 30, (ii) an HC CDR2 comprising an amino acid sequence having at least 95-99% identity to SEQ ID NO: 32, and (iii) an HC CDR3 comprising an amino acid sequence having at least 95-99% identity to SEQ ID NO: 34; and the immunoglobulin LC variable domain sequence comprises: (i) an LC CDR1 comprising an amino acid sequence having at least 95-99% identity to SEQ ID NO: 40, (ii) an LC CDR2 comprising an amino acid sequence having at least 95-99% identity to SEQ ID NO: 42, and (iii) an LC CDR3 comprising an amino acid sequence having at least 95-99% identity to SEQ ID NO: 44; the immunoglobulin HC variable domain sequence comprises: (i) an HC CDR1 comprising an amino acid sequence having at least 95-99% identity to SEQ ID NO: 50, (ii) an HC CDR2 comprising an amino acid sequence having at least 95-99% identity to SEQ ID NO: 52, and (iii) an HC CDR3 comprising an amino acid sequence having at least 95-99% identity to SEQ ID NO: 54; and the immunoglobulin LC variable domain sequence comprises: (i) an LC CDR1 comprising an amino acid sequence having at least 95-99% identity to SEQ ID NO: 60, (ii) an LC CDR2 comprising an amino acid sequence having at least 95-99% identity to SEQ ID NO: 62, and (iii) an LC CDR3 comprising an amino acid sequence having at least 95-99% identity to SEQ ID NO: 64; or the immunoglobulin HC variable domain sequence comprises: (i) an HC CDR1 comprising an amino acid sequence having at least 95-99% identity to SEQ ID NO: 70, (ii) an HC CDR2 comprising an amino acid sequence having at least 95-99% identity to SEQ ID NO: 72, and (iii) an HC CDR3 comprising an amino acid sequence having at least 95-99% identity to SEQ ID NO: 74; and the immunoglobulin LC variable domain sequence comprises: (i) an LC CDR1 comprising an amino acid sequence having at least 95-99% identity to SEQ ID NO: 80, (ii) an LC CDR2 comprising an amino acid sequence having at least 95-99% identity to SEQ ID NO: 82, and (iii) an LC CDR3 comprising an amino acid sequence having at least 95-99% identity to SEQ ID NO: 84; and detecting a presence or absence of TDP-43 protein in the sample based on a measured binding or lack of binding of the one or more antibodies or antigen-binding fragments thereof to the sample. In one aspect, the method further comprises treating the sample with one or more solubilizing reagents. In another aspect, the one or more solubilizing reagents comprises 1-3% sodium dodecyl sulfate (SDS) detergent. In another aspect, the method further comprises subjecting the sample to one or more freeze-thaw cycles. In another aspect, detecting comprises immunohistochemistry (IHC), Meso Scale Discovery (MSD) biomarker assay, Western blotting, flow cytometry, radioimmunoassay (RIA), counting immunoassay (CIA), enzyme immunoassays (EIA) or enzyme-linked immunosorbent assays (ELISA), sandwich ELISA, fluoroimmunoassay (FIA), chemiluminescence immunoassay (CLIA), or combinations thereof. In another aspect, the biological sample comprises whole blood, serum, plasma, or cerebrospinal fluid (CSF). In another aspect, the biological sample comprises a cell or a tissue sample. In another aspect, the biological sample comprises extracellular vesicles (EVs), and wherein at least a portion of detected TDP-43 protein is derived from EVs. In another aspect, the biological sample is obtained from a subject that is diagnosed as having, suspected as having, or at risk of having or developing a neurodegenerative disease. In another aspect, the neurodegenerative disease is frontotemporal lobar degeneration (FTLD). In another aspect, the TDP-43 protein is misfolded. In another aspect, the TDP-43 protein is a pathologic TDP-43 protein. In another aspect, one or more of the antibodies or antigen-binding fragments thereof are biotinylated. In another aspect, the method further comprises contacting the sample with the detection peptide of SEQ ID NO: 85.

Another embodiment described herein is a method of detecting pathologic TAR DNA-binding protein 43 (TDP-43) protein in a subject, the method comprising: contacting a biological sample from the subject with one or more antibodies or antigen-binding fragments thereof capable of binding to a TDP-43 protein or fragment thereof, the antibodies or antigen-binding fragments thereof comprising an immunoglobulin heavy chain (HC) variable domain sequence and an immunoglobulin light chain (LC) variable domain sequence, wherein: the immunoglobulin HC variable domain sequence comprises: (i) an HC CDR1 comprising an amino acid sequence having at least 95-99% identity to SEQ ID NO: 10, (ii) an HC CDR2 comprising an amino acid sequence having at least 95-99% identity to SEQ ID NO: 12, and (iii) an HC CDR3 comprising an amino acid sequence having at least 95-99% identity to SEQ ID NO: 14; and the immunoglobulin LC variable domain sequence comprises: (i) an LC CDR1 comprising an amino acid sequence having at least 95-99% identity to SEQ ID NO: 20, (ii) an LC CDR2 comprising an amino acid sequence having at least 95-99% identity to SEQ ID NO: 22, and (iii) an LC CDR3 comprising an amino acid sequence having at least 95-99% identity to SEQ ID NO: 24; the immunoglobulin HC variable domain sequence comprises: (i) an HC CDR1 comprising an amino acid sequence having at least 95-99% identity to SEQ ID NO: 30, (ii) an HC CDR2 comprising an amino acid sequence having at least 95-99% identity to SEQ ID NO: 32, and (iii) an HC CDR3 comprising an amino acid sequence having at least 95-99% identity to SEQ ID NO: 34; and the immunoglobulin LC variable domain sequence comprises: (i) an LC CDR 1 comprising an amino acid sequence having at least 95-99% identity to SEQ ID NO: 40, (ii) an LC CDR2 comprising an amino acid sequence having at least 95-99% identity to SEQ ID NO: 42, and (iii) an LC CDR3 comprising an amino acid sequence having at least 95-99% identity to SEQ ID NO: 44; the immunoglobulin HC variable domain sequence comprises: (i) an HC CDR1 comprising an amino acid sequence having at least 95-99% identity to SEQ ID NO: 50, (ii) an HC CDR2 comprising an amino acid sequence having at least 95-99% identity to SEQ ID NO: 52, and (iii) an HC CDR3 comprising an amino acid sequence having at least 95-99% identity to SEQ ID NO: 54; and the immunoglobulin LC variable domain sequence comprises: (i) an LC CDR1 comprising an amino acid sequence having at least 95-99% identity to SEQ ID NO: 60, (ii) an LC CDR2 comprising an amino acid sequence having at least 95-99% identity to SEQ ID NO: 62, and (iii) an LC CDR3 comprising an amino acid sequence having at least 95-99% identity to SEQ ID NO: 64; or the immunoglobulin HC variable domain sequence comprises: (i) an HC CDR 1 comprising an amino acid sequence having at least 95-99% identity to SEQ ID NO: 70, (ii) an HC CDR2 comprising an amino acid sequence having at least 95-99% identity to SEQ ID NO: 72, and (iii) an HC CDR3 comprising an amino acid sequence having at least 95-99% identity to SEQ ID NO: 74; and the immunoglobulin LC variable domain sequence comprises: (i) an LC CDR1 comprising an amino acid sequence having at least 95-99% identity to SEQ ID NO: 80, (ii) an LC CDR2 comprising an amino acid sequence having at least 95-99% identity to SEQ ID NO: 82, and (iii) an LC CDR3 comprising an amino acid sequence having at least 95-99% identity to SEQ ID NO: 84; and detecting a presence or absence of pathologic TDP-43 protein in the subject based on a measured binding or lack of binding of the one or more antibodies or antigen-binding fragments thereof to the sample. In one aspect, the pathologic TDP-43 protein is misfolded. In another aspect, the pathologic TDP-43 protein is phosphorylated at one or more residues. In another aspect, the subject has a neurodegenerative disease comprising frontotemporal lobar degeneration (FTLD).

Another embodiment described herein is a method of diagnosing and treating a subject having a neurodegenerative disease, the method comprising: contacting a sample from the subject with one or more antibodies or antigen-binding fragments thereof capable of binding to a TAR DNA-binding protein 43 (TDP-43) protein or fragment thereof, the antibodies or antigen-binding fragments thereof comprising an immunoglobulin heavy chain (HC) variable domain sequence and an immunoglobulin light chain (LC) variable domain sequence, wherein: the immunoglobulin HC variable domain sequence comprises: (i) an HC CDR1 comprising an amino acid sequence having at least 95-99% identity to SEQ ID NO: 10, (ii) an HC CDR2 comprising an amino acid sequence having at least 95-99% identity to SEQ ID NO: 12, and (iii) an HC CDR3 comprising an amino acid sequence having at least 95-99% identity to SEQ ID NO: 14; and the immunoglobulin LC variable domain sequence comprises: (i) an LC CDR1 comprising an amino acid sequence having at least 95-99% identity to SEQ ID NO: 20, (ii) an LC CDR2 comprising an amino acid sequence having at least 95-99% identity to SEQ ID NO: 22, and (iii) an LC CDR3 comprising an amino acid sequence having at least 95-99% identity to SEQ ID NO: 24; the immunoglobulin HC variable domain sequence comprises: (i) an HC CDR1 comprising an amino acid sequence having at least 95-99% identity to SEQ ID NO: 30, (ii) an HC CDR2 comprising an amino acid sequence having at least 95-99% identity to SEQ ID NO: 32, and (iii) an HC CDR3 comprising an amino acid sequence having at least 95-99% identity to SEQ ID NO: 34; and the immunoglobulin LC variable domain sequence comprises: (i) an LC CDR1 comprising an amino acid sequence having at least 95-99% identity to SEQ ID NO: 40, (ii) an LC CDR2 comprising an amino acid sequence having at least 95-99% identity to SEQ ID NO: 42, and (iii) an LC CDR3 comprising an amino acid sequence having at least 95-99% identity to SEQ ID NO: 44; the immunoglobulin HC variable domain sequence comprises: (i) an HC CDR1 comprising an amino acid sequence having at least 95-99% identity to SEQ ID NO: 50, (ii) an HC CDR2 comprising an amino acid sequence having at least 95-99% identity to SEQ ID NO: 52, and (iii) an HC CDR3 comprising an amino acid sequence having at least 95-99% identity to SEQ ID NO: 54; and the immunoglobulin LC variable domain sequence comprises: (i) an LC CDR1 comprising an amino acid sequence having at least 95-99% identity to SEQ ID NO: 60, (ii) an LC CDR2 comprising an amino acid sequence having at least 95-99% identity to SEQ ID NO: 62, and (iii) an LC CDR3 comprising an amino acid sequence having at least 95-99% identity to SEQ ID NO: 64; or the immunoglobulin HC variable domain sequence comprises: (i) an HC CDR1 comprising an amino acid sequence having at least 95-99% identity to SEQ ID NO: 70, (ii) an HC CDR2 comprising an amino acid sequence having at least 95-99% identity to SEQ ID NO: 72, and (iii) an HC CDR3 comprising an amino acid sequence having at least 95-99% identity to SEQ ID NO: 74; and the immunoglobulin LC variable domain sequence comprises: (i) an LC CDR1 comprising an amino acid sequence having at least 95-99% identity to SEQ ID NO: 80, (ii) an LC CDR2 comprising an amino acid sequence having at least 95-99% identity to SEQ ID NO: 82, and (iii) an LC CDR3 comprising an amino acid sequence having at least 95-99% identity to SEQ ID NO: 84; detecting a presence or absence of binding of the one or more antibodies or antigen-binding fragments thereof to the sample; diagnosing the subject with a neurodegenerative disease when antibody binding is detected; and treating the subject with one or more anti-neurodegenerative disease therapies. In one aspect, the sample comprises whole blood, serum, plasma, or cerebrospinal fluid (CSF). In another aspect, the neurodegenerative disease is frontotemporal lobar degeneration (FTLD). In another aspect, the one or more anti-neurodegenerative disease therapies comprises selective serotonin reuptake inhibitors (SSRIs), antipsychotics, speech and language therapy, physical and occupational therapy, Chimeric Antigen Receptor T-cell (CAR-T) therapy targeting neurons or microglia, Proteolysis-Targeting Chimera (PROTAC) therapy, or combinations thereof. In another aspect, the subject is a human.

Another embodiment described herein is a method for selecting whether to enroll a subject in a clinical trial for frontotemporal lobar degeneration with TAR DNA-binding protein 43 inclusions (FTLD-TDP), the method comprising: measuring protein expression levels of TAR DNA-binding protein 43 (TDP-43) in a sample from a subject, wherein the measuring comprises contacting the sample with one or more antibodies or antigen-binding fragments thereof capable of binding to a TDP-43 protein or fragment thereof, the antibodies or antigen-binding fragments thereof comprising an immunoglobulin heavy chain (HC) variable domain sequence and an immunoglobulin light chain (LC) variable domain sequence, wherein: the immunoglobulin HC variable domain sequence comprises: (i) an HC CDR1 comprising an amino acid sequence having at least 95-99% identity to SEQ ID NO: 10, (ii) an HC CDR2 comprising an amino acid sequence having at least 95-99% identity to SEQ ID NO: 12, and (iii) an HC CDR3 comprising an amino acid sequence having at least 95-99% identity to SEQ ID NO: 14; and the immunoglobulin LC variable domain sequence comprises: (i) an LC CDR1 comprising an amino acid sequence having at least 95-99% identity to SEQ ID NO: 20, (ii) an LC CDR2 comprising an amino acid sequence having at least 95-99% identity to SEQ ID NO: 22, and (iii) an LC CDR3 comprising an amino acid sequence having at least 95-99% identity to SEQ ID NO: 24; the immunoglobulin HC variable domain sequence comprises: (i) an HC CDR1 comprising an amino acid sequence having at least 95-99% identity to SEQ ID NO: 30, (ii) an HC CDR2 comprising an amino acid sequence having at least 95-99% identity to SEQ ID NO: 32, and (iii) an HC CDR3 comprising an amino acid sequence having at least 95-99% identity to SEQ ID NO: 34; and the immunoglobulin LC variable domain sequence comprises: (i) an LC CDR1 comprising an amino acid sequence having at least 95-99% identity to SEQ ID NO: 40, (ii) an LC CDR2 comprising an amino acid sequence having at least 95-99% identity to SEQ ID NO: 42, and (iii) an LC CDR3 comprising an amino acid sequence having at least 95-99% identity to SEQ ID NO: 44; the immunoglobulin HC variable domain sequence comprises: (i) an HC CDR1 comprising an amino acid sequence having at least 95-99% identity to SEQ ID NO: 50, (ii) an HC CDR2 comprising an amino acid sequence having at least 95-99% identity to SEQ ID NO: 52, and (iii) an HC CDR3 comprising an amino acid sequence having at least 95-99% identity to SEQ ID NO: 54; and the immunoglobulin LC variable domain sequence comprises: (i) an LC CDR1 comprising an amino acid sequence having at least 95-99% identity to SEQ ID NO: 60, (ii) an LC CDR2 comprising an amino acid sequence having at least 95-99% identity to SEQ ID NO: 62, and (iii) an LC CDR3 comprising an amino acid sequence having at least 95-99% identity to SEQ ID NO: 64; or the immunoglobulin HC variable domain sequence comprises: (i) an HC CDR1 comprising an amino acid sequence having at least 95-99% identity to SEQ ID NO: 70, (ii) an HC CDR2 comprising an amino acid sequence having at least 95-99% identity to SEQ ID NO: 72, and (iii) an HC CDR3 comprising an amino acid sequence having at least 95-99% identity to SEQ ID NO: 74; and the immunoglobulin LC variable domain sequence comprises: (i) an LC CDR1 comprising an amino acid sequence having at least 95-99% identity to SEQ ID NO: 80, (ii) an LC CDR2 comprising an amino acid sequence having at least 95-99% identity to SEQ ID NO: 82, and (iii) an LC CDR3 comprising an amino acid sequence having at least 95-99% identity to SEQ ID NO: 84; comparing measured protein expression levels of TDP-43 to a threshold TDP-43 expression level; and when the measured protein expression levels of TDP-43 are greater than the threshold TDP-43 expression level, selecting the subject for the clinical trial; or when the measured protein expression levels of TDP-43 are less than the threshold TDP-43 expression level, not selecting the subject for the clinical trial.

Another embodiment described herein is a method for isolating and detecting a protein biomarker in a biological sample comprising extracellular vesicles (EVs), the method comprising: treating the sample with a solubilizing reagent comprising about 1-3% sodium dodecyl sulfate (SDS) detergent to lyse the EVs; and detecting a presence or absence of the protein biomarker based on a measured binding or lack of binding of the sample to one or more antibodies or antigen-binding fragments thereof that are specific to the protein biomarker. In one aspect, the sample is treated with the solubilizing reagent at room temperature for a period of time of about 5 minutes to about 15 minutes. In another aspect, the method further comprises centrifuging the sample. In another aspect, the method further comprises subjecting the sample to one or more freeze-thaw cycles. In another aspect, the biological sample comprises whole blood, serum, plasma, or cerebrospinal fluid (CSF). In another aspect, the biological sample is obtained from a subject that is diagnosed as having, suspected as having, or at risk of having or developing a neurodegenerative disease. In another aspect, detecting comprises immunohistochemistry (IHC), Meso Scale Discovery (MSD) biomarker assay, Western blotting, flow cytometry, radioimmunoassay (RIA), counting immunoassay (CIA), enzyme immunoassays (EIA) or enzyme-linked immunosorbent assays (ELISA), sandwich ELISA, fluoroimmunoassay (FIA), chemiluminescence immunoassay (CLIA), or combinations thereof. In another aspect, the protein biomarker is a TAR DNA-binding protein 43 (TDP-43) protein or fragment thereof. In another aspect, detecting comprises contacting the sample with one or more antibodies or antigen-binding fragments thereof capable of binding to a TDP-43 protein or fragment thereof, the antibodies or antigen-binding fragments thereof comprising an immunoglobulin heavy chain (HC) variable domain sequence and an immunoglobulin light chain (LC) variable domain sequence, wherein: the immunoglobulin HC variable domain sequence comprises: (i) an HC CDR1 comprising an amino acid sequence having at least 95-99% identity to SEQ ID NO: 10, (ii) an HC CDR2 comprising an amino acid sequence having at least 95-99% identity to SEQ ID NO: 12, and (iii) an HC CDR3 comprising an amino acid sequence having at least 95-99% identity to SEQ ID NO: 14; and the immunoglobulin LC variable domain sequence comprises: (i) an LC CDR1 comprising an amino acid sequence having at least 95-99% identity to SEQ ID NO: 20, (ii) an LC CDR2 comprising an amino acid sequence having at least 95-99% identity to SEQ ID NO: 22, and (iii) an LC CDR3 comprising an amino acid sequence having at least 95-99% identity to SEQ ID NO: 24; the immunoglobulin HC variable domain sequence comprises: (i) an HC CDR1 comprising an amino acid sequence having at least 95-99% identity to SEQ ID NO: 30, (ii) an HC CDR2 comprising an amino acid sequence having at least 95-99% identity to SEQ ID NO: 32, and (iii) an HC CDR3 comprising an amino acid sequence having at least 95-99% identity to SEQ ID NO: 34; and the immunoglobulin LC variable domain sequence comprises: (i) an LC CDR1 comprising an amino acid sequence having at least 95-99% identity to SEQ ID NO: 40, (ii) an LC CDR2 comprising an amino acid sequence having at least 95-99% identity to SEQ ID NO: 42, and (iii) an LC CDR3 comprising an amino acid sequence having at least 95-99% identity to SEQ ID NO: 44; the immunoglobulin HC variable domain sequence comprises: (i) an HC CDR1 comprising an amino acid sequence having at least 95-99% identity to SEQ ID NO: 50, (ii) an HC CDR2 comprising an amino acid sequence having at least 95-99% identity to SEQ ID NO: 52, and (iii) an HC CDR3 comprising an amino acid sequence having at least 95-99% identity to SEQ ID NO: 54; and the immunoglobulin LC variable domain sequence comprises: (i) an LC CDR1 comprising an amino acid sequence having at least 95-99% identity to SEQ ID NO: 60, (ii) an LC CDR2 comprising an amino acid sequence having at least 95-99% identity to SEQ ID NO: 62, and (iii) an LC CDR3 comprising an amino acid sequence having at least 95-99% identity to SEQ ID NO: 64; or the immunoglobulin HC variable domain sequence comprises: (i) an HC CDR1 comprising an amino acid sequence having at least 95-99% identity to SEQ ID NO: 70, (ii) an HC CDR2 comprising an amino acid sequence having at least 95-99% identity to SEQ ID NO: 72, and (iii) an HC CDR3 comprising an amino acid sequence having at least 95-99% identity to SEQ ID NO: 74; and the immunoglobulin LC variable domain sequence comprises: (i) an LC CDR1 comprising an amino acid sequence having at least 95-99% identity to SEQ ID NO: 80, (ii) an LC CDR2 comprising an amino acid sequence having at least 95-99% identity to SEQ ID NO: 82, and (iii) an LC CDR3 comprising an amino acid sequence having at least 95-99% identity to SEQ ID NO: 84.

Another embodiment described herein is an immunoassay kit for selectively detecting a TAR DNA-binding protein 43 (TDP-43) protein or fragment thereof in a biological sample, the kit comprising: one or more antibodies or antigen-binding fragments thereof capable of binding to a TDP-43 protein or fragment thereof, the antibodies or antigen-binding fragments thereof comprising an immunoglobulin heavy chain (HC) variable domain sequence and an immunoglobulin light chain (LC) variable domain sequence, wherein: the immunoglobulin HC variable domain sequence comprises: (i) an HC CDR1 comprising an amino acid sequence having at least 95-99% identity to SEQ ID NO: 10, (ii) an HC CDR2 comprising an amino acid sequence having at least 95-99% identity to SEQ ID NO: 12, and (iii) an HC CDR3 comprising an amino acid sequence having at least 95-99% identity to SEQ ID NO: 14; and the immunoglobulin LC variable domain sequence comprises: (i) an LC CDR1 comprising an amino acid sequence having at least 95-99% identity to SEQ ID NO: 20, (ii) an LC CDR2 comprising an amino acid sequence having at least 95-99% identity to SEQ ID NO: 22, and (iii) an LC CDR3 comprising an amino acid sequence having at least 95-99% identity to SEQ ID NO: 24; the immunoglobulin HC variable domain sequence comprises: (i) an HC CDR1 comprising an amino acid sequence having at least 95-99% identity to SEQ ID NO: 30, (ii) an HC CDR2 comprising an amino acid sequence having at least 95-99% identity to SEQ ID NO: 32, and (iii) an HC CDR3 comprising an amino acid sequence having at least 95-99% identity to SEQ ID NO: 34; and the immunoglobulin LC variable domain sequence comprises: (i) an LC CDR1 comprising an amino acid sequence having at least 95-99% identity to SEQ ID NO: 40, (ii) an LC CDR2 comprising an amino acid sequence having at least 95-99% identity to SEQ ID NO: 42, and (iii) an LC CDR3 comprising an amino acid sequence having at least 95-99% identity to SEQ ID NO: 44; the immunoglobulin HC variable domain sequence comprises: (i) an HC CDR1 comprising an amino acid sequence having at least 95-99% identity to SEQ ID NO: 50, (ii) an HC CDR2 comprising an amino acid sequence having at least 95-99% identity to SEQ ID NO: 52, and (iii) an HC CDR3 comprising an amino acid sequence having at least 95-99% identity to SEQ ID NO: 54; and the immunoglobulin LC variable domain sequence comprises: (i) an LC CDR1 comprising an amino acid sequence having at least 95-99% identity to SEQ ID NO: 60, (ii) an LC CDR2 comprising an amino acid sequence having at least 95-99% identity to SEQ ID NO: 62, and (iii) an LC CDR3 comprising an amino acid sequence having at least 95-99% identity to SEQ ID NO: 64; or the immunoglobulin HC variable domain sequence comprises: (i) an HC CDR 1 comprising an amino acid sequence having at least 95-99% identity to SEQ ID NO: 70, (ii) an HC CDR2 comprising an amino acid sequence having at least 95-99% identity to SEQ ID NO: 72, and (iii) an HC CDR3 comprising an amino acid sequence having at least 95-99% identity to SEQ ID NO: 74; and the immunoglobulin LC variable domain sequence comprises: (i) an LC CDR1 comprising an amino acid sequence having at least 95-99% identity to SEQ ID NO: 80, (ii) an LC CDR2 comprising an amino acid sequence having at least 95-99% identity to SEQ ID NO: 82, and (iii) an LC CDR3 comprising an amino acid sequence having at least 95-99% identity to SEQ ID NO: 84; a detection reagent; optionally, buffers and receptacles; and optionally, one or more of packaging or instruction for use. In one aspect, the kit further comprises a solid support for the one or more antibodies or antigen-binding fragments thereof. In one aspect, the kit further comprises a detection means. In one aspect, the detection means is one or more of fluorescent, luminescent, radioactive, or colorimetric. In one aspect, the detection reagent comprises colorimetric substrates, chemiluminescent substrates, fluorescent substrates, or combinations thereof.

Another embodiment described herein is the use of one or more antibodies or antigen-binding fragments thereof described herein in an immunoassay for detecting TAR DNA-binding protein 43 (TDP-43) protein in a biological sample.

Another embodiment described herein is the use of one or more antibodies or antigen-binding fragments thereof described herein in a method of determining a subject as having a neurodegenerative disease.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows a schematic showing epitopes in TDP-43 recognized by monoclonal antibodies (MAbs) Nos. 3, 4, 9, and 6.

FIG. 2 shows immunostaining images showing that MAb No. 9 preferentially recognizes pathologic TDP-43. MAb No. 9 robustly detects pathologic TDP-43 inclusions shown by the arrows with no or very minimal nuclear staining in the frontal cortex of an individual who suffered from FTLD. This was similar to a known antibody (aa 409/410) that is specific for phospho TDP-43. In contrast, MAb No. 6 recognizes both pathologic TDP-43 inclusions (arrows) and normal nuclear TDP-43 (asterisk) in the frontal cortex of an individual who suffered from FTLD. The scale bar is 20 μm.

FIG. 3 shows images of immunoblotting of CSF samples from subjects with FTLD-TDP and FTLD-tau using MAb No. 9 and 409/410 antibodies. MAb No. 9 revealed strong 20 kDa and 30-35 kDa fragments (shown by the asterisk) in FTLD-TDP subjects which are not seen when the 409/410 antibody was used.

FIG. 4 shows images of immunoprecipitation of pathologic TDP-43 from CSF of a subject who suffered from FTLD-TDP using MAb No. 9.

FIG. 5A-5B show immunofluorescent and immunostaining images to analyze the specificity of MAb No. 9. FIG. 5A is images of immunofluorescent staining with MAb No. 9. The immunofluorescent staining images reveal immunopositivity in HEK293 cells transfected with a TDP-43-expressing plasmid (arrows). The p409/410 MAb was a positive control and the mouse IgG was a negative control. FIG. 5B shows images of MAb No. 9 immunostaining. Immunoreactivity of MAb No. 9 was ablated by competition with a peptide harboring the epitope recognized by MAb No. 9 in anterior horn motor neurons (arrows) of a subject who suffered from ALS. The scale bar is 50 μm.

FIG. 6A-6B show that MAb No. 9 detects pathologic TDP-43 in plasma. FIG. 6A is a bar chart showing absorbance values from an ELISA using MAb No. 9 against plasma samples from subjects diagnosed with various pathologies. FIG. 6B is images of immunoblotting of the plasma samples with the p409/410 MAb and MAb No. 9. Type A1 is FTLD-TDP Type A sample 1; AD+TDP is AD with TDP-43 pathology; AD-TDP is AD without TDP-43 pathology; PSP is progressive supranuclear palsy; CBD is corticobasal degeneration.

FIG. 7A-7C show that MAb No. 9 detects a novel TDP-43 pathology. FIG. 7A is images of immunostaining with MAb No. 9 and p409/410 MAb. MAb No. 9 detects similar pathologic TDP-43 inclusions as those seen with the p409/410 MAb (arrows), except for more fine neurites in the frontal cortex of a subject who suffered from FTLD-TDP Type A (arrowhead), novel fine neurites in the frontal cortex of Type B (arrowhead), and novel inclusions in the hippocampus of a subject who suffered from AD with TDP pathology (arrowhead). In Type A, the density of the MAb No. 9-positive inclusions is highest in layer 2 (inset of hematoxylin and eosin [H&E] image), which has the most severe neuronal loss of all layers. The scale bar is 50 μm. FIG. 7B is immunofluorescent images of MAb No. 9 immunostaining that reveals more TDP-43 pathology (arrowheads) in the anterior horn motor neurons of a subject who suffered from ALS with a C9 mutation. The arrow shows the only inclusion positive for both the p409/410 MAb and MAb No. 9. The scale bar is 50 μm. FIG. 7C is a bar chart showing semiquantification of TDP-43 pathology. **, p<0.01; n=5.

FIG. 8 shows images of immunofluorescent staining showing MAb No. 9-immunopositivity (cy3, orange) is co-localized (arrows) with beta-amyloid (FITC, green), which has not been reported before, and tau (FITC, green) in the amygdala of a subject who suffered from AD with TDP pathology brain. The scale bar is 50 μm.

FIG. 9 shows a scatter dot plot showing TDP-43 levels in plasma from subjects who suffered from FTLD-TDP (n=35), FTLD-Tau (n=34), and AD (n=22). The comparisons between TDP-43 concentration distribution were calculated by the Mann-Whitney U test. The median of plasma TDP-43 concentrations were 5.8, 5.4, and 4.0 ng/ml in AD, FTLD-TDP, and FTLD-Tau subjects.

FIG. 10A-J show MAb No. 9 immunoreactivity in FTLD-TDP Type A. FIG. 10A shows that p409/410 MAb revealed small, compact NCI and short DN in the frontal cortex. FIG. 10B-G show MAb No. 9 robustly detected pathological TDP-43 inclusions, with weak nuclear staining, but MAb No. 9 exhibited a higher extent of TDP-43 pathology overall. FIG. 10B shows in the cortical and subcortical regions of FTLD-TDP Type A cases, MAb No. 9 immunohistochemistry labeled all characteristic inclusion types, such as NCI, NII (inset), DN, and ThD in the frontal cortex; FIG. 10C shows NCI in the dentate gyrus. FIG. 10D shows DN and ThD in the CA1 region of the hippocampus. FIG. 10E shows NCI, DN, and ThD in striatum. FIG. 10F shows NCI in the substantia nigra. FIG. 10G shows thread-like pathology in the white matter. Scale bar, 50 μm. FIG. 10H-J show double-label immunofluorescence revealing that MAb No. 9 exhibits a higher density of immunoreactivity compared to p409/410 PAb in FTLD-TDP Type A cases. Colocalization of MAb No. 9 (Cy3, orange-red) and p409/410 PAb (green) is confirmed by merged images showing yellow signals (arrow), with a Pearson's correlation coefficient of 0.588. Notably, MAb No. 9 also detects additional novel TDP-43-positive structures, visualized as fine red dot-like signals (arrowheads) in the frontal cortex (FIG. 10A), dentate gyrus (FIG. 10B), and hippocampal CA1 region (FIG. 10C). Nuclei are counterstained with DAPI (blue) in the merged images. Scale bar: 50 μm.

FIG. 11A-J show MAb No. 9 immunoreactivity in FTLD-TDP Type B. The p409/410 MAb revealed small, compact NCI, short DN, and ThD in the frontal cortex (FIG. 11A), while MAb No. 9 exhibited a higher extent of TDP-43 pathology overall (FIG. 11B-G). FIG. 11B shows in cortical and subcortical regions of FTLD-TDP Type B cases, MAb No. 9 immunohistochemistry labeled all characteristic types of inclusions, such as NCI, DN, and ThD pathology in the frontal cortex. FIG. 11C shows NCI in the dentate gyrus. FIG. 11D-E show NCI, DN, and ThD pathology in the motor cortex and striatum. FIG. 11F shows NCI in the lower motor neuron. FIG. 11G shows GCI pathology in the white matter. Scale bar, 50 μm. Double-label immunofluorescence demonstrates that MAb No. 9 exhibits a higher density of immunoreactivity compared to p409/410 PAb in FTLD-TDP Type B (FIG. 11H-J). Colocalization of MAb No. 9 (Cy3, orange-red) and p409/410 PAb (green) is confirmed in merged images by yellow signals (arrows), with a Pearson's correlation coefficient of 0.621. Importantly, MAb No. 9 also reveals additional novel TDP-43-positive structures, visualized as fine red dot-like signals (arrowheads) in the frontal cortex (FIG. 11A), dentate gyrus (FIG. 11B), and lower motor neurons (FIG. 11C). Nuclei are counterstained with DAPI (blue) in the merged images. Scale bar: 50 μm.

FIG. 12A-E show MAb No. 9 immunoreactivity in ALS-TDP. MAb No. 9 immunohistochemistry robustly labels the complete spectrum of pathological TDP-43 inclusions present in ALS-TDP. FIG. 12A shows specifically, diffuse and compact NCI in the spinal cord. FIG. 12B shows predominantly diffuse NCI and abundant dot/thread like neuropil staining in the precentral gyrus. FIG. 12C shows oligodendroglial GCI in the white matter. Scale bar, 50 μm. Double-label immunofluorescence for MAb No. 9 (Cy3, orange-red) and p409/410 PAb (green) confirmed colocalization (yellow in merged image, arrows) (Pearson's coefficient value of 0.621), yet revealed additional TDP-43 positivity, indicated by fine red dot-like stains (arrowheads) in the merged image in the lower motor neurons (FIG. 12D), and dentate gyrus (FIG. 12E). Nuclei stained with Dapi (blue) in merged images. Scale bar, 50 μm.

FIG. 13A-I show MAb No. 9 immunopositivity remains prominent in advanced stages of FTLD-TDP Types A and B, as well as ALS-TDP. FIGS. 13A, D, and G show representative cortical regions from a FTLD-TDP Type A case showing varying degrees of neuronal loss mild (FIG. 13A); moderate (FIG. 13D); and severe (FIG. 13G). Corresponding immunohistochemical staining with p409/410 and MAb No. 9 is shown for each region. MAb No. 9 immunoreactivity remains abundant in areas with severe neuronal loss (FIG. 13H), in contrast to p409/410, which shows reduced staining in these regions (FIG. 13I). Scale bar, 200 μm in FIG. 13A, FIG. 13D, and FIG. 13G; 100 μm in FIG. 13B, FIG. 13C, FIG. 13E, FIG. 13F, FIG. 13H, and FIG. 13I.

FIG. 14A-G show MAb No. 9 immunoreactivity in FTLD-TDP Type C. MAb No. 9 immunohistochemistry labeled all characteristic types of inclusions in FTLD-TDP Type C cases, including long DN in the frontal cortex (FIG. 14A), compact Pick-like NCI in the dentate gyrus (FIG. 14B), and NCI and DN in the striatum (FIG. 14C). Double-label immunofluorescence showed robust co-labeling of p409/410 PAb (green) with MAb No. 9 (orange-red), as shown for the long DN in the frontal cortex (FIG. 14D) and NCI in the dentate gyrus (FIG. 14E). Nuclei stained with Dapi (blue) in merged images. Scale bar, 50 μm. Semiquantitative analysis of TDP-43 pathology in the frontal cortices of FTLD-TDP Type A, Type B, and Type C brains, and in the motor cortex of ALS-TDP brains. **, p<0.01; *, p<0.05 (p409/410 MAb vs. MAb No. 9). Sample size n=10 for Type A, Type B, and ALS; n=5 for Type C (FIG. 14F). Immunoblot analysis of TDP-43 in FTLD-TDP subtypes and control brain tissue using MAb No. 9 (FIG. 14G). Sarkosyl-insoluble protein fractions were extracted from frozen frontal cortical tissues of FTLD-TDP Type A, Type B, Type C, and neurologically normal control brains. MAb No. 9 detected a prominent 43-kDa band (arrow) corresponding to full-length TDP-43 in all FTLD-TDP subtypes, with only a faint physiological band observed in the control sample. The 43-kDa bands were abolished following absorption with a peptide containing the epitope recognized by MAb No. 9.

FIG. 15A-G show MAb No. 9 immunoreactivity in ADNC with LATE-NC stage 3. Similar to antibodies specific for p409/410 (FIG. 15A-C), MAb No. 9 immunohistochemistry (FIG. 15D-F) labeled all characteristic types of inclusions in ADNC cases with stage 3 LATE-NC in the amygdala (FIG. 15A and FIG. 15D), CA1 region (FIG. 15B and FIG. 15E), and frontal cortex (FIG. 15C and FIG. 15F), but MAb No. 9 exhibited a higher extent of TDP-43 pathology overall (FIG. 15D-F). Double-label immunofluorescence for MAb No. 9 (Cy3, orange-red) and p409/410 PAb (green) confirmed colocalization (yellow in merged image, arrow) (Pearson's coefficient value of 0.521), yet revealed additional TDP-43 positivity, indicated by fine red dot-like stains (arrowheads) in the merged image of the amygdala (FIG. 15G). The arrowhead indicates a TDP-43 Type β inclusion, while the rest of TDP-43 positivity represent TDP-43 Type α pathology. Nuclei stained with DAPI (blue) in merged images. Scale bar, 50 μm.

FIG. 16 shows an MSD assay measuring plasma EV TDP-43 levels from FTLD-TDP, FTLD-tau patients, and healthy aging controls (CON) with antibody pair 9-409/410.

FIG. 17 shows MSD assay using antibody pair 9-409/410 measuring TDP-43 levels in plasma before and after treatment with 2% SDS in FTLD-TDP patients, FTLD-tau patients, and healthy aging controls (CON). N=10.

FIG. 18 shows MSD assay using antibody pair 9-409/410 measuring TDP-43 levels in plasma after treatment with 2% SDS in FTLD-TDP patients, LATE-NC, FTLD-tau patients, and healthy aging controls (CON). N=20.

### DETAILED DESCRIPTION

Unless otherwise defined, all technical and scientific terms used herein have the same meaning as commonly understood by one of ordinary skill in the art. For example, any nomenclatures used in connection with, and techniques of biochemistry, molecular biology, immunology, microbiology, genetics, cell and tissue culture, and protein and nucleic acid chemistry described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms “amino acid,” “nucleotide,” “polynucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or Y) are used herein.

As used herein, terms such as “include,” “including,” “contain,” “containing,” “having,” and the like mean “comprising.” The present disclosure also contemplates other embodiments “comprising,” “consisting essentially of,” and “consisting of” the embodiments or elements presented herein, whether explicitly set forth or not. As used herein, “comprising,” is an “open-ended” term that does not exclude additional, unrecited elements or method steps. As used herein, “consisting essentially of” limits the scope of a claim to the specified materials or steps and those that do not materially affect the basic and novel characteristics of the claimed invention. As used herein, “consisting of” excludes any element, step, or ingredient not specified in the claim.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “and/or” refers to both the conjunctive and disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to ±10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “~” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to ±10% of any value within the range or within 3 or more standard deviations, including the end points, or as described above in the definition of “about.”

As used herein, the terms “room temperature,” “RT,” or “ambient temperature” refer to the typical temperature in an indoor laboratory setting. In one aspect, the laboratory setting is climate controlled to maintain the temperature at a substantially uniform temperature or with a specific range of temperatures. In one aspect, “room temperature” refers a temperature of about 15-30° C., including all integers and endpoints within the specified range. In another aspect, “room temperature” refers a temperature of about 15-30° C.; about 20-30° C.; about 22-30° C.; about 25-30° C.; about 27-30° C.; about 15-22° C.; about 15-25° C.; about 15-27° C.; about 20-22° C.; about 20-25° C.; about 20-27° C.; about 22-25° C.; about 22-27° C.; about 25-27° C.; about 15° C.±10%; about 20° C.±10%; about 22° C.±10%; about 25° C.±10%; about 27° C.±10%; ~20° C., ~22° C., ~25° C., or ~27° C., at standard atmospheric pressure.

The terms “control,” “reference level,” and “reference” are used herein interchangeably. The reference level may be a predetermined value or range, which is employed as a benchmark against which to assess the measured result. “Control group” as used herein refers to a group of control subjects or cells. A control may be a subject or cell without a synthetic peptide mimetic as detailed herein. A control may be a subject, or a sample therefrom, whose disease state is known. The subject, or sample therefrom, may be healthy, diseased, diseased prior to treatment, diseased during treatment, or diseased after treatment, or a combination thereof.

As used herein, the term “antibody” collectively refers to immunoglobulins or immunoglobulin-like molecules including by way of example and without limitation, IgA, IgD, IgE, IgG and IgM, combinations thereof, and similar molecules produced during an immune response in any vertebrate, for example, in mammals such as humans, goats, rabbits and mice, as well as non-mammalian species, such as shark immunoglobulins. The term “antibody” includes intact immunoglobulins and “antibody fragments” or “antigen binding fragments” that specifically bind to a molecule of interest (or a group of highly similar molecules of interest) to the substantial exclusion of binding to other molecules (for example, antibodies and antibody fragments that have a binding constant for the molecule of interest that is at least 10³ M⁻¹ greater, at least 10⁴ M⁻¹ greater, or at least 10⁵ M⁻¹ greater than a binding constant for other molecules in a biological sample). The term “antibody” also includes genetically engineered forms such as chimeric antibodies (for example, humanized murine antibodies), heteroconjugate antibodies (such as, bispecific antibodies). See also, Pierce Catalog and Handbook, 1994-1995 (Pierce Chemical Co., Rockford, III.); Kuby, Immunology, 3^{rd} ed., W.H. Freeman & Co., New York (1997). More particularly, “antibody” refers to a polypeptide ligand comprising at least a light chain or heavy chain immunoglobulin variable region which specifically recognizes and binds an epitope of an antigen. Antibodies are composed of a heavy and a light chain, each of which has a variable region, termed the variable heavy (VH) region and the variable light (VL) region. Together, the VH region and the VL region are responsible for binding the antigen recognized by the antibody.

Typically, an immunoglobulin has heavy (H) chains and light (L) chains interconnected by disulfide bonds. There are two types of light chain, lambda (λ) and kappa (κ). There are five main heavy chain classes (or isotypes) which determine the functional activity of an antibody molecule: IgM, IgD, IgG, IgA and IgE. Each heavy and light chain contains a constant region and a variable region, (the regions are also known as “domains”). In combination, the heavy and the light chain variable regions specifically bind the antigen. Light and heavy chain variable regions contain a “framework” region interrupted by three hypervariable regions, also called “complementarity-determining regions” or “CDRs.” The extent of the framework region and CDRs have been defined (see e.g., Kabat et al., Sequences of Proteins of Immunological Interest, U.S. Department of Health and Human Services, 1991, which is hereby incorporated by reference). The Kabat database is now maintained online. The sequences of the framework regions of different light or heavy chains are relatively conserved within a species. The framework region of an antibody, that is the combined framework regions of the constituent light and heavy chains, largely adopt a β-sheet conformation and the CDRs form loops which connect, and in some cases form part of, the β-sheet structure. Thus, framework regions act to form a scaffold that provides for positioning the CDRs in correct orientation by inter-chain, non-covalent interactions. The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a VH CDR3 is located in the variable domain of the heavy chain of the antibody in which it is found, whereas a VL CDR1 is the CDR1 from the variable domain of the light chain of the antibody in which it is found. An antibody that binds pathologic TDP-43 will have a specific VH region and the VL region sequence, and thus specific CDR sequences. Antibodies with different specificities (e.g., different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs).

The term “antibody” is further intended to encompass digestion fragments, specified portions, derivatives, and variants thereof, including antibody mimetics or comprising portions of antibodies that mimic the structure and/or function of an antibody or specified fragment or portion thereof, including single chain antibodies and fragments thereof. Examples of binding fragments encompassed within the term “antigen binding portion” of an antibody include a Fab fragment, a monovalent fragment consisting of the VL, VH, CL and CH, domains; a F(ab′)₂ fragment, a bivalent fragment comprising two Fab fragments linked by a disulfide bridge at the hinge region; a Fd fragment consisting of the VH and CH, domains; a Fv fragment consisting of the VL and VH domains of a single arm of an antibody, a dAb fragment, which consists of a VH domain; and an isolated complementarity determining region (CDR). See Ward et al. Nature 341:544-546 (1989). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv, or scFv). See e.g., Bird et al., Science 242:423-426 (1988); Huston et al., Proc. Natl. Acad Sci. USA 85:5879-5883 (1988). Single chain antibodies are also intended to be encompassed within the term “fragment of an antibody.” Any of the above-noted antibody fragments are obtained using conventional techniques known to those of skill in the art, and the fragments are screened for binding specificity and neutralization activity in the same manner as are intact antibodies.

“Antibody fragments” or “antigen binding fragments” include proteolytic antibody fragments (such as F(ab′)₂ fragments, Fab′ fragments, Fab′-SH fragments and Fab fragments as are known in the art), recombinant antibody fragments (such as sFᵥ fragments, dsFᵥ fragments, bispecific sFv fragments, bispecific dsFᵥ fragments, F (ab)′2 fragments, single chain Fᵥ proteins (“scFᵥ”), disulfide stabilized Fᵥ proteins (“dsFᵥ”), diabodies, and triabodies (as are known in the art), and camelid antibodies. See, e.g., U.S. Pat. Nos. 6,015,695; 6,005,079; 5,874,541; 5,840,526; 5,800,988; and 5,759,808. An scFᵥ protein is a fusion protein in which a light chain variable region of an immunoglobulin and a heavy chain variable region of an immunoglobulin are bound by a linker, while in dsFvs, the chains have been mutated to introduce a disulfide bond to stabilize the association of the chains.

As used herein, the term “antibody derivative” is intended to encompass molecules that bind an epitope as defined herein and which are modifications or derivatives of an isolated pathologic TDP-43-binding antibody of this disclosure. Derivatives include, but are not limited to, for example, bispecific, heterospecific, trispecific, tetraspecific, multispecific antibodies, diabodies, chimeric, recombinant, and humanized. As used herein, the term “bispecific molecule” is intended to include any agent, e.g., a protein, peptide, or protein or peptide complex, which has two different binding specificities. As used herein, the term “multispecific molecule” or “heterospecific molecule” is intended to include any agent, e.g., a protein, peptide, or protein or peptide complex, which has more than two different binding specificities. As used herein, the term “heteroantibodies” refers to two or more antibodies, antibody binding fragments (e.g., Fab), derivatives thereof, or antigen binding regions linked together, at least two of which have different specificities.

The term “antibody variant” is intended to include antibodies produced in a species other than a rabbit. It also includes antibodies containing post-translational modifications to the linear polypeptide sequence of the antibody or fragment. It further encompasses fully human antibodies.

As used herein, the term “antigen” refers to a compound, composition, or substance that may be specifically bound by the products of specific humoral or cellular immunity, such as an antibody molecule or T-cell receptor. Antigens can be any type of molecule including, for example, haptens, simple intermediary metabolites, sugars (e.g., oligosaccharides), lipids, and hormones as well as macromolecules such as complex carbohydrates (e.g., polysaccharides), phospholipids, peptides, oligopeptides, polypeptides, and proteins. Common categories of antigens include, but are not limited to, viral antigens, bacterial antigens, fungal antigens, protozoa and other parasitic antigens, antigens involved in neurological disease, tumor antigens, antigens involved in autoimmune disease, allergy and graft rejection, toxins, and other miscellaneous antigens.

As used herein, an “assay” or “diagnostic assay” can be of any type of assay applied in the field of diagnostics. Such an assay may be based on the binding of an analyte to be detected to one or more capture probes with a certain affinity.

“Binding Constants” are described herein. The term “association rate constant,” “Kₒₙ” or “kₐ” as used herein, refers to the value indicating the binding rate of an antibody to its target antigen or the rate of complex formation between an antibody and antigen as shown by the equation: Antibody (Ab)+Antigen (Ag)→Ab-Ag. In some embodiments disclosed herein, an antibody or antigen-binding fragment thereof may have a kₒₙ of at least about 1.0×10³ M⁻¹s⁻¹, at least about 1.0×10⁴ M⁻¹s⁻¹, at least about 5.0×10⁴ M⁻¹s⁻¹, at least about 1.0×10⁵ M⁻¹s⁻¹, at least about 2.0×10⁵ M⁻¹s⁻¹, at least about 3.0×10⁵ M⁻¹s⁻¹, at least about 4.0×10⁵ M⁻¹s⁻¹, at least about 5.0×10⁵ M⁻¹s⁻¹, least about 6.0×10⁵ M⁻¹s⁻¹, at least about 1.0×10⁶ M⁻¹s⁻¹, at least about 1.0×10⁷ M⁻¹s⁻¹, at least about 1.0×10⁸ M⁻¹s⁻¹, or a Kₒₙ ranging from about 1.0×10³ M⁻¹s⁻¹ to about 1.0×10⁸ M⁻¹s⁻¹, about 1.0×10⁴ M⁻¹s⁻¹ to about 1.0×10⁸ M⁻¹s⁻¹, about 1.0×10⁵ M⁻¹s⁻¹ to about 1.0×10⁸ M⁻¹s⁻¹, about 1.0×10⁶ M⁻¹s⁻¹ to about 1.0×10⁸ M⁻¹s⁻¹, about 1.0×10⁷ M⁻¹s⁻¹ to about 1.0×10⁸ M⁻¹s⁻¹, about 1.0×10³ M⁻¹s⁻¹ to about 1.0×10⁷ M⁻¹s⁻¹, about 1.0×10⁴ M⁻¹s⁻¹ to about 1.0×10⁷ M⁻¹s⁻¹, about 1.0×10⁵ M⁻¹s⁻¹ to about 1.0×10⁷ M⁻¹s⁻¹, about 1.0×10⁶ M⁻¹s⁻¹ to about 1.0×10⁷ M⁻¹s⁻¹, about 1.0×10⁴ M⁻¹s⁻¹ to about 1.0×10⁷ M⁻¹s⁻¹, about 1.0×10⁴ M⁻¹s⁻¹ to about 1.0×10⁶ M⁻¹s⁻¹, about 1.0×10⁴ M⁻¹s⁻¹ to about 1.0×10⁵ M⁻¹s⁻¹, about 1.0×10⁵ M⁻¹s⁻¹ to about 1.0×10⁷ M⁻¹s⁻¹, or about 1.0×10⁵ M⁻¹s⁻¹ to about 1.0×10⁶ M⁻¹s⁻¹.

The term “dissociation rate constant” or “K_{off}” as used interchangeably herein, refers to the value indicating the dissociation rate of an antibody form its target antigen or separation of Ab-Ag complex over time into free antibody and antigen as shown by the equation: Antibody (Ab)+Antigen (Ag)←Ab-Ag. In some embodiments disclosed herein, an antibody or antigen-binding fragment thereof may have a K_{off} of about 1.0×10⁻⁴ s⁻¹ or less, about 1.0×10⁻⁵ s⁻¹ or less, about 5.0×10⁻⁶ s⁻¹ or less, about 1.0×10⁻⁶ s⁻¹ or less, about 5.0×10⁻⁷ s⁻¹ or less, about 1.0×10⁻⁷ s⁻¹ or less, about 5.0×10⁻⁸ s⁻¹ or less, about 1.0×10⁻⁸ s⁻¹ or less, about 1.0×10⁻⁹ s⁻¹ or less, about 1.0×10⁻¹⁰ s⁻¹ or less, about 1.0×10⁻¹¹ s⁻¹ or less, about 1.0×10⁻¹² s⁻¹ or less, or a K_{off} ranging from about 1.0×10⁻¹² s⁻¹ to about 1.0×10⁻⁴ s⁻¹, about 1.0×10⁻¹² s⁻¹ to about 1.0×10⁻⁵ s⁻¹, about 1.0×10⁻¹² s⁻¹ to about 1.0×10⁻⁶ s⁻¹, about 1.0×10⁻¹² s⁻¹ to about 1.0×10⁻⁷ s⁻¹, about 1.0×10⁻¹² s⁻¹ to about 1.0×10⁻⁸ s⁻¹, about 1.0×10⁻¹² s⁻¹ to about 1.0×10⁻⁹ s⁻¹, about 1.0×10-12 s⁻¹ to about 1.0×10⁻¹⁰ s⁻¹, about 1.0×10⁻¹⁰ s⁻¹ to about 1.0×10⁻⁴ s⁻¹, about 1.0×10⁻¹⁰ s⁻¹ to about 1.0×10⁻⁵ s⁻¹, about 1.0×10⁻¹⁰ s⁻¹ to about 1.0×10⁻⁶ s⁻¹, about 1.0×10⁻¹⁰ s⁻¹ to about 1.0×10⁻⁷ s⁻¹, about 1.0×10⁻¹⁰ s⁻¹ to about 1.0×10⁻⁸ s⁻¹, about 1.0×10⁻⁸ s⁻¹ to about 1.0× 10-4 s⁻¹, about 1.0×10⁻⁸ s⁻¹ to about 1.0×10⁻⁵ s⁻¹, about 1.0×10⁻⁸ s⁻¹ to about 1.0×10⁻⁶ s⁻¹, about 1.0×10⁻⁸ s⁻¹ to about 1.0×10⁻⁷ s⁻¹, about 1.0×10⁻⁷ s⁻¹ to about 1.0×10⁻⁴ s⁻¹, about 1.0×10⁻⁷ s⁻¹ to about 1.0×10⁻⁵ s⁻¹, or about 1.0×10⁻⁷ s⁻¹ to about 1.0×10⁻⁶ s⁻¹.

Methods for determining association and dissociation rate constants are known in the art. Using fluorescence-based techniques offers high sensitivity and the ability to examine samples in physiological buffers at equilibrium. Other experimental approaches and instruments such as a BIACORE® (biomolecular interaction analysis) assay can be used (e.g., instrument available from BIAcore International AB, a GE Healthcare company, Uppsala, Sweden). Additionally, a KINEXA® (Kinetic Exclusion Assay) assay, available from Sapidyne Instruments (Boise, Idaho) can also be used.

The term “equilibrium dissociation constant,” “k_{d}” or “K_{D}” as used interchangeably herein, refers to the value obtained by dividing the dissociation rate (K_{off}) by the association rate (Kₒₙ). The association rate, the dissociation rate, and the equilibrium dissociation constant are used to represent the binding affinity of an antibody to an antigen.

As used herein, “binding affinity” refers to the tendency of one molecule to bind (typically non-covalently) with another molecule, such as the tendency of a member of a specific binding pair for another member of a specific binding pair. A binding affinity can be measured as a binding constant, which binding affinity for a specific binding pair (such as an antibody/antigen pair) can be at least 1×10⁻⁵ M, at least 1×10⁻⁶ M, at least 1×10⁻⁷ M, at least 1×10⁻⁸ M, at least 1×10⁻⁹ M, at least 1×10⁻¹⁰ M, at least 1×10⁻¹¹ M, or at least 1×10⁻¹² M. Binding affinity may be calculated by a modification of the Scatchard method described by Frankel et al., Mol. Immunol., 16:101-106, 1979 or by an antigen/antibody dissociation rate. A high binding affinity may be measured by a competition radioimmunoassay. A high binding affinity for an antibody/antigen pair may be at least about 1×10⁻⁸ M, at least about 1.5×10⁻⁸ M, at least about 2.0×10⁻⁸ M, at least about 2.5×10⁻⁸ M, at least about 3.0×10⁻⁸ M, at least about 3.5×10⁻⁸ M, at least about 4.0×10⁻⁸ M, at least about 4.5×10⁻⁸ M, or at least about 5.0×10⁻⁸ M.

“Binding region” or “target region” as used herein refers to the region within a target region of an antigen, and particularly an antigenic protein (e.g., TDP-43), that is recognized and bound by an antibody described herein.

As used herein, the term “biological equivalent thereof” is intended to be synonymous with “equivalent thereof” when referring to a reference protein, antibody, polypeptide, polynucleotide, or nucleic acid, and intends those having minimal homology while still maintaining desired structure or functionality. Unless specifically recited herein, it is contemplated that any nucleic acid, polynucleotide, polypeptide, protein, or antibody mentioned herein also includes equivalents thereof. For example, an equivalent intends at least about 80% homology or identity and alternatively, at least about 85%, or alternatively at least about 90%, or alternatively at least about 95%, or alternatively 98% percent homology or identity and exhibits substantially equivalent biological activity to the reference protein, polypeptide, antibody, or nucleic acid. In one aspect, the term “equivalent” or “biological equivalent” of an antibody means the ability of the antibody to selectively bind its epitope protein or fragment thereof as measured by ELISA, IHC, or other suitable methods. Biologically equivalent antibodies include, but are not limited to, those antibodies, peptides, antibody fragments, antibody variant, antibody derivative, and antibody mimetics that bind to the same epitope as the reference antibody. One skilled in the art can prepare an antibody functionally equivalent to the antibodies of the present disclosure by introducing appropriate mutations into the antibody using site-directed mutagenesis. See e.g., Hashimoto-Gotoh et al., Gene 152:271-275 (1995); Zoller & Smith, Met. Enzymol. 100:468-500 (1983); Kramer et al., Nucleic Acids Res. 12:9441-9456 (1984); Kramer et al., Met. Enzymol. 154:350-367 (1987); Kunkel, Proc. Natl. Acad. Sci. USA 82:488-492 (1985); and Kunkel, Met. Enzymol. 85:2763-2766 (1988). Antibodies that are functionally equivalent to the antibodies of the present disclosure and comprise an amino acid sequence comprising mutation of one or more amino acids in the amino acid sequence of an antibody of the present disclosure are also included in the antibodies of the present disclosure. In such mutants, the number of amino acids that are mutated may be generally 50 amino acids or less, preferably 30 or less, and more preferably 10 or less (for example, 5 amino acids or less). An amino acid residue may be mutated into one that conserves the properties of the amino acid side chain. For example, based on their side chain properties, amino acids are classified into: hydrophobic amino acids (A, I, L, M, F, P, W, Y, and V); hydrophilic amino acids (R, D, N, C, E, Q, G, H, K, S, and T); amino acids having aliphatic side-chains (G, A, V, L, I, and P); amino acids having hydroxyl group-containing side-chains (S, T, and Y); amino acids having sulfur atom-containing side-chains (C and M); amino acids having carboxylic acid- and amide-containing side-chains (D, N, E, and Q); base-containing side-chains (R, K, and H); and amino acids having aromatic-containing side-chains (H, F, Y, and W).

As used herein, the term “chimeric antibody” means an antibody in which the Fc constant region of a monoclonal antibody from one species (e.g., a mouse Fc constant region) is replaced, using recombinant DNA techniques, with an Fc constant region from an antibody of another species (e.g., a human Fc constant region). See e.g., U.S. Pat. No. 4,816,567; Int. Pat. App. Pub. Nos. WO 1987002671 A1, WO 198601533 A1; European Pat. App. Pub. Nos. EP 0184187 A2; EP 0171496 A2; EP 0173494 A2; EP 0125023 A1; Better et al., Science 240:1041-1043 (1988); Liu et al., Proc. Natl. Acad. Sci. USA 84:3439-3443 (1987); Liu et al., J. Immunol. 139:3521-3526 (1987); Sun et al., Proc. Natl. Acad. Sci. USA 84:214-218 (1987); Nishimura et al., Cancer Res. 47:999-1005 (1987); Wood et al., Nature 314:446-449 (1885); and Shaw et al., J. Natl. Cancer Inst. 80:1553-1559 (1988). For example, the target binding region or site may be from a non-human source (e.g. mouse or primate) and the constant region may be human.

“Variant” with respect to a peptide or polypeptide that differs in amino acid sequence by the insertion, deletion, or conservative substitution of amino acids, but retain at least one biological activity. Variant may also mean a protein with an amino acid sequence that is substantially identical to a referenced protein with an amino acid sequence that retains at least one biological activity. Representative examples of “biological activity” include the ability to be bound by a specific antibody or polypeptide or to promote a particular response. Variant can mean a functional fragment thereof. Variant can also mean multiple copies of a polypeptide. The multiple copies can be in tandem or separated by a linker. A conservative substitution of an amino acid, for example, replacing an amino acid with a different amino acid of similar properties (for example, hydrophilicity, degree and distribution of charged regions) is recognized in the art as typically involving a minor change. These minor changes may be identified, in part, by considering the hydropathic index of amino acids, as understood in the art. See Kyte et al., J. Mol. Biol. 157:105-132 (1982). The hydropathic index of an amino acid is based on a consideration of its hydrophobicity and charge. It is known in the art that amino acids of similar hydropathic indexes may be substituted and still retain protein function. In one aspect, amino acids having hydropathic indexes of ±2 are substituted. The hydrophilicity of amino acids may also be used to reveal substitutions that would result in proteins retaining biological function. A consideration of the hydrophilicity of amino acids in the context of a peptide permits calculation of the greatest local average hydrophilicity of that peptide. Substitutions may be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

As used herein, the term “detectable label” refers to a molecule or material that can produce a detectable (such as visually, electronically, or otherwise) signal that indicates the presence and/or concentration of the label in a sample. When conjugated to a specific binding molecule, the detectable label can be used to locate and/or quantify the target to which the specific binding molecule is directed. Thereby, the presence and/or concentration of the target in a sample can be detected by detecting the signal produced by the detectable label. A detectable label can be detected directly or indirectly, and several different detectable labels conjugated to different specific-binding molecules can be used in combination to detect one or more targets. For example, a first detectable label conjugated to an antibody specific to a target can be detected indirectly through the use of a second detectable label that is conjugated to a molecule that specifically binds the first detectable label. Multiple detectable labels that can be separately detected can be conjugated to different specific binding molecules that specifically bind different targets to provide a multiplexed assay that can provide simultaneous detection of the multiple targets in a sample. A detectable signal can be generated by any mechanism including absorption, emission and/or scattering of a photon (including radio frequency, microwave frequency, infrared frequency, visible frequency, and ultra-violet frequency photons). Detectable labels include colored, fluorescent, phosphorescent, and luminescent molecules and materials, catalysts (such as enzymes) that convert one substance into another substance to provide a detectable difference (such as by converting a colorless substance into a colored substance or vice versa, or by producing a precipitate or increasing sample turbidity), haptens that can be detected through antibody-hapten binding interactions using additional detectably labeled antibody conjugates, and paramagnetic and magnetic molecules or materials. Particular examples of detectable labels include enzymes such as horseradish peroxidase, alkaline phosphatase, acid phosphatase, glucose oxidase, β-galactosidase, or β-glucuronidase; fluorophores such as fluoresceins, luminophores, coumarins, BODIPY dyes, resorufins, and rhodamines (many additional examples of fluorescent molecules can be found in The Handbook—A Guide to Fluorescent Probes and Labeling Technologies, Molecular Probes, Eugene, Oreg.); nanoparticles such as quantum dots (obtained, for example, from QuantumDot Corp, Invitrogen Nanocrystal Technologies, Hayward, Calif.; see also, U.S. Pat. Nos. 6,815,064, 6,682,596, and 6,649,138, each of which is incorporated by reference herein); metal chelates such as DOTA and DPTA chelates of radioactive or paramagnetic metal ions like Gd3+; and liposomes, for example, liposomes containing trapped fluorescent molecules. Where the detectable label includes an enzyme, a detectable substrate such as a chromogen, a fluorogenic compound, or a luminogenic compound can be used in combination with the enzyme to generate a detectable signal (a wide variety of such compounds are commercially available, for example, from Invitrogen Corporation, Eugene, Oreg.). Particular examples of chromogenic compounds include diaminobenzidine (DAB), 4-nitrophenylphospate (pNPP), fast red, bromochloroindolyl phosphate (BCIP), nitro blue tetrazolium (NBT), BCIP/NBT, fast red, AP Orange, AP blue, tetramethylbenzidine (TMB), 2,2′-azino-di-[3-ethylbenzothiazoline sulphonate] (ABTS), o-dianisidine, 4-chloronaphthol (4-CN), nitrophenyl-β-D-galactopyranoside (ONPG), 0-phenylenediamine (OPD), 5-bromo-4-chloro-3-indolyl-β-galactopyranoside (X-Gal), methylumbelliferyl-β-D-galactopyranoside (MU-Gal), p-nitrophenyl-α-D-galactopyranoside (PNP), 5-bromo-4-chloro-3-indolyl-β-D-glucuronide (X-Gluc), 3-amino-9-ethyl carbazol (AEC), fuchsin, iodonitrotetrazolium (INT), tetrazolium blue, and tetrazolium violet. Alternatively, an enzyme can be used in a metallographic detection scheme. Metallographic detection methods include using an enzyme such as alkaline phosphatase in combination with a water-soluble metal ion and a redox-inactive substrate of the enzyme. The substrate is converted to a redox-active agent by the enzyme, and the redox-active agent reduces the metal ion, causing it to form a detectable precipitate. See, e.g., U.S. Pat. No. 7,642,064; Int. Pat. App. Pub. No. WO 2005/003777 A1, and U.S. Pat. App. Pub. No. US 2004/0265922 A1; each of which is incorporated by reference herein. Metallographic detection methods include using an oxido-reductase enzyme (such as horseradish peroxidase) along with a water-soluble metal ion, an oxidizing agent, and a reducing agent, again to form a detectable precipitate. See, for example, U.S. Pat. No. 6,670,113, which is incorporated by reference herein.

As used herein, an “epitope” or “antigenic determinant” refers to particular chemical groups or contiguous or non-contiguous peptide sequences on a molecule that an antibody binds to. An antibody may bind to a particular antigenic epitope. Epitopes usually consist of chemically active surface groupings of molecules such as amino acids or sugar side chains and usually have specific three-dimensional structural characteristics, as well as specific charge characteristics. Conformational and non-conformational epitopes are distinguished in that the binding to the former but not the latter is lost in the presence of denaturing solvents. An epitope may be unique to a misfolded protein. An epitope may be present in a misfolded protein and not in the native or normal conformation of the protein. In an embodiment, an antibody or fragment thereof as described herein may recognize an epitope in misfolded or pathologic TDP-43 and not in native or normal or non-pathologic TDP-43.

As used herein, “homology” or “identical”, percent “identity” or “similarity”, when used in the context of two or more nucleic acids or polypeptide sequences, refers to two or more sequences or subsequences that are the same or have a specified percentage of nucleotides or amino acid residues that are the same, e.g., at least 60%, 65%, 70%, 75%, 80%, 85%, 90%, 91%, 92%, 93%, 94%, 95%, 96%, 97%, 98%, 99%, or higher identity over a specified region (e.g., nucleotide sequence encoding an antibody described herein or amino acid sequence of an antibody described herein). Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. The alignment and the percent homology or sequence identity can be determined using software programs known in the art, for example those described in Current Protocols in Molecular Biology, Ausubel et al., eds. (1987) Supplement 30, section 7.7.18, Table 7.7.1. Default parameters may be used for alignment. For example, an alignment program is BLAST, using default parameters. In particular, preferred programs are BLASTN and BLASTP. The terms “homology,” “identical,” percent “identity,” or “similarity” also refer to, or can be applied to, the complement of a test sequence. The terms also include sequences that have deletions and/or additions, as well as those that have substitutions. As described herein, the preferred algorithms can account for gaps and the like. Preferably, identity exists over a region that is at least about 25 amino acids or nucleotides in length, or over a region that is at least 50-100 amino acids or nucleotides in length. An “unrelated” or “non-homologous” sequence shares less than 40% identity, or alternatively less than 25% identity, with one of the sequences of the present disclosure.

As used herein, the term “monoclonal antibody” or “MAb” refers to an antibody produced by a single clone of B-lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells (e.g., “hybridomas”). Monoclonal antibodies include humanized monoclonal antibodies.

As used herein, “neurodegenerative disease” refers to a group of conditions that result from progressive damage to neurons and nervous system connections that are essential for mobility, coordination, strength, sensation, and cognition. In particular, the neurodegenerative disease may be frontotemporal lobar degeneration (FTLD). “FTLD” refers to a group of disorders based on their neuropathology that cause damage and dysfunction of the frontal and temporal lobes of the brain. Many possible symptoms can result from FTLD, including, but not limited to, unusual behaviors, emotional problems, trouble communicating, difficulty with work, difficulty with walking, or combinations thereof. Alzheimer's disease (AD) is another neurodegenerative disorder that is a progressive deteriorative condition of the brain that affects memory, thought, and language. The degenerative changes of Alzheimer's disease lead to patches or plaques in the brain and the entanglement of nerve fibers (neurofibrillary tangles). Memory loss and behavioral changes occur as a result of these changes in brain tissue. AD, in contrast to FTLD, is usually a slow progressive illness that occurs in midlife but becomes increasingly more common over the age of 65 years. Difficulty with short-term memory is usually the first symptom of AD and early behavioral changes may not be noticed. As the disease progresses, memory loss increases and there are changes in personality, mood, and behavior. Disturbances of judgment and concentration occur, along with confusion and restlessness. The type, severity, sequence, and progression of mental changes vary widely. Long periods with little change are common, although occasionally the disease can be rapidly progressive. Alzheimer's disease generally affects most of the brain, whereas FTLD primarily affects the frontal and temporal lobes of the brain.

As used herein, “pathological” or “pathologic” pertains to or arises from disease. Pathological protein such as pathologic TDP-43 can be phosphorylated, be sensitive to phosphatases, have a different conformation from normal or non-pathological or healthy proteins, be misfolded, accumulate and form intranuclear and cytoplasmic aggregates.

A “peptide” or “polypeptide” is a linked sequence of two or more amino acids linked by peptide bonds. The polypeptide can be natural, synthetic, or a modification or combination of natural and synthetic. Peptides and polypeptides include proteins such as binding proteins, receptors, and antibodies. The terms “polypeptide,” “protein,” and “peptide” are used interchangeably herein. “Primary structure” refers to the amino acid sequence of a particular peptide. “Secondary structure” refers to locally ordered, three dimensional structures within a polypeptide. Certain structures within proteins are commonly referred to as domains, for example, enzymatic domains, extracellular domains, transmembrane domains, pore domains, and cytoplasmic tail domains. “Domains” are portions of a polypeptide that form a compact unit of the polypeptide and are typically 15 to 350 amino acids long. Exemplary domains include domains with enzymatic activity or ligand binding activity. Typical domains are made up of sections of lesser organization such as stretches of beta-sheet and alpha-helices. “Tertiary structure” refers to the complete three-dimensional structure of a polypeptide monomer. “Quaternary structure” refers to the three-dimensional structure formed by the noncovalent association of independent tertiary units. A “motif” is a portion of a polypeptide sequence and includes at least two amino acids. A motif may be 2 to 20, 2 to 15, or 2 to 10 amino acids in length. A motif may include 3, 4, 5, 6, or 7 sequential amino acids. A domain may be comprised of a series of the same type of motif.

“Proteinopathy,” e.g., any disease or condition that results from the abnormal synthesis, folding, post-translational modification, or deposition of protein in cells or tissues. Proteinopathy, as used herein, includes pathological proteins, such as pathological TDP-43 that can present epitopes not found in the TDP-43 protein in its native conformation.

“Sample” or “test sample” as used herein can mean any sample in which the presence and/or level of a target is to be detected or determined or any sample comprising an antibody or component thereof as detailed herein. Samples may include liquids, solutions, emulsions, or suspensions. Samples may include a medical sample. Samples may include any biological fluid or tissue, such as blood, whole blood, fractions of blood such as plasma and serum, muscle, interstitial fluid, sweat, saliva, urine, tears, synovial fluid, bone marrow, cerebrospinal fluid, nasal secretions, sputum, amniotic fluid, bronchoalveolar lavage fluid, gastric lavage, emesis, fecal matter, lung tissue, peripheral blood mononuclear cells, total white blood cells, lymph node cells, spleen cells, tonsil cells, cancer cells, tumor cells, bile, digestive fluid, skin, or combinations thereof. In some embodiments, the sample comprises an aliquot of a larger sample. In other embodiments, the sample comprises a biological fluid. Samples can be obtained by any means known in the art. The sample can be used directly as obtained from a patient or can be pretreated, such as by filtration, distillation, extraction, concentration, centrifugation, inactivation of interfering components, addition of reagents, and the like, to modify the character of the sample in some manner as discussed herein or otherwise as is known in the art.

“Sensitivity” and “specificity” as used herein with regard to a diagnostic and/or prognostic test depends on more than just the analytical “quality” of the test, they also depend on the definition of what constitutes an abnormal result. In practice, Receiver Operating Characteristic curves (ROC curves), are typically calculated by plotting the value of a variable versus its relative frequency in “normal” (e.g., apparently healthy individuals not having a particular disorder or condition) and “disease” populations. For any particular marker, a distribution of marker levels for subjects with and without a disease will likely overlap. Under such conditions, a test does not absolutely distinguish a normal condition from a disease condition with 100% accuracy, and the area of overlap indicates where the test cannot distinguish normal from disease. A threshold is selected, below which the test is considered to be abnormal and above which the test is considered to be normal, or vice versa. The area under the ROC curve is a measure of the probability that the perceived measurement will allow correct identification of a condition. ROC curves can be used even when test results do not necessarily give an accurate number. As long as one can rank results, one can create a ROC curve. For example, results of a test on “disease” samples might be ranked according to degree (e.g., 1=low, 2=normal, and 3=high). This ranking can be correlated to results in the “normal” population, and a ROC curve created. These methods are well known in the art. See e.g., Hanley et al., Radiology 143:29-36 (1982). For example, a threshold may be selected to provide a ROC curve area of greater than about 0.5, about 0.7, about 0.8, about 0.85, or about 0.9.

As used herein, a subject is “in need of treatment” if such subject would benefit biologically, medically, or in quality of life from such treatment. A subject in need of treatment does not necessarily present symptoms, particular in the case of preventative or prophylaxis treatments.

As used herein, the terms “active ingredient” or “active pharmaceutical ingredient” refer to a pharmaceutical agent, active ingredient, compound, or substance, compositions, or mixtures thereof, that provide a pharmacological, often beneficial, effect.

As used herein, the term “dose” denotes any form of an active ingredient formulation or composition, including cells, that contains an amount sufficient to initiate or produce a therapeutic effect with at least one or more administrations. “Formulation” and “composition” are used interchangeably herein.

As used herein, the term “prophylaxis” refers to preventing or reducing the progression of a disorder, either to a statistically significant degree or to a degree detectable by a person of ordinary skill in the art.

As used herein, the terms “effective amount” or “therapeutically effective amount,” refers to a substantially non-toxic, but sufficient amount of an action, agent, composition, or cell(s) being administered to a subject that will prevent, treat, or ameliorate to some extent one or more of the symptoms of the disease or condition being experienced or that the subject is susceptible to contracting. The result can be the reduction or alleviation of the signs, symptoms, or causes of a disease, or any other desired alteration of a biological system. An effective amount may be based on factors individual to each subject, including, but not limited to, the subject's age, size, type or extent of disease, stage of the disease, route of administration, the type or extent of supplemental therapy used, ongoing disease process, and type of treatment desired.

As used herein, the terms “subject” and “patient” are used interchangeably and refer to an animal. Typically, the subject is a mammal. A subject also refers to primates (e.g., humans, male or female; infant, adolescent, or adult), non-human primates, rats, mice, rabbits, pigs, cows, sheep, goats, horses, dogs, cats, fish, birds, and the like. In one embodiment, the subject is a primate. In one embodiment, the subject is a human. “Subject” and “patient” as used herein may also refer to any vertebrate, including, but not limited to, a mammal that wants or is in need of the herein described compositions or methods. The subject may be a human or a non-human. The subject may be a vertebrate. The subject may be a mammal. The mammal may be a primate or a non-primate. The mammal can be a non-primate such as, for example, cow, pig, camel, llama, hedgehog, anteater, platypus, elephant, alpaca, horse, goat, rabbit, sheep, hamster, guinea pig, cat, dog, rat, and mouse. The mammal can be a primate such as a human. The mammal can be a non-human primate such as, for example, monkey, cynomolgus monkey, rhesus monkey, chimpanzee, gorilla, orangutan, and gibbon. The subject may be of any age or stage of development, such as, for example, an adult, an adolescent, or an infant. The subject may be male or female. In some embodiments, the subject has a specific genetic marker. The subject may be undergoing treatment either for the disease or condition being diagnosed or for another disease or condition, or other forms of treatment.

As used herein, the terms “inhibit,” “inhibition,” or “inhibiting” refer to the reduction or suppression of a given biological process, condition, symptom, disorder, or disease, or a significant decrease in the baseline activity of a biological activity or process.

As used herein, “treatment” or “treating” refers to prophylaxis of, preventing, suppressing, repressing, reversing, alleviating, ameliorating, or inhibiting the progress of biological process including a disorder or disease, or completely eliminating a disease. A treatment may be either performed in an acute or chronic way. The term “treatment” also refers to reducing the severity of a disease or symptoms associated with such disease prior to affliction with the disease. “Repressing” or “ameliorating” a disease, disorder, or the symptoms thereof involves administering a cell, composition, or compound described herein to a subject after clinical appearance of such disease, disorder, or its symptoms. “Prophylaxis of” or “preventing” a disease, disorder, or the symptoms thereof involves administering a cell, composition, or compound described herein to a subject prior to onset of the disease, disorder, or the symptoms thereof. “Suppressing” a disease or disorder involves administering a cell, composition, or compound described herein to a subject after induction of the disease or disorder thereof but before its clinical appearance or symptoms thereof have manifest.

Fragments, derivatives, or analogs of the polypeptides of SEQ ID NO: 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, or 85 can be (i) ones in which one or more of the amino acid residues (e.g., 1, 2, 3, 4, 5, 7, 8, 9, 10, 11, 12, 13, 14, 15, 16, 17, 18, 19, 20, 25, 30, 35, 40, 45, or 50 residues, or even more) are substituted with a conserved or non-conserved amino acid residue (preferably a conserved amino acid residue). Such substituted amino acid residues may or may not be one encoded by the genetic code, or (ii) ones in which one or more of the amino acid residues includes a substituent group (e.g., 1, 2, 3, 4, 5, 7, 8, 9, 10, 15, 20, 25, 30, 35, 40, 45, or 50 residues or even more), or (iii) ones in which the mature polypeptide is fused with another polypeptide or compound, such as a compound to increase the half-life of the polypeptide (for example, polyethylene glycol), or (iv) ones in which the additional amino acids are fused to the mature polypeptide, such as an IgG Fc fusion region peptide or leader or secretory sequence or a sequence which is employed for purification of the mature polypeptide or a proprotein sequence. Such fragments, derivatives, and analogs are deemed to be within the scope of those skilled in the art from the teachings herein.

In addition, fragments, derivatives, or analogs of the polypeptides of SEQ ID NO: 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, or 85 can be substituted with one or more conserved or non-conserved amino acid residue (preferably a conserved amino acid residue). In some cases, these polypeptides, fragments, derivatives, or analogs thereof will have a polypeptide sequence at least 50%, 55%, 60%, 65%, 70%, 75%, 80%, 85%, 90%, 91%, 92%, 93%, 94%, 95%, 96%, 97%, 98%, 99% or 100% identical to the polypeptide sequence shown in SEQ ID NO: 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, or 85 and will comprise functional or non-functional proteins or enzymes. Similarly, additions or deletions to the polypeptides can be made either at the N- or C-termini or within non-conserved regions of the polypeptide (which are assumed to be non-critical because they have not been photogenically conserved).

As described herein, in many cases the amino acid substitutions, mutations, additions, or deletions are preferably of a minor nature, such as conservative amino acid substitutions that do not significantly affect the folding or activity of the protein or additions or deletions to the N- or C-termini. Of course, the number of amino acid substitutions, additions, or deletions a skilled artisan would make depends on many factors, including those described herein. Generally, the number of substitutions, additions, or deletions for any given polypeptide will not be more than about 100, 90, 80, 70, 60, 50, 40, 30, 25, 20, 19, 18, 17, 16, 15, 14, 13, 12, 11, 10, 9, 8, 7, 5, 6, 4, 3, 2, or 1.

The polypeptides described herein include encoding polynucleotides or variants that have substitutions, deletions, and/or additions that can involve one or more nucleotides. The variants can be altered in coding regions, non-coding regions, or both. Alterations in the coding regions can produce conservative or non-conservative amino acid substitutions, deletions, or additions. Especially preferred among these are silent substitutions, additions, and deletions, which do not alter the properties and activities of the binding.

Further embodiments described herein include nucleic acid molecules comprising polynucleotides having nucleotide sequences about 50%, 55%, 60%, 65%, 70%, 75%, 80%, 85%, 90%, 91%, 92%, 93%, 94%, 95%, 96%, 97%, 98%, or 99% identical, and more preferably at least about 90-99% or 100% identical to (a) nucleotide sequences, or degenerate, homologous, or codon-optimized variants thereof, encoding polypeptides having the amino acid sequences in SEQ ID NO: 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, or 85; (b) nucleotide sequences, or degenerate, homologous, or codon-optimized variants thereof, encoding polypeptides having the amino acid sequences in SEQ ID NO: 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, or 85; and (c) nucleotide sequences capable of hybridizing to the complement of any of the nucleotide sequences in (a) or (b) above and capable of expressing functional polypeptides of amino acid sequences in SEQ ID NO: 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, or 85.

By a polynucleotide having a nucleotide sequence at least, for example, 90-99% “identical” to a reference nucleotide sequence encoding a functional antibody binding domain or fragment thereof is intended that the nucleotide sequence of the polynucleotide be identical to the reference sequence except that the polynucleotide sequence can include up to about 10 to 1 point mutations, additions, or deletions per each 100 nucleotides of the reference nucleotide sequence encoding the functional antibody binding domain or fragment thereof.

In other words, to obtain a polynucleotide having a nucleotide sequence about at least 90-99% identical to a reference nucleotide sequence, up to 10% of the nucleotides in the reference sequence can be deleted, added, or substituted, with another nucleotide, or a number of nucleotides up to 10% of the total nucleotides in the reference sequence can be inserted into the reference sequence. These mutations of the reference sequence can occur at the 5′- or 3′-terminal positions of the reference nucleotide sequence or anywhere between those terminal positions, interspersed either individually among nucleotides in the reference sequence or in one or more contiguous groups within the reference sequence. The same is applicable to polypeptide sequences about at least 90-99% identical to a reference polypeptide sequence.

As noted above, two or more polynucleotide sequences can be compared by determining their percent identity. Two or more amino acid sequences likewise can be compared by determining their percent identity. The percent identity of two sequences, whether nucleic acid or peptide sequences, is generally described as the number of exact matches between two aligned sequences divided by the length of the shorter sequence and multiplied by 100. An approximate alignment for nucleic acid sequences is provided by the local homology algorithm of Smith and Waterman, Adv. App. Mathematics 2:482-489 (1981). This algorithm can be extended to use with peptide sequences using the scoring matrix developed by Dayhoff, Atlas of Protein Sequences and Structure, M. O. Dayhoff ed., Vol. 5 Suppl. 3:353-358 (1979), National Biomedical Research Foundation, Washington, D.C., USA, and normalized by Gribskov, Nucl. Acids Res. 14 (6): 6745-6763 (1986).

For example, due to the degeneracy of the genetic code, one having ordinary skill in the art will recognize that a large number of the nucleic acid molecules having a sequence at least 50%, 55%, 60%, 65%, 70%, 75%, 80%, 85%, 90%, 91%, 92%, 93%, 94%, 95%, 96%, 97%, 98%, 99%, or 100% identity to the nucleic acid sequence shown in SEQ ID NO: 5, 7, 9, 11, 13, 15, 17, 19, 21, 23, 25, 27, 29, 31, 33, 35, 37, 39, 41, 43, 45, 47, 49, 51, 53, 55, 57, 59, 61, 65, 67, 69, 71, 73, 75, 77, 79, 63, 81, or 83, or degenerate, homologous, or codon-optimized variants thereof, will encode a functional antibody binding domain or fragment thereof.

The polynucleotides described herein include those encoding mutations, variations, substitutions, additions, deletions, and particular examples of the polypeptides described herein. For example, guidance concerning how to make phenotypically silent amino acid substitutions is provided in Bowie et al., Science 247 (4948): 1306-1310 (1990), wherein the authors indicate that proteins are surprisingly tolerant of amino acid substitutions.

Another embodiment described herein is a polynucleotide vector comprising one or more nucleotide sequences described herein.

Another embodiment described herein is a cell comprising one or more nucleotide sequences described herein or a polynucleotide vector described herein.

Another embodiment is a polypeptide encoded by a nucleotide sequence described herein. In one aspect, the polypeptide has at least 85% to 99% identity, including all integers, end points and subranges within the specified range, to SEQ ID NO: 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, or 85. In another aspect, the polypeptide comprises a region having 100% identity to SEQ ID NO: 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, or 85.

Another embodiment described herein is a process for manufacturing one or more of the nucleotide sequence described herein or a polypeptide encoded by the nucleotide sequence described herein, the process comprising: transforming or transfecting a cell with a nucleic acid comprising a nucleotide sequence described herein; growing the cells; optionally isolating additional quantities of a nucleotide sequence described herein; inducing expression of a polypeptide encoded by a nucleotide sequence of described herein; isolating the polypeptide encoded by a nucleotide described herein.

Another embodiment described herein is a means for manufacturing one or more of the nucleotide sequences described herein or a polypeptide encoded by a nucleotide sequence described herein, the process comprising: transforming or transfecting a cell with a nucleic acid comprising a nucleotide sequence described herein; growing the cells; optionally isolating additional quantities of a nucleotide sequence described herein; inducing expression of a polypeptide encoded by a nucleotide sequence of described herein; isolating the polypeptide encoded by a nucleotide described herein.

Another embodiment described herein is a nucleotide sequence or a polypeptide encoded by the nucleotide sequence produced by the method or the means described herein

Another embodiment described herein is the use of an effective amount of a polypeptide encoded by one or more of the nucleotide sequences described herein in an antibody binding assay, method, or therapeutic application.

Another embodiment described herein is a research tool comprising a polypeptide encoded by a nucleotide sequence described herein.

Another embodiment described herein is a reagent comprising a polypeptide encoded by a nucleotide sequence described herein.

### Antibodies and Antigen-Binding Fragments Thereof

Provided herein are antibody or antigen-binding fragments thereof comprising an immunoglobulin heavy chain (HC) variable domain sequence and an immunoglobulin light chain (LC) variable domain sequence, wherein the heavy chain and light chain immunoglobulin variable domain sequences form an antigen binding site that binds to at least a portion of TAR DNA-binding protein 43 (TDP-43; SEQ ID NO: 2).

Also provided herein are antibody or antigen-binding fragments thereof comprising an immunoglobulin HC variable domain sequence and an immunoglobulin LC variable domain sequence, wherein the heavy chain and light chain immunoglobulin variable domain sequences form an antigen binding site that binds to at least a portion of the amino acid sequence of SEQ ID NO: 4.

Further provided herein are antibody or antigen-binding fragments thereof comprising an immunoglobulin heavy chain (HC) variable domain sequence and an immunoglobulin light chain (LC) variable domain sequence. The immunoglobulin HC variable domain sequence may comprise an HC CDR1 comprising the amino acid sequence of SEQ ID NO: 10, 30, 50, or 70; an HC CDR2 comprising the amino acid sequence of SEQ ID NO: 12, 32, 52, or 72; and an HC CDR3 comprising the amino acid sequence of SEQ ID NO: 14, 34, 54, or 74. The immunoglobulin LC variable domain sequence may comprise an LC CDR1 comprising the amino acid sequence of SEQ ID NO: 20, 40, 60, or 80; an LC CDR2 comprising the amino acid sequence of SEQ ID NO: 22, 42, 62, or 82; and an LC CDR3 comprising the amino acid sequence of SEQ ID NO: 24, 44, 64, or 84. The immunoglobulin HC variable domain sequence may comprise the amino acid sequence of SEQ ID NO: 8, 28, 48, or 68. The immunoglobulin LC variable domain sequence may comprise the amino acid sequence of SEQ ID NO: 18, 38, 58, or 78. The immunoglobulin HC domain sequence may comprise the amino acid sequence of SEQ ID NO: 6, 26, 46, or 66. The immunoglobulin LC domain sequence may comprise the amino acid sequence of SEQ ID NO: 16, 36, 56, or 76.

The antibodies as described herein may be a full-length antibody. The antibodies as described herein may be a monoclonal antibody. The antibodies described herein may comprise an Fc domain. The antibodies described herein may be a rabbit antibody, a human antibody, or a humanized antibody, or is non-immunogenic in a human. The antibodies as described herein may be a Fab, F (ab)′₂, Fab′, scFᵥ, or Fᵥ. One or more amino acid residues in a CDR of the antibodies or fragments thereof described herein may be substituted with another amino acid. The substitution may be conservative in the sense of being a substitution within the same family of amino acids.

### Immunoassays

Further provided herein are immunoassays comprising one or more of the above-described antibodies or antigen-binding fragments thereof. In some embodiments, the immunoassay may comprise one or more of the antibodies or antigen-binding fragments thereof as described herein. The immunoassay may be a sandwich immunoassay wherein two antibodies or antigen-binding fragments thereof as described herein are comprised by the immunoassay where one antibody or antigen-binding fragment thereof is a capture antibody, and a second antibody or antigen-binding fragment thereof is a detection antibody to form an immune complex. An immunoassay as described herein may be a radioimmunoassay (RIA), enzyme immunoassay (EIA), Enzyme-linked immunoassays (ELISA), immunohistochemistry (IHC), Meso Scale Discovery (MSD) biomarker assay (MesoScale, Inc., Rockville, Maryland), Western blotting, flow cytometry, counting immunoassay (CIA), fluoroimmunoassay (FIA), chemiluminescence immunoassay (CLIA), Luminex-based bead arrays (Luminex, Corp., Austin, TX), protein microarray assays, or rapid test formats such as immunochromatographic strip tests.

The immunoassays can be homogenous or heterogeneous assays, competitive and non-competitive assays. In a particular, the immunoassay may be in the form of a sandwich assay, which is a non-competitive immunoassay, wherein the molecule to be detected and/or quantified is bound to a first antibody and to a second antibody. The first antibody may be bound to a solid phase, e.g., a bead, a surface of a well or other container, a chip or a strip, and the second antibody is an antibody which is labeled, e.g., with a dye, with a radioisotope, or a reactive or catalytically active moiety or vice versa. The amount of labeled antibody bound to the analyte is then measured by an appropriate method. The general composition and procedures involved with “sandwich assays” are well-established and known to the skilled person (see e.g., The Immunoassay Handbook, Ed. David Wild, Elsevier LTD, Oxford; 3rd ed. (May 2005); Hultschig C et al., Curr Opin Chem Biol. 10 (1): 4-10 (2006), each of which is incorporated by reference herein for such teachings).

For example, the immunoassay may comprise two antibodies as described herein which are both present as dispersions in a liquid reaction mixture or one of the antibodies (e.g., the first antibody) may be labeled and the other antibody (e.g., the second antibody) may be bound to a solid phase or can be bound selectively to a solid phase wherein a first labelling component is attached to the first antibody, wherein said first labelling component is part of a labelling system based on fluorescence- or chemiluminescence-quenching or amplification, and a second labelling component of said marking system is attached to the second antibody, so that upon binding of both capture molecules to the analyte a measurable signal is generated that allows for the detection of the formed sandwich complexes in the solution comprising the sample.

Fluorescence based assays may comprise the use of dyes, which may be selected from the group comprising FAM (5- or 6-carboxyfluorescein), VIC, NED, Fluorescein, Fluorescein isothiocyanate (FITC), IRD-700/800, Cyanine dyes, such as CY3, CYS, CY3.5, CY5.5, Cy7, Xanthen, 6-Carboxy-2′,4′, 7′,4,7-hexachlorofluorescein (HEX), TET, 6-Carboxy-4′,5′-dichloro-2′,7′-dimethodyfluorescein (JOE), N,N,N′,N′-Tetramethyl-6-carboxyrhodamine (TAMRA), 6-Carboxy-X-rhodamine ((ROX), 5-Carboxyrhodamine-6G (R6G5), 6-carboxyrhodamine-6G (RG6), Rhodamine, Rhodamine Green, Rhodamine Red, Rhodamine 110, BODIPY dyes, such as BODIPY TMR, Oregon Green, Coumarins such as Umbelliferone, Benzamides, such as Hoechst 33258; Phenanthridines, such as Texas Red, Yakima Yellow, Alexa Fluor, PET, Ethidium bromide, Acridinium dyes, Carbazol dyes, Phenoxazine dyes, Porphyrin dyes, Polymethine dyes, and the like.

Chemiluminescence based assays may comprise the use of dyes, based on the physical principles described for chemiluminescent materials in Kirk-Othmer, Encyclopedia of Chemical Technology, 4^{th} ed., John Wiley & Sons, 15:518-562 (1993), incorporated herein by reference, including citations on pages 551-562. Chemiluminescent dyes may include acridinium esters, dioxetanes, or luminol.

### Methods of Detecting TDP-43 in a Biological Sample

TDP-43 is composed of 414 amino acids, and functions as a heterogeneous nuclear ribonucleoprotein. The recognized domains of TDP-43 include two highly conserved RNA-recognition motifs and a glycine-rich C-terminal domain. Under normal conditions, TDP-43 mainly localizes to the nucleus, and functions to regulate gene transcription and mRNA splicing. In FTLD-TDP cases, TDP-43 changes conformation, misfolds, and accumulates in intranuclear and cytoplasmic aggregates. These aggregates result in the loss of nuclear TDP-43 function and are themselves toxic. Depending on the distribution and relative abundance of these TDP-43 aggregates, FTLD-TDP can be further classified into four distinct histopathologic types, A-D. TDP-43 aggregates are predominantly composed of pathologic TDP-43 proteins: mainly 45 kDa phosphorylated full-length TDP-43 and 25 kDa C-terminal fragments. Notably, both the full-length and C-terminal fragments of TDP-43 are phosphatase-sensitive, indicating disease-associated hyperphosphorylation. TDP-43's phosphorylation sites are mostly located in the glycine-rich C-terminal domain of the protein.

Pathologic TDP-43 in plasma may be used as a biomarker for FTLD. A promising approach for developing a biomarker for FTLD is the quantification of disease-specific biochemical markers present in patients' biofluids, such as CSF and plasma. Brain-derived pathologic TDP-43 is a top candidate as a biomarker for FTLD. However, there are several hurdles that will have to be overcome to make pathologic TDP-43 a robust biomarker, including: it is challenging to measure pathologic TDP-43 in cerebrospinal fluid (CSF) and plasma samples since they also contain normal TDP-43; the level of brain-derived, pathologic TDP-43 is low in plasma because protein exchange between the brain and blood is highly regulated through the blood-brain barrier; the pathologic TDP-43 level may be slightly higher in CSF due to the direct contact of the CSF compartment with the brain; currently there are no antibodies specific for the pathologic form of TDP-43; to measure pathologic TDP-43 in patients' biofluids, nearly all reported studies have used antibodies that bind to phosphorylated epitopes at the C-terminus of TDP-43 and such antibodies likely detect both forms of pathologic TDP-43 (e.g., full-length and C-terminal fragments) as well as non-pathologic nuclear TDP-43 through the non-specific binding of phosphorylated epitopes. To address these hurdles, the antibodies described herein are highly sensitive and specific for the pathologic form of TDP-43 and the immunoassays described herein are highly sensitive and quantitative for detecting brain-derived pathologic TDP-43, while limiting the contribution of normal TDP-43 signals in biofluids obtained from subjects.

Provided herein are methods of detecting TDP-43 in a biological sample. The methods may comprise contacting a sample with an antibody or antigen-binding fragment thereof as described herein. The methods may further comprise contacting the biological sample with a second antibody or antigen-binding fragment thereof as described herein. The TDP-43 may be pathologic TDP-43, misfolded TDP-43, or a combination thereof. The sample may comprise a cell, tissue sample, or a biological fluid. The sample may be a plasma, serum, or CSF sample. The sample may be obtained from a subject that is diagnosed as having, suspected as having, or at risk of having or developing a neurodegenerative disease. The neurodegenerative disease may be frontotemporal lobar degeneration (FTLD) or frontotemporal dementia (FTD). Detecting TDP-43 may comprise an immunoassay as described herein.

### Methods of Diagnosing Neurodegenerative Diseases

Provided herein are methods of diagnosing a neurodegenerative disease in a subject. The method may comprise detecting the presence of TDP-43 in a biological sample from the subject as described herein. The neurodegenerative disease may be frontotemporal lobar degeneration (FTLD) or frontotemporal dementia (FTD).

### Methods of Diagnosing and Treating a Neurodegenerative Disease

Described herein are methods of diagnosing and treating a subject having a neurodegenerative disease. The methods may comprise detecting the presence of TDP-43 protein (e.g., pathologic or misfolded TDP-43) in a biological sample from the subject as described herein using one or more antibodies or antigen-binding fragments described herein. The neurodegenerative disease may be frontotemporal lobar degeneration (FTLD) or frontotemporal dementia (FTD). The methods may comprise treating the subject with one or more anti-neurodegenerative disease therapies. The anti-neurodegenerative disease therapies may comprise selective serotonin reuptake inhibitors (SSRIs), antipsychotics, speech and language therapy, physical and occupational therapy, Chimeric Antigen Receptor T-cell (CAR-T) therapy targeting neurons or microglia, Proteolysis-Targeting Chimera (PROTAC) therapy, or combinations thereof.

Methods of Isolating and Detecting Protein Biomarkers from Extracellular Vesicles

Described herein are methods for isolating and detecting a protein biomarker in a biological sample comprising extracellular vesicles (EVs). The method may comprise treating a sample with one or more solubilizing reagents to disrupt and lyse the EVs to release protein biomarkers for detection. In some aspects, the solubilizing reagent may comprise about 1-3% sodium dodecyl sulfate (SDS) detergent. In certain non-limiting exemplary aspects, the solubilizing reagent comprises 2% SDS detergent. In other exemplary aspects, the solubilizing reagent may comprise other lysis buffers such as, for example, Triton X-100, NP-40, RIPA, or Tween 20 buffer at concentrations ranging from about 0.5-3%. The sample may be treated with the solubilizing reagent at room temperature for a period of time of about 5 minutes to about 15 minutes (e.g., about 10 minutes). The method may further comprise one or more centrifugation and/or ultracentrifugation steps to enrich or isolate the EVs from the sample. The method may also further comprise subjecting the sample to one or more freeze-thaw cycles to help disrupt EVs and release the protein biomarker.

The method may further comprise detecting a presence or absence of a protein biomarker based on a measured binding or lack of binding of the sample to one or more antibodies or antigen-binding fragments thereof that are specific to the protein biomarker. Detecting may include performing one or more immunoassays using the one or more antibodies or antigen-binding fragments thereof specific to the protein biomarker. For example, detecting may comprise immunohistochemistry (IHC), Meso Scale Discovery (MSD) biomarker assay, Western blotting, flow cytometry, radioimmunoassay (RIA), counting immunoassay (CIA), enzyme immunoassays (EIA) or enzyme-linked immunosorbent assays (ELISA), sandwich ELISA, fluoroimmunoassay (FIA), chemiluminescence immunoassay (CLIA), or combinations thereof.

The biological sample comprising EVs may be a whole blood, serum, plasma, or cerebrospinal fluid (CSF) sample. In some aspects, an EV-derived protein biomarker may be associated with a disease or disorder. For example, the biological sample may be obtained from a subject that is diagnosed as having, suspected as having, or at risk of having or developing a neurodegenerative disease. In certain non-limiting exemplary aspects, the protein biomarker is a TAR DNA-binding protein 43 (TDP-43) protein or fragment thereof associated with a neurodegenerative disease including frontotemporal lobar degeneration (FTLD) or frontotemporal dementia (FTD).

These isolation methods allow for the detection of EV-based protein biomarkers from patients' biological samples (e.g., plasma samples) without the need to isolate the EVs using conventional methods such as ultracentrifugation, size-exclusion chromatography, precipitation, and microfluidics-based techniques, each of which has limitations in yield, specificity, and scalability.

Methods for Selecting Whether to Enroll a Subject in a Clinical Trial for Frontotemporal Lobar Degeneration with TAR DNA-Binding Protein 43 Inclusions (FTLD-TDP)

Provided herein are methods for selecting whether to enroll a subject in a clinical trial for FTLD-TDP. The method may include measuring expression levels of TDP-43 in a sample from the subject, wherein the measuring may comprise contacting the sample with one or more antibodies or antigen-binding fragments thereof as described herein. The method may further include comparing the expression levels of the TDP-43 to a threshold expression level. If the expression levels of TDP-43 are above the threshold expression level, the subject may be selected for the clinical trial. If the expression levels of TDP-43 are below the threshold expression level, the subject is not selected for the clinical trial.

### Kits

Also described herein are immunoassay kits, which may be used to perform any of the methods disclosed herein. In some embodiments, the kits comprise any one or more of the antibodies or antigen-binding fragments thereof described herein that are capable of binding to a TDP-43 protein or fragment thereof, and a detection reagent. The detection reagent may include, but is not limited to, enzymatic substrates, colorimetric substrates, chemiluminescent substrates, fluorescent substrates, radiolabeled substrates, or combinations thereof. In certain optional embodiments, the kits may further comprise one or more buffers and receptacles and/or one or more of packaging, labels, information, or instructions for use.

In some embodiments, the kits may further comprise one or more solid supports (e.g., beads) for an anti-TDP-43 antibody. The kit may also comprise a detection means. The detection means may include, but are not limited to, one or more of fluorescent, luminescent, radioactive, enzymatic, or colorimetric detection means. Non-limiting examples of detection means that may be used with the disclosed immunoassay kits include a fluorescence detection device for use in fluoroimmunoassays; a luminescent or chemiluminescent detection device for use in enzymatic detection assays such as ELISA, Western blotting, or immunohistochemistry; a Gamma or scintillation counter for use in radioimmunoassays; specific enzymes and reagents; and spectrophotometers and/or plate readers for use in colorimetric detection assays.

In certain embodiments, the use of the disclosed immunoassay kits will selectively detect a TDP-43 protein or fragment thereof in a biological sample. In certain embodiments, the use of the disclosed immunoassay kits will detect TDP-43 protein in a subject. In certain embodiments, the use of the disclosed immunoassay kits will determine a subject as having a neurodegenerative disease.

The kits may further comprise information, instructions, or both that use of the kit will provide diagnostic information for medical conditions in mammals (particularly humans). The information and instructions may be in the form of words, pictures, or both, and the like. Instructions included in kits may be affixed to packaging material or may be included as a package insert. While the instructions are typically written on printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD-ROM), and the like. As used herein, the term “instructions” may also include the address of an internet site that provides the instructions.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

Various embodiments and aspects of the inventions described herein are summarized by the following clauses:
Clause 1. An isolated antibody or antigen-binding fragment thereof capable of binding to a TAR DNA-binding protein 43 (TDP-43) protein or fragment thereof, the antibody or antigen-binding fragment thereof comprising an immunoglobulin heavy chain (HC) variable domain sequence and an immunoglobulin light chain (LC) variable domain sequence, wherein:
   the immunoglobulin HC variable domain sequence comprises:
      (i) an HC CDR1 comprising an amino acid sequence having at least 95-99% identity to SEQ ID NO: 10,
      (ii) an HC CDR2 comprising an amino acid sequence having at least 95-99% identity to SEQ ID NO: 12, and
      (iii) an HC CDR3 comprising an amino acid sequence having at least 95-99% identity to SEQ ID NO: 14; and
   the immunoglobulin LC variable domain sequence comprises:
      (i) an LC CDR1 comprising an amino acid sequence having at least 95-99% identity to SEQ ID NO: 20,
      (ii) an LC CDR2 comprising an amino acid sequence having at least 95-99% identity to SEQ ID NO: 22, and
      (iii) an LC CDR3 comprising an amino acid sequence having at least 95-99% identity to SEQ ID NO: 24;
   the immunoglobulin HC variable domain sequence comprises:
      (i) an HC CDR1 comprising an amino acid sequence having at least 95-99% identity to SEQ ID NO: 30,
      (ii) an HC CDR2 comprising an amino acid sequence having at least 95-99% identity to SEQ ID NO: 32, and
      (iii) an HC CDR3 comprising an amino acid sequence having at least 95-99% identity to SEQ ID NO: 34; and
   the immunoglobulin LC variable domain sequence comprises:
      (i) an LC CDR1 comprising an amino acid sequence having at least 95-99% identity to SEQ ID NO: 40,
      (ii) an LC CDR2 comprising an amino acid sequence having at least 95-99% identity to SEQ ID NO: 42, and
      (iii) an LC CDR3 comprising an amino acid sequence having at least 95-99% identity to SEQ ID NO: 44;
   the immunoglobulin HC variable domain sequence comprises:
      (i) an HC CDR1 comprising an amino acid sequence having at least 95-99% identity to SEQ ID NO: 50,
      (ii) an HC CDR2 comprising an amino acid sequence having at least 95-99% identity to SEQ ID NO: 52, and
      (iii) an HC CDR3 comprising an amino acid sequence having at least 95-99% identity to SEQ ID NO: 54; and
   the immunoglobulin LC variable domain sequence comprises:
      (i) an LC CDR1 comprising an amino acid sequence having at least 95-99% identity to SEQ ID NO: 60,
      (ii) an LC CDR2 comprising an amino acid sequence having at least 95-99% identity to SEQ ID NO: 62, and
      (iii) an LC CDR3 comprising an amino acid sequence having at least 95-99% identity to SEQ ID NO: 64; or
   the immunoglobulin HC variable domain sequence comprises:
      (i) an HC CDR1 comprising an amino acid sequence having at least 95-99% identity to SEQ ID NO: 70,
      (ii) an HC CDR2 comprising an amino acid sequence having at least 95-99% identity to SEQ ID NO: 72, and
      (iii) an HC CDR3 comprising an amino acid sequence having at least 95-99% identity to SEQ ID NO: 74; and
   the immunoglobulin LC variable domain sequence comprises:
      (i) an LC CDR1 comprising an amino acid sequence having at least 95-99% identity to SEQ ID NO: 80,
      (ii) an LC CDR2 comprising an amino acid sequence having at least 95-99% identity to SEQ ID NO: 82, and
      (iii) an LC CDR3 comprising an amino acid sequence having at least 95-99% identity to SEQ ID NO: 84.
Clause 2. The isolated antibody or antigen-binding fragment thereof of clause 1, wherein:
   the immunoglobulin HC variable domain sequence comprises:
      (i) an HC CDR1 comprising the amino acid sequence of SEQ ID NO: 10,
      (ii) an HC CDR2 comprising the amino acid sequence of SEQ ID NO: 12, and
      (iii) an HC CDR3 comprising the amino acid sequence of SEQ ID NO: 14; and
   the immunoglobulin LC variable domain sequence comprises:
      (i) an LC CDR1 comprising the amino acid sequence of SEQ ID NO: 20,
      (ii) an LC CDR2 comprising the amino acid sequence of SEQ ID NO: 22, and
      (iii) an LC CDR3 comprising the amino acid sequence of SEQ ID NO: 24;
   the immunoglobulin HC variable domain sequence comprises:
      (i) an HC CDR1 comprising the amino acid sequence of SEQ ID NO: 30,
      (ii) an HC CDR2 comprising the amino acid sequence of SEQ ID NO: 32, and
      (iii) an HC CDR3 comprising the amino acid sequence of SEQ ID NO: 34; and
   the immunoglobulin LC variable domain sequence comprises:
      (i) an LC CDR1 comprising the amino acid sequence of SEQ ID NO: 40,
      (ii) an LC CDR2 comprising the amino acid sequence of SEQ ID NO: 42, and
      (iii) an LC CDR3 comprising the amino acid sequence of SEQ ID NO: 44;
   the immunoglobulin HC variable domain sequence comprises:
      (i) an HC CDR1 comprising the amino acid sequence of SEQ ID NO: 50,
      (ii) an HC CDR2 comprising the amino acid sequence of SEQ ID NO: 52, and
      (iii) an HC CDR3 comprising the amino acid sequence of SEQ ID NO: 54; and
   the immunoglobulin LC variable domain sequence comprises:
      (i) an LC CDR1 comprising the amino acid sequence of SEQ ID NO: 60,
      (ii) an LC CDR2 comprising the amino acid sequence of SEQ ID NO: 62, and
      (iii) an LC CDR3 comprising the amino acid sequence of SEQ ID NO: 64; or
   the immunoglobulin HC variable domain sequence comprises:
      (i) an HC CDR1 comprising the amino acid sequence of SEQ ID NO: 70,
      (ii) an HC CDR2 comprising the amino acid sequence of SEQ ID NO: 72, and
      (iii) an HC CDR3 comprising the amino acid sequence of SEQ ID NO: 74; and
   the immunoglobulin LC variable domain sequence comprises:
      (i) an LC CDR1 comprising the amino acid sequence of SEQ ID NO: 80,
      (ii) an LC CDR2 comprising the amino acid sequence of SEQ ID NO: 82, and
      (iii) an LC CDR3 comprising the amino acid sequence of SEQ ID NO: 84.
Clause 3. The isolated antibody or antigen-binding fragment thereof of clause 1 or 2,
   wherein:
   the immunoglobulin HC variable domain sequence comprises an amino acid sequence having at least 95-99% identity to SEQ ID NO: 8, and the immunoglobulin LC variable domain sequence comprises an amino acid sequence having at least 95-99% identity to SEQ ID NO: 18;
   the immunoglobulin HC variable domain sequence comprises an amino acid sequence having at least 95-99% identity to SEQ ID NO: 28, and the immunoglobulin LC variable domain sequence comprises an amino acid sequence having at least 95-99% identity to SEQ ID NO: 38;
   the immunoglobulin HC variable domain sequence comprises an amino acid sequence having at least 95-99% identity to SEQ ID NO: 48, and the immunoglobulin LC variable domain sequence comprises an amino acid sequence having at least 95-99% identity to SEQ ID NO: 58; or
   the immunoglobulin HC variable domain sequence comprises an amino acid sequence having at least 95-99% identity to SEQ ID NO: 68, and the immunoglobulin LC variable domain sequence comprises an amino acid sequence having at least 95-99% identity to SEQ ID NO: 78.
Clause 4. The isolated antibody or antigen-binding fragment thereof of any one of clauses 1-3, wherein:
   the immunoglobulin HC variable domain sequence comprises the amino acid sequence of SEQ ID NO: 8, and the immunoglobulin LC variable domain sequence comprises the amino acid sequence of SEQ ID NO: 18;
   the immunoglobulin HC variable domain sequence comprises the amino acid sequence of SEQ ID NO: 28, and the immunoglobulin LC variable domain sequence comprises the amino acid sequence of SEQ ID NO: 38;
   the immunoglobulin HC variable domain sequence comprises the amino acid sequence of SEQ ID NO: 48, and the immunoglobulin LC variable domain sequence comprises the amino acid sequence of SEQ ID NO: 58; or
   the immunoglobulin HC variable domain sequence comprises the amino acid sequence of SEQ ID NO: 68, and the immunoglobulin LC variable domain sequence comprises the amino acid sequence of SEQ ID NO: 78.
Clause 5. The isolated antibody or antigen-binding fragment thereof of any one of clauses 1-4, wherein:
   the immunoglobulin HC variable domain sequence is encoded by a nucleic acid comprising the nucleotide sequence of SEQ ID NO: 7, and the immunoglobulin LC variable domain sequence is encoded by a nucleic acid comprising the nucleotide sequence of SEQ ID NO: 17;
   the immunoglobulin HC variable domain sequence is encoded by a nucleic acid comprising the nucleotide sequence of SEQ ID NO: 27, and the immunoglobulin LC variable domain sequence is encoded by a nucleic acid comprising the nucleotide sequence of SEQ ID NO: 37;
   the immunoglobulin HC variable domain sequence is encoded by a nucleic acid comprising the nucleotide sequence of SEQ ID NO: 47, and the immunoglobulin LC variable domain sequence is encoded by a nucleic acid comprising the nucleotide sequence of SEQ ID NO: 57; or
   the immunoglobulin HC variable domain sequence is encoded by a nucleic acid comprising the nucleotide sequence of SEQ ID NO: 67, and the immunoglobulin LC variable domain sequence is encoded by a nucleic acid comprising the nucleotide sequence of SEQ ID NO: 77.
Clause 6. The isolated antibody or antigen-binding fragment thereof of any one of clauses 1-5, wherein the isolated antibody or antigen-binding fragment thereof comprises:
   a heavy chain comprising an amino acid sequence having at least 95-99% identity to SEQ ID NO: 6, and a light chain comprising an amino acid sequence having at least 95-99% identity to SEQ ID NO: 16;
   a heavy chain comprising an amino acid sequence having at least 95-99% identity to SEQ ID NO: 26, and a light chain comprising an amino acid sequence having at least 95-99% identity to SEQ ID NO: 36;
   a heavy chain comprising an amino acid sequence having at least 95-99% identity to SEQ ID NO: 46, and a light chain comprising an amino acid sequence having at least 95-99% identity to SEQ ID NO: 56; or
   a heavy chain comprising an amino acid sequence having at least 95-99% identity to SEQ ID NO: 66, and a light chain comprising an amino acid sequence having at least 95-99% identity to SEQ ID NO: 76.
Clause 7. The isolated antibody or antigen-binding fragment thereof of any one of clauses 1-6, wherein the isolated antibody or antigen-binding fragment thereof comprises:
   a heavy chain comprising the amino acid sequence of SEQ ID NO: 6, and a light chain comprising the amino acid sequence of SEQ ID NO: 16;
   a heavy chain comprising the amino acid sequence of SEQ ID NO: 26, and a light chain comprising the amino acid sequence of SEQ ID NO: 36;
   a heavy chain comprising the amino acid sequence of SEQ ID NO: 46, and a light chain comprising the amino acid sequence of SEQ ID NO: 56; or
   a heavy chain comprising the amino acid sequence of SEQ ID NO: 66, and a light chain comprising the amino acid sequence of SEQ ID NO: 76.
Clause 8. The isolated antibody or antigen-binding fragment thereof of any one of clauses 1-7, wherein the isolated antibody or antigen-binding fragment thereof comprises:
   a heavy chain encoded by a nucleic acid comprising the nucleotide sequence of SEQ ID NO: 5, and a light chain encoded by a nucleic acid comprising the nucleotide sequence of SEQ ID NO: 15;
   a heavy chain encoded by a nucleic acid comprising the nucleotide sequence of SEQ ID NO: 25, and a light chain encoded by a nucleic acid comprising the nucleotide sequence of SEQ ID NO: 35;
   a heavy chain encoded by a nucleic acid comprising the nucleotide sequence of SEQ ID NO: 45, and a light chain encoded by a nucleic acid comprising the nucleotide sequence of SEQ ID NO: 55; or
   a heavy chain encoded by a nucleic acid comprising the nucleotide sequence of SEQ ID NO: 65, and a light chain encoded by a nucleic acid comprising the nucleotide sequence of SEQ ID NO: 75.
Clause 9. The isolated antibody or antigen-binding fragment thereof of any one of clauses 1-8, wherein the isolated antibody or antigen-binding fragment thereof immunospecifically binds to an epitope of a TDP-43 protein or fragment thereof.
Clause 10. The isolated antibody or antigen-binding fragment thereof of any one of clauses 1-9, wherein the isolated antibody or antigen-binding fragment thereof immunospecifically binds to at least a portion of the TDP-43 protein of SEQ ID NO: 2.
Clause 11. The isolated antibody or antigen-binding fragment thereof of any one of clauses 1-10, wherein the isolated antibody or antigen-binding fragment thereof immunospecifically binds to a pathologic TDP-43 protein or fragment thereof.
Clause 12. An isolated nucleic acid comprising a nucleotide sequence encoding the antibody or antigen-binding fragment thereof of any one of clauses 1-11.
Clause 13. A vector comprising the isolated nucleic acid of clause 12.
Clause 14. A cell comprising the vector of clause 13.
Clause 15. An immunoassay comprising one or more of the antibodies or antigen-binding fragments thereof of any one of clauses 1-11.
Clause 16. Use of one or more of the antibodies or antigen-binding fragments thereof of any one of clauses 1-11 to prescreen a subject for a clinical trial.
Clause 17. An immunoassay method for detecting TAR DNA-binding protein 43 (TDP-43) protein, the method comprising:
   contacting a biological sample suspected of having a TDP-43 protein or fragment thereof with one or more antibodies or antigen-binding fragments thereof capable of binding to a TDP-43 protein or fragment thereof, the antibodies or antigen-binding fragments thereof comprising an immunoglobulin heavy chain (HC) variable domain sequence and an immunoglobulin light chain (LC) variable domain sequence, wherein:
      the immunoglobulin HC variable domain sequence comprises:
         (i) an HC CDR1 comprising an amino acid sequence having at least 95-99% identity to SEQ ID NO: 10,
         (ii) an HC CDR2 comprising an amino acid sequence having at least 95-99% identity to SEQ ID NO: 12, and
         (iii) an HC CDR3 comprising an amino acid sequence having at least 95-99% identity to SEQ ID NO: 14; and
      the immunoglobulin LC variable domain sequence comprises:
         (i) an LC CDR1 comprising an amino acid sequence having at least 95-99% identity to SEQ ID NO: 20,
         (ii) an LC CDR2 comprising an amino acid sequence having at least 95-99% identity to SEQ ID NO: 22, and
         (iii) an LC CDR3 comprising an amino acid sequence having at least 95-99% identity to SEQ ID NO: 24;
      the immunoglobulin HC variable domain sequence comprises:
         (i) an HC CDR1 comprising an amino acid sequence having at least 95-99% identity to SEQ ID NO: 30,
         (ii) an HC CDR2 comprising an amino acid sequence having at least 95-99% identity to SEQ ID NO: 32, and
         (iii) an HC CDR3 comprising an amino acid sequence having at least 95-99% identity to SEQ ID NO: 34; and
      the immunoglobulin LC variable domain sequence comprises:
         (i) an LC CDR1 comprising an amino acid sequence having at least 95-99% identity to SEQ ID NO: 40,
         (ii) an LC CDR2 comprising an amino acid sequence having at least 95-99% identity to SEQ ID NO: 42, and
         (iii) an LC CDR3 comprising an amino acid sequence having at least 95-99% identity to SEQ ID NO: 44;
      the immunoglobulin HC variable domain sequence comprises:
         (i) an HC CDR1 comprising an amino acid sequence having at least 95-99% identity to SEQ ID NO: 50,
         (ii) an HC CDR2 comprising an amino acid sequence having at least 95-99% identity to SEQ ID NO: 52, and
         (iii) an HC CDR3 comprising an amino acid sequence having at least 95-99% identity to SEQ ID NO: 54; and
      the immunoglobulin LC variable domain sequence comprises:
         (i) an LC CDR1 comprising an amino acid sequence having at least 95-99% identity to SEQ ID NO: 60,
         (ii) an LC CDR2 comprising an amino acid sequence having at least 95-99% identity to SEQ ID NO: 62, and
         (iii) an LC CDR3 comprising an amino acid sequence having at least 95-99% identity to SEQ ID NO: 64; or
      the immunoglobulin HC variable domain sequence comprises:
         (i) an HC CDR1 comprising an amino acid sequence having at least 95-99% identity to SEQ ID NO: 70,
         (ii) an HC CDR2 comprising an amino acid sequence having at least 95-99% identity to SEQ ID NO: 72, and
         (iii) an HC CDR3 comprising an amino acid sequence having at least 95-99% identity to SEQ ID NO: 74; and
      the immunoglobulin LC variable domain sequence comprises:
         (i) an LC CDR1 comprising an amino acid sequence having at least 95-99% identity to SEQ ID NO: 80,
         (ii) an LC CDR2 comprising an amino acid sequence having at least 95-99% identity to SEQ ID NO: 82, and
         (iii) an LC CDR3 comprising an amino acid sequence having at least 95-99% identity to SEQ ID NO: 84; and
      detecting a presence or absence of TDP-43 protein in the sample based on a measured binding or lack of binding of the one or more antibodies or antigen-binding fragments thereof to the sample.
Clause 18. The method of clause 17, further comprising treating the sample with one or more solubilizing reagents.
Clause 19. The method of clause 18, wherein the one or more solubilizing reagents comprises 1-3% sodium dodecyl sulfate (SDS) detergent.
Clause 20. The method of any one of clauses 17-19, further comprising subjecting the sample to one or more freeze-thaw cycles.
Clause 21. The method of any one of clauses 17-20, wherein detecting comprises immunohistochemistry (IHC), Meso Scale Discovery (MSD) biomarker assay, Western blotting, flow cytometry, radioimmunoassay (RIA), counting immunoassay (CIA), enzyme immunoassays (EIA) or enzyme-linked immunosorbent assays (ELISA), sandwich ELISA, fluoroimmunoassay (FIA), chemiluminescence immunoassay (CLIA), or combinations thereof.
Clause 22. The method of any one of clauses 17-21, wherein the biological sample comprises whole blood, serum, plasma, or cerebrospinal fluid (CSF).
Clause 23. The method of any one of clauses 17-22, wherein the biological sample comprises a cell or a tissue sample.
Clause 24. The method of any one of clauses 17-23, wherein the biological sample comprises extracellular vesicles (EVs), and wherein at least a portion of detected TDP-43 protein is derived from EVs.
Clause 25. The method of any one of clauses 17-24, wherein the biological sample is obtained from a subject that is diagnosed as having, suspected as having, or at risk of having or developing a neurodegenerative disease.
Clause 26. The method of clause 25, wherein the neurodegenerative disease is frontotemporal lobar degeneration (FTLD).
Clause 27. The method of any one of clauses 17-26, wherein the TDP-43 protein is misfolded.
Clause 28. The method of any one of clauses 17-27, wherein the TDP-43 protein is a pathologic TDP-43 protein.
Clause 29. The method of any one of clauses 17-28, wherein one or more of the antibodies or antigen-binding fragments thereof are biotinylated.
Clause 30. The method of any one of clauses 17-29, further comprising contacting the sample with the detection peptide of SEQ ID NO: 85.
Clause 31. A method of detecting pathologic TAR DNA-binding protein 43 (TDP-43) protein in a subject, the method comprising:
   contacting a biological sample from the subject with one or more antibodies or antigen-binding fragments thereof capable of binding to a TDP-43 protein or fragment thereof, the antibodies or antigen-binding fragments thereof comprising an immunoglobulin heavy chain (HC) variable domain sequence and an
      immunoglobulin light chain (LC) variable domain sequence, wherein:
         (i) an HC CDR1 comprising an amino acid sequence having at least 95-99% identity to SEQ ID NO: 10,
         (ii) an HC CDR2 comprising an amino acid sequence having at least 95-99% identity to SEQ ID NO: 12, and
         (iii) an HC CDR3 comprising an amino acid sequence having at least 95-99% identity to SEQ ID NO: 14; and
      the immunoglobulin LC variable domain sequence comprises:
         (i) an LC CDR1 comprising an amino acid sequence having at least 95-99% identity to SEQ ID NO: 20,
         (ii) an LC CDR2 comprising an amino acid sequence having at least 95-99% identity to SEQ ID NO: 22, and
         (iii) an LC CDR3 comprising an amino acid sequence having at least 95-99% identity to SEQ ID NO: 24;
      the immunoglobulin HC variable domain sequence comprises:
         (i) an HC CDR1 comprising an amino acid sequence having at least 95-99% identity to SEQ ID NO: 30,
         (ii) an HC CDR2 comprising an amino acid sequence having at least 95-99% identity to SEQ ID NO: 32, and
         (iii) an HC CDR3 comprising an amino acid sequence having at least 95-99% identity to SEQ ID NO: 34; and
      the immunoglobulin LC variable domain sequence comprises:
         (i) an LC CDR1 comprising an amino acid sequence having at least 95-99% identity to SEQ ID NO: 40,
         (ii) an LC CDR2 comprising an amino acid sequence having at least 95-99% identity to SEQ ID NO: 42, and
         (iii) an LC CDR3 comprising an amino acid sequence having at least 95-99% identity to SEQ ID NO: 44;
      the immunoglobulin HC variable domain sequence comprises:
         (i) an HC CDR1 comprising an amino acid sequence having at least 95-99% identity to SEQ ID NO: 50,
         (ii) an HC CDR2 comprising an amino acid sequence having at least 95-99% identity to SEQ ID NO: 52, and
         (iii) an HC CDR3 comprising an amino acid sequence having at least 95-99% identity to SEQ ID NO: 54; and
      the immunoglobulin LC variable domain sequence comprises:
         (i) an LC CDR1 comprising an amino acid sequence having at least 95-99% identity to SEQ ID NO: 60,
         (ii) an LC CDR2 comprising an amino acid sequence having at least 95-99% identity to SEQ ID NO: 62, and
         (iii) an LC CDR3 comprising an amino acid sequence having at least 95-99% identity to SEQ ID NO: 64; or
      the immunoglobulin HC variable domain sequence comprises:
         (i) an HC CDR1 comprising an amino acid sequence having at least 95-99% identity to SEQ ID NO: 70,
         (ii) an HC CDR2 comprising an amino acid sequence having at least 95-99% identity to SEQ ID NO: 72, and
         (iii) an HC CDR3 comprising an amino acid sequence having at least 95-99% identity to SEQ ID NO: 74; and
      the immunoglobulin LC variable domain sequence comprises:
         (i) an LC CDR1 comprising an amino acid sequence having at least 95-99% identity to SEQ ID NO: 80,
         (ii) an LC CDR2 comprising an amino acid sequence having at least 95-99% identity to SEQ ID NO: 82, and
         (iii) an LC CDR3 comprising an amino acid sequence having at least 95-99% identity to SEQ ID NO: 84; and
   detecting a presence or absence of pathologic TDP-43 protein in the subject based on a measured binding or lack of binding of the one or more antibodies or antigen-binding fragments thereof to the sample.
Clause 32. The method of clause 31, wherein the pathologic TDP-43 protein is misfolded.
Clause 33. The method of clause 31 or 32, wherein the pathologic TDP-43 protein is phosphorylated at one or more residues.
Clause 34. The method of any one of clauses 31-33, wherein the subject has a neurodegenerative disease comprising frontotemporal lobar degeneration (FTLD).
Clause 35. A method of diagnosing and treating a subject having a neurodegenerative disease, the method comprising:
   contacting a sample from the subject with one or more antibodies or antigen-binding fragments thereof capable of binding to a TAR DNA-binding protein 43 (TDP-43) protein or fragment thereof, the antibodies or antigen-binding fragments thereof comprising an immunoglobulin heavy chain (HC) variable domain sequence and an immunoglobulin light chain (LC) variable domain sequence, wherein:
      the immunoglobulin HC variable domain sequence comprises:
         (i) an HC CDR1 comprising an amino acid sequence having at least 95-99% identity to SEQ ID NO: 10,
         (ii) an HC CDR2 comprising an amino acid sequence having at least 95-99% identity to SEQ ID NO: 12, and
         (iii) an HC CDR3 comprising an amino acid sequence having at least 95-99% identity to SEQ ID NO: 14; and
      the immunoglobulin LC variable domain sequence comprises:
         (i) an LC CDR1 comprising an amino acid sequence having at least 95-99% identity to SEQ ID NO: 20,
         (ii) an LC CDR2 comprising an amino acid sequence having at least 95-99% identity to SEQ ID NO: 22, and
         (iii) an LC CDR3 comprising an amino acid sequence having at least 95-99% identity to SEQ ID NO: 24;
      the immunoglobulin HC variable domain sequence comprises:
         (i) an HC CDR1 comprising an amino acid sequence having at least 95-99% identity to SEQ ID NO: 30,
         (ii) an HC CDR2 comprising an amino acid sequence having at least 95-99% identity to SEQ ID NO: 32, and
         (iii) an HC CDR3 comprising an amino acid sequence having at least 95-99% identity to SEQ ID NO: 34; and
      the immunoglobulin LC variable domain sequence comprises:
         (i) an LC CDR1 comprising an amino acid sequence having at least 95-99% identity to SEQ ID NO: 40,
         (ii) an LC CDR2 comprising an amino acid sequence having at least 95-99% identity to SEQ ID NO: 42, and
         (iii) an LC CDR3 comprising an amino acid sequence having at least 95-99% identity to SEQ ID NO: 44;
      the immunoglobulin HC variable domain sequence comprises:
         (i) an HC CDR1 comprising an amino acid sequence having at least 95-99% identity to SEQ ID NO: 50,
         (ii) an HC CDR2 comprising an amino acid sequence having at least 95-99% identity to SEQ ID NO: 52, and
         (iii) an HC CDR3 comprising an amino acid sequence having at least 95-99% identity to SEQ ID NO: 54; and
      the immunoglobulin LC variable domain sequence comprises:
         (i) an LC CDR1 comprising an amino acid sequence having at least 95-99% identity to SEQ ID NO: 60,
         (ii) an LC CDR2 comprising an amino acid sequence having at least 95-99% identity to SEQ ID NO: 62, and
         (iii) an LC CDR3 comprising an amino acid sequence having at least 95-99% identity to SEQ ID NO: 64; or
      the immunoglobulin HC variable domain sequence comprises:
         (i) an HC CDR1 comprising an amino acid sequence having at least 95-99% identity to SEQ ID NO: 70,
         (ii) an HC CDR2 comprising an amino acid sequence having at least 95-99% identity to SEQ ID NO: 72, and
         (iii) an HC CDR3 comprising an amino acid sequence having at least 95-99% identity to SEQ ID NO: 74; and
      the immunoglobulin LC variable domain sequence comprises:
         (i) an LC CDR1 comprising an amino acid sequence having at least 95-99% identity to SEQ ID NO: 80,
         (ii) an LC CDR2 comprising an amino acid sequence having at least 95-99% identity to SEQ ID NO: 82, and
         (iii) an LC CDR3 comprising an amino acid sequence having at least 95-99% identity to SEQ ID NO: 84;
   detecting a presence or absence of binding of the one or more antibodies or antigen-binding fragments thereof to the sample;
   diagnosing the subject with a neurodegenerative disease when antibody binding is detected; and
   treating the subject with one or more anti-neurodegenerative disease therapies.
Clause 36. The method of clause 35, wherein the sample comprises whole blood, serum, plasma, or cerebrospinal fluid (CSF).
Clause 37. The method of clause 35 or 36, wherein the neurodegenerative disease is frontotemporal lobar degeneration (FTLD).
Clause 38. The method of any one of clauses 35-37, wherein the one or more anti-neurodegenerative disease therapies comprises selective serotonin reuptake inhibitors (SSRIs), antipsychotics, speech and language therapy, physical and occupational therapy, Chimeric Antigen Receptor T-cell (CAR-T) therapy targeting neurons or microglia, Proteolysis-Targeting Chimera (PROTAC) therapy, or combinations thereof.
Clause 39. The method of any one of clauses 35-38, wherein the subject is a human.
Clause 40. A method for selecting whether to enroll a subject in a clinical trial for frontotemporal lobar degeneration with TAR DNA-binding protein 43 inclusions (FTLD-TDP), the method comprising:
   measuring protein expression levels of TAR DNA-binding protein 43 (TDP-43) in a sample from a subject, wherein the measuring comprises contacting the sample with one or more antibodies or antigen-binding fragments thereof capable of binding to a TDP-43 protein or fragment thereof, the antibodies or antigen-binding fragments thereof comprising an immunoglobulin heavy chain (HC) variable domain sequence and an immunoglobulin light chain (LC) variable domain sequence, wherein:
      the immunoglobulin HC variable domain sequence comprises:
         (i) an HC CDR1 comprising an amino acid sequence having at least 95-99% identity to SEQ ID NO: 10,
         (ii) an HC CDR2 comprising an amino acid sequence having at least 95-99% identity to SEQ ID NO: 12, and
         (iii) an HC CDR3 comprising an amino acid sequence having at least 95-99% identity to SEQ ID NO: 14; and
      the immunoglobulin LC variable domain sequence comprises:
         (i) an LC CDR1 comprising an amino acid sequence having at least 95-99% identity to SEQ ID NO: 20,
         (ii) an LC CDR2 comprising an amino acid sequence having at least 95-99% identity to SEQ ID NO: 22, and
         (iii) an LC CDR3 comprising an amino acid sequence having at least 95-99% identity to SEQ ID NO: 24;
      the immunoglobulin HC variable domain sequence comprises:
         (i) an HC CDR1 comprising an amino acid sequence having at least 95-99% identity to SEQ ID NO: 30,
         (ii) an HC CDR2 comprising an amino acid sequence having at least 95-99% identity to SEQ ID NO: 32, and
         (iii) an HC CDR3 comprising an amino acid sequence having at least 95-99% identity to SEQ ID NO: 34; and
      the immunoglobulin LC variable domain sequence comprises:
         (i) an LC CDR1 comprising an amino acid sequence having at least 95-99% identity to SEQ ID NO: 40,
         (ii) an LC CDR2 comprising an amino acid sequence having at least 95-99% identity to SEQ ID NO: 42, and
         (iii) an LC CDR3 comprising an amino acid sequence having at least 95-99% identity to SEQ ID NO: 44;
      the immunoglobulin HC variable domain sequence comprises:
         (i) an HC CDR1 comprising an amino acid sequence having at least 95-99% identity to SEQ ID NO: 50,
         (ii) an HC CDR2 comprising an amino acid sequence having at least 95-99% identity to SEQ ID NO: 52, and
         (iii) an HC CDR3 comprising an amino acid sequence having at least 95-99% identity to SEQ ID NO: 54; and
      the immunoglobulin LC variable domain sequence comprises:
         (i) an LC CDR1 comprising an amino acid sequence having at least 95-99% identity to SEQ ID NO: 60,
         (ii) an LC CDR2 comprising an amino acid sequence having at least 95-99% identity to SEQ ID NO: 62, and
         (iii) an LC CDR3 comprising an amino acid sequence having at least 95-99% identity to SEQ ID NO: 64; or
      the immunoglobulin HC variable domain sequence comprises:
         (i) an HC CDR1 comprising an amino acid sequence having at least 95-99% identity to SEQ ID NO: 70,
         (ii) an HC CDR2 comprising an amino acid sequence having at least 95-99% identity to SEQ ID NO: 72, and
         (iii) an HC CDR3 comprising an amino acid sequence having at least 95-99% identity to SEQ ID NO: 74; and
      the immunoglobulin LC variable domain sequence comprises:
         (i) an LC CDR1 comprising an amino acid sequence having at least 95-99% identity to SEQ ID NO: 80,
         (ii) an LC CDR2 comprising an amino acid sequence having at least 95-99% identity to SEQ ID NO: 82, and
         (iii) an LC CDR3 comprising an amino acid sequence having at least 95-99% identity to SEQ ID NO: 84;
   comparing measured protein expression levels of TDP-43 to a threshold TDP-43 expression level; and
   when the measured protein expression levels of TDP-43 are greater than the threshold TDP-43 expression level, selecting the subject for the clinical trial; or
   when the measured protein expression levels of TDP-43 are less than the threshold TDP-43 expression level, not selecting the subject for the clinical trial.
Clause 41. A method for isolating and detecting a protein biomarker in a biological sample comprising extracellular vesicles (EVs), the method comprising:
   treating the sample with a solubilizing reagent comprising about 1-3% sodium dodecyl sulfate (SDS) detergent to lyse the EVs; and
   detecting a presence or absence of the protein biomarker based on a measured binding or lack of binding of the sample to one or more antibodies or antigen-binding fragments thereof that are specific to the protein biomarker.
Clause 42. The method of clause 41, wherein the sample is treated with the solubilizing reagent at room temperature for a period of time of about 5 minutes to about 15 minutes. The method of clause 41 or 42, further comprising centrifuging the sample. Clause 43.
Clause 44. The method of any one of clauses 41-43, further comprising subjecting the sample to one or more freeze-thaw cycles.
Clause 45. The method of any one of clauses 41-44, wherein the biological sample comprises whole blood, serum, plasma, or cerebrospinal fluid (CSF).
Clause 46. The method of any one of clauses 41-45, wherein the biological sample is obtained from a subject that is diagnosed as having, suspected as having, or at risk of having or developing a neurodegenerative disease.
Clause 47. The method of any one of clauses 41-46, wherein detecting comprises immunohistochemistry (IHC), Meso Scale Discovery (MSD) biomarker assay, Western blotting, flow cytometry, radioimmunoassay (RIA), counting immunoassay (CIA), enzyme immunoassays (EIA) or enzyme-linked immunosorbent assays (ELISA), sandwich ELISA, fluoroimmunoassay (FIA), chemiluminescence immunoassay (CLIA), or combinations thereof.
Clause 48. The method of any one of clauses 41-47, wherein the protein biomarker is a TAR DNA-binding protein 43 (TDP-43) protein or fragment thereof.
Clause 49. The method of clause 48, wherein detecting comprises contacting the sample with one or more antibodies or antigen-binding fragments thereof capable of binding to a TDP-43 protein or fragment thereof, the antibodies or antigen-binding fragments thereof comprising an immunoglobulin heavy chain (HC) variable domain sequence and an immunoglobulin light chain (LC) variable domain sequence, wherein:
   the immunoglobulin HC variable domain sequence comprises:
      (i) an HC CDR1 comprising an amino acid sequence having at least 95-99% identity to SEQ ID NO: 10,
      (ii) an HC CDR2 comprising an amino acid sequence having at least 95-99% identity to SEQ ID NO: 12, and
      (iii) an HC CDR3 comprising an amino acid sequence having at least 95-99% identity to SEQ ID NO: 14; and
   the immunoglobulin LC variable domain sequence comprises:
      (i) an LC CDR1 comprising an amino acid sequence having at least 95-99% identity to SEQ ID NO: 20,
      (ii) an LC CDR2 comprising an amino acid sequence having at least 95-99% identity to SEQ ID NO: 22, and
      (iii) an LC CDR3 comprising an amino acid sequence having at least 95-99% identity to SEQ ID NO: 24;
   the immunoglobulin HC variable domain sequence comprises:
      (i) an HC CDR1 comprising an amino acid sequence having at least 95-99% identity to SEQ ID NO: 30,
      (ii) an HC CDR2 comprising an amino acid sequence having at least 95-99% identity to SEQ ID NO: 32, and
      (iii) an HC CDR3 comprising an amino acid sequence having at least 95-99% identity to SEQ ID NO: 34; and
   the immunoglobulin LC variable domain sequence comprises:
      (i) an LC CDR1 comprising an amino acid sequence having at least 95-99% identity to SEQ ID NO: 40,
      (ii) an LC CDR2 comprising an amino acid sequence having at least 95-99% identity to SEQ ID NO: 42, and
      (iii) an LC CDR3 comprising an amino acid sequence having at least 95-99% identity to SEQ ID NO: 44;
   the immunoglobulin HC variable domain sequence comprises:
      (i) an HC CDR1 comprising an amino acid sequence having at least 95-99% identity to SEQ ID NO: 50,
      (ii) an HC CDR2 comprising an amino acid sequence having at least 95-99% identity to SEQ ID NO: 52, and
      (iii) an HC CDR3 comprising an amino acid sequence having at least 95-99% identity to SEQ ID NO: 54; and
   the immunoglobulin LC variable domain sequence comprises:
      (i) an LC CDR1 comprising an amino acid sequence having at least 95-99% identity to SEQ ID NO: 60,
      (ii) an LC CDR2 comprising an amino acid sequence having at least 95-99% identity to SEQ ID NO: 62, and
      (iii) an LC CDR3 comprising an amino acid sequence having at least 95-99% identity to SEQ ID NO: 64; or
   the immunoglobulin HC variable domain sequence comprises:
      (i) an HC CDR1 comprising an amino acid sequence having at least 95-99% identity to SEQ ID NO: 70,
      (ii) an HC CDR2 comprising an amino acid sequence having at least 95-99% identity to SEQ ID NO: 72, and
      (iii) an HC CDR3 comprising an amino acid sequence having at least 95-99% identity to SEQ ID NO: 74; and
   the immunoglobulin LC variable domain sequence comprises:
      (i) an LC CDR1 comprising an amino acid sequence having at least 95-99% identity to SEQ ID NO: 80,
      (ii) an LC CDR2 comprising an amino acid sequence having at least 95-99% identity to SEQ ID NO: 82, and
      (iii) an LC CDR3 comprising an amino acid sequence having at least 95-99% identity to SEQ ID NO: 84.
Clause 50. An immunoassay kit for selectively detecting a TAR DNA-binding protein 43 (TDP-43) protein or fragment thereof in a biological sample, the kit comprising:
   one or more antibodies or antigen-binding fragments thereof capable of binding to a TDP-43 protein or fragment thereof, the antibodies or antigen-binding fragments thereof comprising an immunoglobulin heavy chain (HC) variable domain sequence and an immunoglobulin light chain (LC) variable domain sequence, wherein:
      the immunoglobulin HC variable domain sequence comprises:
         (i) an HC CDR1 comprising an amino acid sequence having at least 95-99% identity to SEQ ID NO: 10,
         (ii) an HC CDR2 comprising an amino acid sequence having at least 95-99% identity to SEQ ID NO: 12, and
         (iii) an HC CDR3 comprising an amino acid sequence having at least 95-99% identity to SEQ ID NO: 14; and
      the immunoglobulin LC variable domain sequence comprises:
         (i) an LC CDR1 comprising an amino acid sequence having at least 95-99% identity to SEQ ID NO: 20,
         (ii) an LC CDR2 comprising an amino acid sequence having at least 95-99% identity to SEQ ID NO: 22, and
         (iii) an LC CDR3 comprising an amino acid sequence having at least 95-99% identity to SEQ ID NO: 24;
      the immunoglobulin HC variable domain sequence comprises:
         (i) an HC CDR1 comprising an amino acid sequence having at least 95-99% identity to SEQ ID NO: 30,
         (ii) an HC CDR2 comprising an amino acid sequence having at least 95-99% identity to SEQ ID NO: 32, and
         (iii) an HC CDR3 comprising an amino acid sequence having at least 95-99% identity to SEQ ID NO: 34; and
      the immunoglobulin LC variable domain sequence comprises:
         (i) an LC CDR1 comprising an amino acid sequence having at least 95-99% identity to SEQ ID NO: 40,
         (ii) an LC CDR2 comprising an amino acid sequence having at least 95-99% identity to SEQ ID NO: 42, and
         (iii) an LC CDR3 comprising an amino acid sequence having at least 95-99% identity to SEQ ID NO: 44;
      the immunoglobulin HC variable domain sequence comprises:
         (i) an HC CDR1 comprising an amino acid sequence having at least 95-99% identity to SEQ ID NO: 50,
         (ii) an HC CDR2 comprising an amino acid sequence having at least 95-99% identity to SEQ ID NO: 52, and
         (iii) an HC CDR3 comprising an amino acid sequence having at least 95-99% identity to SEQ ID NO: 54; and
      the immunoglobulin LC variable domain sequence comprises:
         (i) an LC CDR1 comprising an amino acid sequence having at least 95-99% identity to SEQ ID NO: 60,
         (ii) an LC CDR2 comprising an amino acid sequence having at least 95-99% identity to SEQ ID NO: 62, and
         (iii) an LC CDR3 comprising an amino acid sequence having at least 95-99% identity to SEQ ID NO: 64; or
      the immunoglobulin HC variable domain sequence comprises:
         (i) an HC CDR1 comprising an amino acid sequence having at least 95-99% identity to SEQ ID NO: 70,
         (ii) an HC CDR2 comprising an amino acid sequence having at least 95-99% identity to SEQ ID NO: 72, and
         (iii) an HC CDR3 comprising an amino acid sequence having at least 95-99% identity to SEQ ID NO: 74; and
      the immunoglobulin LC variable domain sequence comprises:
         (i) an LC CDR1 comprising an amino acid sequence having at least 95-99% identity to SEQ ID NO: 80,
         (ii) an LC CDR2 comprising an amino acid sequence having at least 95-99% identity to SEQ ID NO: 82, and
         (iii) an LC CDR3 comprising an amino acid sequence having at least 95-99% identity to SEQ ID NO: 84;
   a detection reagent;
   optionally, buffers and receptacles; and
   optionally, one or more of packaging or instruction for use.
Clause 51. The kit of clause 50, further comprising a solid support for the one or more antibodies or antigen-binding fragments thereof.
Clause 52. The kit of clause 50 or 51, further comprising a detection means.
Clause 53. The kit of clause 52, wherein the detection means is one or more of fluorescent, luminescent, radioactive, or colorimetric.
Clause 54. The kit of any one of clauses 50-53, wherein the detection reagent comprises colorimetric substrates, chemiluminescent substrates, fluorescent substrates, or combinations thereof.
Clause 55. Use of one or more antibodies or antigen-binding fragments thereof of any one of clauses 1-11 in an immunoassay for detecting TAR DNA-binding protein 43 (TDP-43) protein in a biological sample.
Clause 56. Use of one or more antibodies or antigen-binding fragments thereof of any one of clauses 1-11 in a method of determining a subject as having a neurodegenerative disease.

### EXAMPLES

### Example 1

Subjects. This study will include plasma (n=50 per group) and CSF (n=30 per group) samples from neuropathologically confirmed FTLD-TDP, FTLD-tau, and AD patients, and from normal aging individuals. The plasma and CSF samples have been obtained from the brain banks of the Alzheimer's Disease Centers (ADCs) of Northwestern University, the University of Pittsburgh, and the University of Pennsylvania. The inclusion and exclusion criteria are listed in Table 1.

TABLE 1
Inclusion and exclusion criteria.

| Group | Inclusion Criteria | Exclusion Criteria |
|---|---|---|
| FTLD-TDP | Subjects meet the pathology | Any of the following: |
| | diagnostic guideline for FTLD-TDP. See e.g., | 1. Subjects meet the 2012 National |
| | Mackenzie et al., Acta Neuropathol. | Institute on Aging and Alzheimer's |
| | 117(1): 15-8 (2009); Cairns et al., Acta | Association (NIA-AA) diagnostic |
| | Neuropathol. 114(1): 5-22 (2007); | guideline for moderate or high AD |
| | Mackenzie et al., Acta Neuropathol. | neuropathologic change. See Hyman et al., |
| | 119(1): 1-4 (2010); McKhann et al., | Alzheimers Dement. 8(1): 1-13 (2012); |
| | Arch Neurol. 58(11): 1803-1809 (2001). | Montine et al., Acta Neuropathol. |
| | | 123(1): 1-11 (2012). |
| | | 2. Subjects meet the pathology |
| | | diagnostic guideline for FTLD-tau. See e.g., |
| | | Mackenzie et al., Acta Neuropathol. |
| | | 117(1): 15-8 (2009); Cairns et al., Acta |
| | | Neuropathol. 114(1): 5-22 (2007); |
| | | Mackenzie et al., Acta Neuropathol. |
| | | 119(1):1-4 (2010); McKhann et al., Arch |
| | | Neurol. 58(11): 1803-1809 (2001). |
| | | 3. Subjects have stroke history with |
| | | pathology confirmation. |
| FTLD-tau | Subjects meet the pathology | Any of the following: |
| | diagnostic guideline for FTLD-tau. See e.g., | 1. Subjects meet the 2012 NIA-AA |
| | Mackenzie et al., Acta Neuropathol. | diagnostic guideline for moderate or |
| | 117(1):15-8 (2009); Cairns et al., Acta | high AD neuropathologic change. |
| | Neuropathol. 114(1): 5-22 (2007); | 2. Subjects meet the pathology |
| | Mackenzie et al., Acta Neuropathol. | diagnostic guideline for FTLD-TDP. See e.g., |
| | 119(1):1-4 (2010); McKhann et al., | (Mackenzie et al., Acta Neuropathol. |
| | Arch Neurol. 58(11): 1803-1809 (2001). | 117(1): 15-8 (2009); Cairns et al., Acta |
| | | Neuropathol. 114(1): 5-22 (2007); |
| | | Mackenzie et al., Acta Neuropathol. |
| | | 119(1):1-4 (2010); McKhann et al., Arch |
| | | Neurol. 58(11): 1803-1809 (2001). |
| | | 3. Subjects have stroke history with |
| | | pathology confirmation. |
| AD | Subjects meet the 2012 NIA-AA | Any of the following: |
| | diagnostic guideline for moderate or | 1. Subjects meet the pathology |
| | high AD neuropathologic change. | diagnostic guideline for FTLD-TDP and |
| | See Hyman et al., Alzheimers Dement. | FTLD-tau. See e.g., Mackenzie et al., Acta |
| | 8(1): 1-13 (2012); Montine et al., Acta | Neuropathol. 117(1): 15-8 (2009); Cairns |
| | Neuropathol. 123(1): 1-11 (2012). | et al., Acta Neuropathol. |
| | | 114(1): 5-22 (2007); Mackenzie et al., |
| | | Acta Neuropathol. 119(1): 1-4 (2010); |
| | | McKhann et al., Arch Neurol. |
| | | 58(11): 1803-1809 (2001). |
| | | 2. Subjects have stroke history with |
| | | pathology confirmation. |
| Control | Age >40 years. | 1. Any subjects with infarct, |
| | Subjects who received lumbar | hemorrhage, epilepsy, tumor, |
| | puncture for exclusion diagnostics, | traumatic brain injury, or other |
| | where a negative finding was | neurodegenerative disease. |
| | obtained. | |

ELISA for Pathologic TDP-43 levels, and statistical analysis plan and sample size consideration. Plasma/CSF pathologic TDP-43 levels will be measured by an ELISA system described herein, with strict performance acceptance criteria and quality control samples on each plate. With each assay, the clinical samples will be processed together with a blank solution (sample diluent) and the (prepared) calibrator solutions. Low, medium, and high TDP-43 concentration quality control samples will be run on each assay plate, with an acceptance coefficient of variance (CV) cutoff of ≤20%. All samples will be run in triplicate. TDP-43 replicates with a CV>20% will be excluded from the analyses. All reported concentrations should fall within the qualified range of the assays. TDP-43 concentrations will be determined on a standard curve by plotting optical density versus concentration, using four-parameter logistic curve-fitting. Plasma/CSF data will be presented using box-plots, and potential outliers will be carefully examined and removed if necessary. Differences between plasma/CSF TDP-43 levels of FTLD-TDP patients with those of each of the other groups, e.g., FTLD-tau patients, AD patients, and control individuals, will be evaluated by two-sample t-test. Although the outcome might not follow normal distribution, a t-test is still expected to be appropriate based on Central Limit Theorem and the proposed sample size (n=50 for plasma and n=30 for CSF). To address the multiple testing issue, Bonferroni's adjustment will be applied. A receiver operating characteristic (ROC) curve will be plotted using plasma/CSF TDP-43 levels to diagnose of FTLD-TDP, but with different threshold criteria. The areas under the curve (AUC) for plasma and CSF TDP-43 levels will be compared. Fifty subjects would result in 80% statistical power to detect an AUC of 0.68 at a one-sided p<0.05 while assuming the actual AUC is 0.86. Similar ROC analyses will also be performed between FTLD-TDP, FTLD-tau, AD, and the aging control group to show the differences in TDP-43 levels between patients and controls. Statistical analyses will be performed using SAS 9.4. The Pearson's Correlation between CSF and plasma TDP-43 levels will be directly estimated with its 95% confidence interval.

### Production of Novel MAbs Against TDP-43

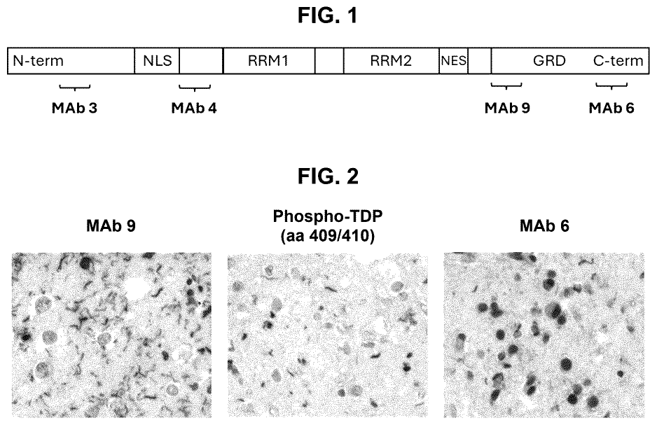

New TDP-43 MAbs were generated for the development of a disease-specific biomarker of FTLD-TDP. More than 3,000 Mab clones were screened by indirect ELISA and 13 promising MAbs were identified, which were then subjected to further epitope mapping by Western blotting. The epitope regions of four of those antibodies, illustrated in FIG. 1, are the N-terminus for MAb No. 3 (AA 25-50) and No. 4 (AA 98-120) and the glycine-rich C-terminal domain for MAb No. 6 (AA 390-410) and No. 9 (AA 280-300).

### MAb Immunoreactivity in Human Tissues

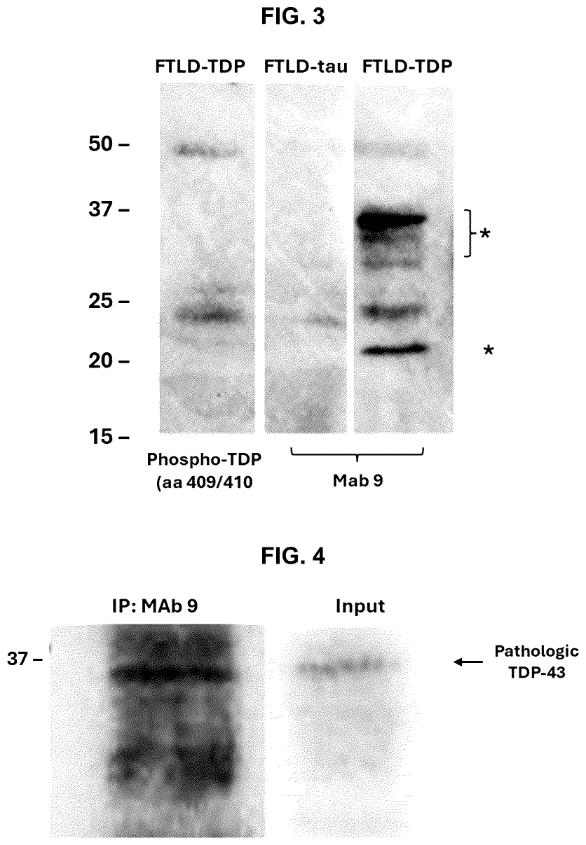

The antibodies described above were tested in the brain tissue slices obtained from FTLD-TDP patients. MAb No. 3 and No. 4 showed robust immunoreactivity for normal nuclear TDP-43 and minimal to weak reactivity for pathologic inclusions. MAb No. 6 was immunoreactive to both normal nuclear TDP-43 and pathologic inclusions. MAb No. 9 was strongly immunoreactive to all forms of pathological TDP-43 inclusions, with minimal reactivity for normal TDP-43 (FIG. 2). Immunoblotting analyses of urea soluble extracts from human FTLD-TDP brain tissues showed that MAb No. 3 and No. 4 recognized full-length TDP-43 but not the C-terminal fragments of TDP-43, while MAb No. 6 recognized both. Moreover, MAb No. 3 and No. 4 also did not detect the pathologic hyperphosphorylated 45 kDa band nor the high molecular weight smears, which were detected by MAb No. 6. Notably, MAb No. 9 only showed reactivity to pathologic TDP-43 species, with minimal reactivity to normal full-length TDP-43, which indicates that it is specific to pathologic TDP-43. Immunoblotting analyses of CSF from FTLD-TDP patients with MAb No. 9 showed similar results (FIG. 3).

### MAb No. 9 is Specific for Pathologic TDP-43

MAb No. 9 is unique from all reported MAbs that are specific for C-terminal TDP-43. Consistently until now, studies have shown that all C-terminal TDP-43-specific antibodies are immunoreactive not only to pathologic TDP-43 but also to normal nuclear TDP-43. See Feneberg et al., Mol. Neurobiol. 55 (10): 7789-7801 (2018). However, MAb No. 9 as described herein displayed high reactivity to pathologic TDP-43 inclusions, in the absence of significant reactivity to normal nuclear TDP-43 (FIG. 2). The underlying reason for this phenomenon is unknown, but it is likely due to the fact that MAb No. 9 recognizes an epitope that is only selectively exposed in pathologic TDP-43. This ability of MAb No. 9 to preferentially detect pathologic TDP-43 inclusions, with minimal or no normal nuclear TDP-43 immunohistochemical staining, is similar to the staining pattern seen with pathologic phospho-TDP-43-specific MAbs, such as the p409/410 MAb. However, MAb No. 9 appeared even more sensitive and was able to reveal a greater degree of pathology in different brain regions of FTLD-TDP patients compared to p409/410 MAb (FIG. 2). In addition, the TDP-43 pathology load revealed by MAb No. 9 is closely related to the severity of local neuronal loss, which was not reliably seen in studies using the p409/410 MAb. Interestingly, immunoblotting analyses of CSF samples from FTLD-TDP patients with MAb No. 9 revealed strong 20 kDa and 30-35 kDa fragments, which are rarely observed in brain tissues of human FTLD-TDP patients, in addition to the 45 kDa and 25 kDa fragments that were also revealed by p409/410 MAb (FIG. 3).

### ELISA Studies

ELISA systems were established to detect both normal and pathologic TDP-43 with the MAbs described herein. For normal full-length TDP-43, first antibodies were screened for the best pair and then a sandwich ELISA was established using MAb No. 3 and No. 4 as the capture and detection antibody, respectively. This platform showed that the lower limit of detection for human recombinant rTDP-43 (OriGene Technologies, Rockville, MD) was 25 pg/mL. Using this sensitive ELISA, the amount of TDP-43 in HEK293 cell lysates and supernatant was able to be quantified. Then, a sandwich ELISA was set up using MAb No. 9 and No. 6 as the capture and detection antibody, respectively, for detecting pathologic TDP-43. Using this sensitive ELISA, the amount of TDP-43 in the CSF samples from human FTLD-TDP patients was quantified, with a lower limit of detection of 60 pg/mL. Thus, these ELISA systems, and especially the ELISA for detecting pathologic TDP-43, represent new and valuable tools for biomarker discovery and investigation, tools that may accelerate the diagnosis of FTLD-TDP, and may be useful for the screening and selection of FTLD-TDP patients for inclusion in clinical trials.

Verification of TDP-43 ELISA System with Tandem Mass Spectrometry

Tandem mass spectrometry (MS)-based proteomic analysis will be performed to: determine amino acid sequence of the pathologic form of TDP-43 detected by MAb No. 9; perform large-scale relative quantitative proteomic analysis of pathologic TDP-43 across all 50 plasma and 30 CSF samples from FTLD-Tau, FTLD-TDP-43, AD, and age matched controls; and quantify the absolute level of pathologic TDP-43 in a subset of the plasma and CSF samples. For the experiment aimed to determine the relative quantification of plasma and CSF TDP-43 it is expected that compelling discovery-based quantitative results showing that all or nearly all the FTLD-TDP-43 cases have significantly elevated levels of pathologic TDP-43 will be obtained.

Preliminary data showed that MAb No. 9 was useful for immunoprecipitation (IP) for pathologic TDP-43 (FIG. 4). The TDP-43 IP will be further optimized using MAb No. 9. The recovery of TDP-43 will be assessed with Western blots (WBs) and the purity will be assessed with silver staining. Large batches of MAb No. 9 will be coupled to Dynabeads M-270 Epoxy beads (e.g., 2.8 μm superparamagnetic beads with surface exposed epoxy groups). By covalently coupling the primary amino and sulfhydryl groups in antibodies to the beads, the amount of IgG protein present in the purified material will be limited. The efficiency of antibody/bead coupling will be assessed by SDS-PAGE using silver staining and WB. In these initial experiments the amount of antibody and input plasma/CSF for robust purification of pathologic TDP-43 will also be optimized. With this knowledge in hand, pathologic TDP-43 will be immunoprecipitated with MAb No. 9 from plasma and CSF and the purified proteins will be resolved with SDS-PAGE. Next, a gel slice will be excised at 30-45 kDa to reduce sample complexity and increase the likelihood that mass spectrometry can characterize the complete amino acid composition of pathologic TDP-43. To increase sequence coverage of TDP-43, parallel in-gel digestion with several proteases including trypsin, Lys-C, Glu-C, Asp-N, and Lys-N will be performed.

The peptides will be separated, analyzed, identified, and mapped to reference TDP-43 amino acid sequence from Uniprot or NCBI. Savas et al., Science 335 (6071): 942 (2012); Jha et al., Nature 546 (7660): 651-655 (2017). Briefly, the peptides will be autosampler-loaded onto a C18 vented trap/analytical column assembly and separated with a Rapid Separation Liquid Chromatography (RSLC) ultra-high-pressure nano-flow liquid chromatography system (Thermo Fisher, Waltham, MA). The intact peptides will be electro-sprayed into the gas phase by applying a voltage to a stainless-steel emitter tip directly into a Orbitrap Fusion Tribrid mass spectrometer. To analyze the proteomic data, ProLuCID. Xu et al., J. Proteomics 129:16-24 (2015), DTASelect, Tabb et al., J. Proteome Res. 1 (1): 21-26 (2002); Census, Park et al., Bioinformatics 30 (15): 2208-2209 (2014); Park et al., Nature Methods 5:319-322 (2008) bioinformatic analysis software within the IP2 environment (integratedproteomics.com) will be used. To further interrogate the data, Skyline (MacCoss Lab, University of Washington) will be used. These software packages will be used and allow for identification of peptides and proteins and control the 1% false discovery rate based on the target decoy strategy. These software packages also allow for quantification of peptide abundance relative to the heavy-labeled internal standards based on the area under the curve from reconstructed chromatograms.

In the next experiment the reliability of the ELISA results will be tested with unbiased MS analysis. In these experiments a leading analytical workflow based on isobaric tandem mass tags (TMT) and multinotch MS3 analysis will be used. He et al., Mol. Psychiatry 24 (11): 1732-1747 (2019). Specifically, large-scale relative quantitative proteomic analysis of pathologic TDP-43 levels across all 50 plasma and 30 CSF samples from FTLD-Tau, FTLD-TDP-43, AD, and age matched controls will be performed. The goal here is to confirm the ELISA based clear results that can easily differentiate the FTLD-TDP samples from the others based on elevated levels of pathologic TDP-43.

Each patient derived sample (plasma or CSF) will be subjected to IP with MAb No. 9 using the conditions determined above. The purified material will be denatured with 6 M guanidine hydrochloride, reduced, alkylated, and digested to peptides with trypsin and LysC proteases. The peptides will be purified with reverse phase resin and chemically labeled with one TMT tags. 16plex TMT reagent (Thermo Fisher, Waltham, MA) will be used and in each MS analysis run 4 FTLD-Tau, 4 FTLD-TDP, 4 AD, and 4 age matched controls will be combined. In total at least 13× 16-plex experiments for plasma and 8×16-plex experiments for CSF will be performed. The peptides will be identified, and relative quantification will be performed with the bioinformatic analysis software described above.

In the final experiment, the absolute abundance of pathologic TDP-43 will be measured in a subset of the plasma and CSF samples. The goal here is to obtain orthogonal MS based measures to compare and confirm those obtained with ELISA. While the precise measures obtained with MS may not be highly correlated to those from ELISA, it is expected that the measures and the trends will be similar.

The first step will be to affinity purify pathologic TDP-43 with MAb No. 9 and digest the purified material with trypsin. Next, at least 3 chemically synthesized SpikeTide TQL peptides containing a single heavy C-terminal arginine residue (JPT Peptide Technologies GmbH, Berlin, Germany) will be spiked in. SpikeTide TQL peptides are strategic since they are quantified using a proprietary Quanti-Tag. Peptides are released from tag by tryptic digestion and aliquoted at 0.5 nM. These peptides will be spiked into the peptide mixtures at 5 different concentrations and purified with C18 Ziptips in to obtain a standard curve. The purified peptide concentrated using a SpeedVac vacuum concentrator (Labconco Corporation, Kansas City, MO), and analyzed by LC-MS/MS with a 2- or 4-hour analysis runs with an Orbitrap Fusion MS. The resulting spectra will be extracted, searched, and quantified with Prolucid/Sequest DTASelect and Census. The reconstructed MS1 chromatograms (area under the curves relative to the know amount of heavy peptide spiked into the sample) were used to determine the absolute peptide quantities.

It is anticipated that the proposed studies will objectively cross-verify the data from the ELISA. The quality of the CSF/plasma samples may vary depending on the patient's end stage condition; hence, the CSF/plasma samples will be cleaned up by immunoprecipitation using the specific MAb against pathologic TDP-43.

The ELISA system described herein will be transformed into a multiplex, Meso Scale Discovery (MSD)-based immunoassay (Meso Scale Diagnostics, LLC, Rockville, MD) that also includes other major dementia biomarkers, such as AD signature markers (Aβ42, T-tau, and P-tau181P), PGRN, and neurofilament light (NfL) subunit, for the diagnosis and follow-up of dementia-related diseases. Using MSD-based immunoassay, the sensitivity of a previous PGRN ELISA was increased from 60 ng/ml to 9 μg/mL. In addition to higher sensitivity, an MSD-based immunoassay also has the advantage of being quicker, requiring less sample volume to run, as well as having better reproducibility and precision. See e.g., Constantine et al., J. Virol. Meth. 47 (1-2): 153-164 (1994); Kuhle et al., Clin. Chem. Lab. Med. 54 (10): 1655-1661 (2016); Tatebe et al., Mol. Neurodegener. 12 (1): 63 (2017); Kuhle et al., Mult. Scler. 22 (12): 1550-1559 (2016).

### Determination of Whether Plasma Pathologic TDP-43 Levels Reflect the Severity of Pathology in FTLD-TDP Brains

Brain pathology will be quantified by measuring neuronal loss and gliosis and TDP-43 pathology burden, and this will elucidate the correlation between severity of brain pathology and plasma pathologic TDP-43 levels in FTLD-TDP patients. In addition to the plasma and CSF specimens of the above Examples, hematoxylin & eosin (H&E) slides and paraffin blocks of brain tissue from different brain regions from patients with FTLD-TDP will be obtained from the brain banks and analyzed.

It will be determined if plasma and CSF pathologic TDP-43 is a sensitive and specific biomarker for disease progression in FTLD-TDP. Using the novel ELISA described herein, the pathologic TDP-43 levels will be measured in the plasma/CSF of FTLD-TDP patients at early, middle, and late stages, and it will be determined if the plasma/CSF pathologic TDP-43 levels increase as the disease progresses. If this is true, plasma/CSF pathologic TDP-43 levels could be valuable for monitoring disease progression and evaluating treatment effectiveness.

If the plasma pathologic TDP-43 level reflects the severity of pathology in FTLD-TDP brains, plasma pathologic TDP-43 levels could further be used as a biomarker to monitor disease progression of FTLD-TDP and monitor the effectiveness of treatments for this disease. It is hypothesized that the worse neuronal loss and gliosis and/or TDP-43 pathology burden will be observed in the FTLD-TDP brain, and a higher the pathologic TDP-43 level will be observed in the plasma from patients with the most severe disease.

This study will include a subset of FTLD-TDP patients (n=30) that have both plasma/CSF and brain tissue (H&E slides and paraffin blocks of different brain regions) available. To semi quantify brain pathology, the frontal cortex and hippocampus of FTLD-TDP cases will be examined for neuronal loss and gliosis, as well as for TDP-43 burden. Neuronal loss and gliosis will be assessed on H&E-stained slides and will be graded as 0=none, 1=mild, 2=moderate, or 3=severe. An average score for neuronal loss/gliosis will be obtained from both brain regions. TDP-43 burden will be assessed on the frontal cortical and hippocampal sections immunostained with MAb No. 9. The overall TDP-43 burden will be graded on the same scale as that used for neuronal loss and gliosis, e.g., 0=none, 1=mild, 2=moderate, or 3=severe. Similarly, an average score for TDP-43 burden will be obtained from both brain regions. A linear model and generalized linear model, depending on the distribution of outcome, will be used with the plasma TDP-43 level as the covariate and the neuronal loss/gliosis or TDP-43 pathology burden as the outcome. Similar analyses will be performed using the CSF TDP-43 level as the covariate. The regression parameters using plasma or CSF TDP-43 levels will also be compared.

It is expected that the plasma pathologic TDP-43 level will reflect the pathology severity of FTLD-TDP brains. Plasma pathologic TDP-43 levels might show close correlation with either the severity of neuronal loss and gliosis or TDP-43 pathology burden, or both. The CSF pathologic TDP-43 level may show a closer correlation with brain pathology than the plasma pathologic TDP-43 level, which will be reassuring and will help validate plasma pathologic TDP-43 as a biomarker. There is a possibility that brain pathology and plasma pathologic TDP-43 levels show no obvious correlation. If this is the case, the FTLD-TDP brains will be divided into three groups based on their histopathologic type A, B, and C and brain pathology will be correlated with plasma pathologic TDP-43 levels separately for each group (FTLD-TDP type D is so rare, there are too few cases available for evaluation).

### A Novel MAb Against Human Pathologic TDP-43

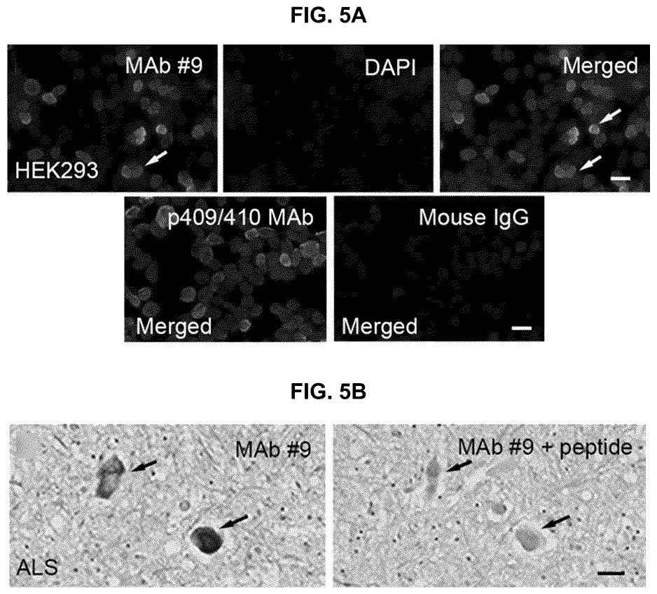

New TDP-43 MAbs were generated to develop a disease-specific biomarker of TDP-43 pathology. Murine MAbs were raised against human recombinant TDP-43. More than 5,000 MAb clones were screened by indirect enzyme-linked immunosorbent assay (ELISA). Promising MAbs were then tested by immunohistochemistry with brain samples from FTLD-TDP patients. Most of these antibodies showed robust immunoreactivity for normal nuclear TDP-43 and weak to strong reactivity for pathologic inclusions—except for MAb No. 9. MAb No. 9, whose epitope region is at the C-terminus, aa280-300, was strongly immunoreactive to all forms of pathologic TDP-43 inclusions, with minimal reactivity for normal TDP-43. The specificity of MAb No. 9 was further confirmed using HEK293 cells transfected with TDP-43-expressing plasmid and an absorption test (FIG. 5A-B).

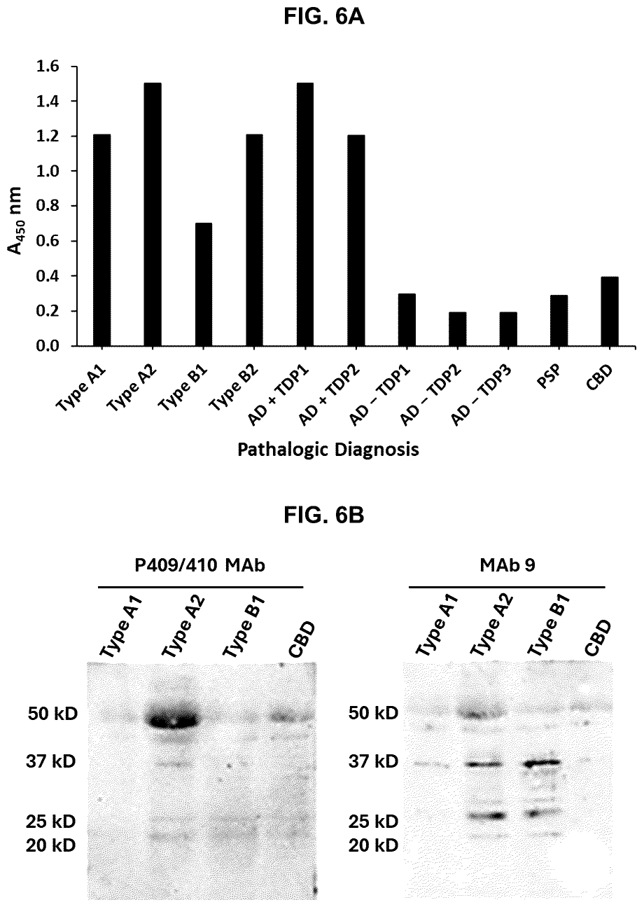

Interestingly, a sandwich ELISA using MAb No. 9 and another MAb (MAb No. 4) against the C-terminus of TDP-43 showed promise for detecting pathologic TDP-43 levels in the plasma (FIG. 6A) and CSF from patients of FTLD-TDP, and AD with TDP pathology. Thus, this ELISA system represents new and valuable tool for biomarker investigation and discovery and may prove useful in accelerating TDP-43 proteinopathy diagnoses and/or selecting TDP-43 patient for clinical trials of new therapeutic agents. Western blot with MAb No. 9 on some plasma samples (FTLD-type A1,-type A2,-type B1, and CBD (corticobasal degeneration) in FIG. 6A) revealed a different band pattern compared to that with the control antibody, p409/410 MAb (Cosmo Bio USA, Inc., Carlsbad, CA; FIG. 6B). Specifically, MAb No. 9 revealed stronger bands of 35 kDa than the p409/410 MAb, though both antibodies revealed the typical 45 and 25 kDa bands of pathologic TDP-43. It is worth noting that the 35 kDa TDP-43 fragment, though it has been reported in ALS, has rarely been seen in human tissue.

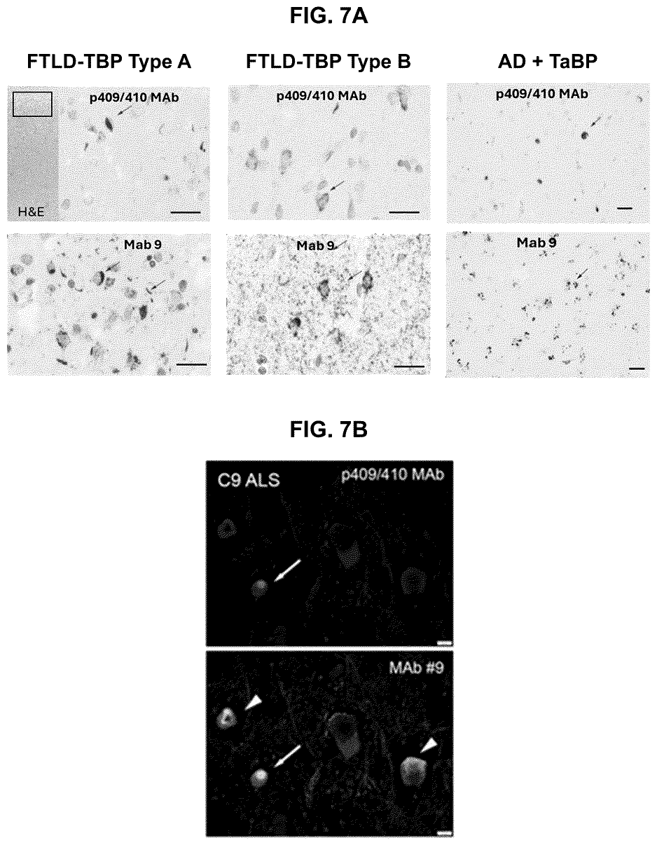

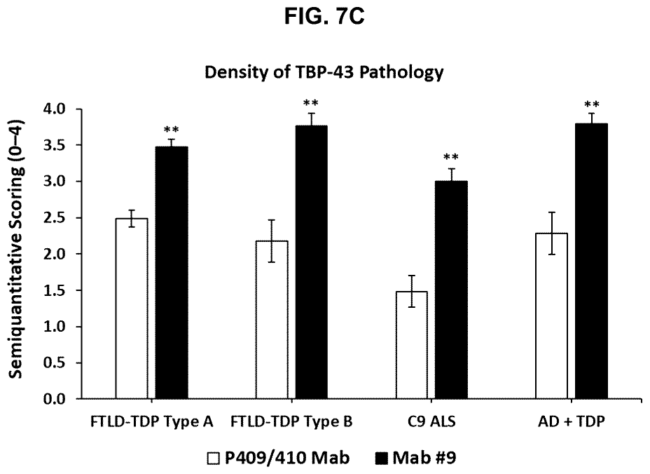

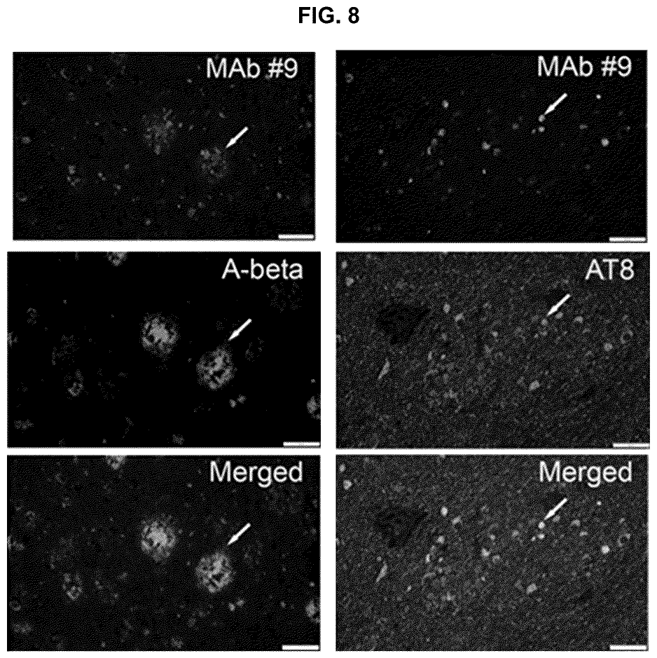

This ability of MAb No. 9 to preferentially detect pathologic TDP-43 inclusions is similar to that seen with phospho-TDP-43-specific MAbs, such as the p409/410 MAb, as evidenced by similar staining patterns (FIG. 7A-B). However, MAb No. 9 was more sensitive and could reveal an overall greater degree of TDP-43 pathology than the p409/410 MAb (FIG. 7C). Specifically, MAb No. 9 could reveal more fine neurites in the frontal cortex of FTLD-TDP type A brains and could reveal dense fine neurites in the frontal cortex of type B brains that are rarely revealed by the p409/410 MAb (FIG. 7A). In addition, the density of TDP-43-positive fine neurites revealed by MAb No. 9 was closely related to the severity of local neuronal loss (FIG. 7A), which was not reliably seen in studies using the p409/410 MAb. See e.g., Feneberg et al., Mol. Neurobiol. 55 (10): 7789-7801 (2018); Goossens et al., Acta Neuropathol. Commun. 3:15 (2015); Kawles et al., Brain 145 (3): 1069-1078 (2022). Interestingly, MAb No. 9 also revealed novel TDP-43 inclusions in cases of AD with medial temporal TDP-43 pathology (FIG. 7A and FIG. 8).

### Novel ELISA for Quantifying TDP-43

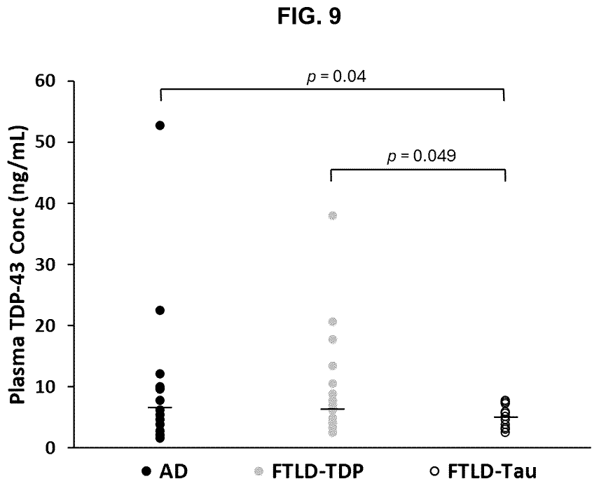

An ELISA was developed for quantifying TDP-43 levels by using the best antibody pair, MAb No. 9 and MAb No. 4. MAb No. 4 was used as the capture antibody, and MAb No. 9 was used as the detection antibody. After screening by checkerboard titration followed by ELISA, the optimal concentrations of capture MAb No. 4 was 0.5 μg/mL and that of the detection MAb No. 9 was 1:2000. Then the standard dose-response curve for TDP-43 was established by using the two-fold serially diluted recombinant rTDP-43 protein (OriGene Technologies, Rockville, MD). BSA was used to establish the baseline. Human plasma samples from 35 FTLD-TDP, 34 FTLD-Tau, and 22 AD patients were used for quantifying TDP-43 levels using the established ELISA system (FIG. 9).

### Example 2

### Abbreviations

AD, Alzheimer's Disease
ADNC, Alzheimer's Disease neuropathologic change
DN, dystrophic neurites
ELISA, enzyme-linked immunosorbent assay
FTLD, frontotemporal lobar degeneration
FTLD-TDP, frontotemporal lobar degeneration with TDP-43 pathology
GCI, glial cell inclusions
LATE-NC, limbic-predominant age-related TDP-43 encephalopathy neuropathological change
MAb, monoclonal antibody
NCI, neuronal cytoplasmic inclusions
NII, neuronal intranuclear inclusions
p409/410, the phosphorylated serine at amino acid 409/410 of the C-terminus of TDP-43
PAb, polyclonal antibody
TDP-43, transactive response DNA/RNA-binding protein of 43 kDa
ThD, combined threads and dot-like profiles

### Human Post-Mortem Cases

This project utilized brain samples from the Gift to Life Brain Bank at the University of Utah's Department of Pathology. A total of 63 paraformaldehyde-fixed, paraffin-embedded human brain samples were obtained for analysis. Demographic and neuropathological data for these cases are summarized in Table 2. The brain bank's samples have an average postmortem interval of 13.5 hours. For this study, brains with postmortem intervals exceeding 24 hours were excluded, as well as those from patients with significant medical conditions that could complicate pathological diagnosis (e.g., brain tumors).

The cohort represents a broad spectrum of clinical and genetic forms of primary TDP-43 proteinopathies (Table 2). It includes 30 FTLD-TDP cases spanning subtypes A, B, and C—specifically, 10 FTLD-TDP Type A cases (including five with GRN mutations), 10 FTLD-TDP Type B cases (including five with C9orf72 repeat expansions), and five FTLD-TDP Type C cases. Additionally, there are 10 ALS-TDP cases, five of which carry a pathogenic C9orf72 repeat expansion. The cohort also includes 10 cases with AD neuropathologic change (ADNC) alone, 10 ADNC cases with LATE-NC (comprising five cases each of LATE-NC stages 1, 2, and 3), and three healthy control (CON) cases. Pathologic characterization was conducted by board-certified neuropathologists following consensus criteria. Informed consent was obtained for all studies.

TABLE 2
Sample Demographics

| | | | Age at | Dis. | | | ADNC | LATE- |
|---|---|---|---|---|---|---|---|---|
| | Neuropathol. | Gender | Death | Durat. | | Clin. | Score | NC |
| Case | Diagnosis | (M/F) | (years) | (years) | Mutat. | Diagn. | (A, B, C) | stage |
| 1 | FTLD-TDP | M | 80 | 11 | None | FTD | 1, 1, 1 | NA |
| | Type A | | | | | | | |
| 2 | FTLD-TDP | F | 73 | 6 | None | PPA | 0, 1, 0 | NA |
| | Type A | | | | | | | |
| 3 | FTLD-TDP | F | 87 | 14 | None | PPA | 1, 0, 0 | NA |
| | Type A | | | | | | | |
| 4 | FTLD-TDP | M | 69 | 10 | None | FTD | 1, 1, 1 | NA |
| | Type A | | | | | | | |
| 5 | FTLD-TDP | M | 71 | 9 | None | FTD | 0, 1, 0 | NA |
| | Type A | | | | | | | |
| 6 | FTLD-TDP | M | 63 | 6 | GRN | FTD | 0, 1, 0 | NA |
| | Type A | | | | | | | |
| 7 | FTLD-TDP | F | 62 | 7 | GRN | Probable | 1, 0, 0 | NA |
| | Type A | | | | | AD | | |
| 8 | FTLD-TDP | M | 70 | 4 | GRN | PPA | 1, 0, 0 | NA |
| | Type A | | | | | | | |
| 9 | FTLD-TDP | M | 71 | 7 | GRN | FTD | 1, 1, 1 | NA |
| | Type A | | | | | | | |
| 10 | FTLD-TDP | F | 56 | 6 | GRN | PPA | 0, 1, 0 | NA |
| | Type A | | | | | | | |
| 11 | FTLD-TDP | M | 66 | 7 | None | FTD | 1, 1, 1 | NA |
| | Type B | | | | | | | |
| 12 | FTLD-TDP | F | 77 | 3 | None | FTD + ALS | 0, 1, 0 | NA |
| | Type B | | | | | | | |
| 13 | FTLD-TDP | M | 60 | 2 | None | FTD + ALS | 1, 0, 0 | NA |
| | Type B | | | | | | | |
| 14 | FTLD-TDP | M | 71 | 4 | None | FTD + ALS | 1, 1, 1 | NA |
| | Type B | | | | | | | |
| 15 | FTLD-TDP | M | 61 | 6 | None | FTD | 0, 1, 0 | NA |
| | Type B | | | | | | | |
| 16 | FTLD-TDP | F | 59 | 6 | C9orf72 | FTD | 0, 1, 0 | NA |
| | Type B | | | | | | | |
| 17 | FTLD-TDP | M | 61 | 4 | C9orf72 | FTD + ALS | 1, 0, 0 | NA |
| | Type B | | | | | | | |
| 18 | FTLD-TDP | F | 59 | 9 | C9orf72 | FTD | 1, 0, 0 | NA |
| | Type B | | | | | | | |
| 19 | FTLD-TDP | M | 62 | 3 | C9orf72 | FTD + ALS | 1, 1, 1 | NA |
| | Type B | | | | | | | |
| 20 | FTLD-TDP | M | 65 | 4 | C9orf72 | FTD + ALS | 0, 1, 0 | NA |
| | Type B | | | | | | | |
| 21 | FTLD-TDP | F | 69 | 11 | None | FTD | 0, 1, 0 | NA |
| | Type C | | | | | | | |
| 22 | FTLD-TDP | F | 66 | 13 | None | PPA | 1, 0, 0 | NA |
| | Type C | | | | | | | |
| 23 | FTLD-TDP | M | 72 | 15 | None | PPA | 1, 1, 1 | NA |
| | Type C | | | | | | | |
| 24 | FTLD-TDP | M | 74 | 10 | None | FTD | 0, 1, 0 | NA |
| | Type C | | | | | | | |
| 25 | FTLD-TDP | F | 82 | 9 | None | PPA | 1, 0, 0 | NA |
| | Type C | | | | | | | |
| 26 | ALS-TDP | F | 54 | 2 | None | ALS | 1, 1, 1 | NA |
| 27 | ALS-TDP | M | 75 | 4 | None | ALS | 0, 1, 0 | NA |
| 28 | ALS-TDP | M | 80 | 5 | None | ALS | 1, 0, 0 | NA |
| 29 | ALS-TDP | F | 67 | 6 | None | ALS | 1, 1, 1 | NA |
| 30 | ALS-TDP | F | 61 | 3 | None | ALS | 0, 1, 0 | NA |
| 31 | ALS-TDP | M | 59 | 3 | C9orf72 | ALS | 0, 1, 0 | NA |
| 32 | ALS-TDP | F | 67 | 4 | C9orf72 | ALS | 1, 0, 0 | NA |
| 33 | ALS-TDP | F | 74 | 5 | C9orf72 | ALS | 1, 0, 0 | NA |
| 34 | ALS-TDP | M | 74 | 4 | C9orf72 | ALS | 1, 1, 1 | NA |
| 35 | ALS-TDP | M | 77 | 3 | C9orf72 | ALS | 0, 1, 0 | NA |
| 36 | ADNC | M | 77 | 11 | None | Probable | 3, 3, 3 | NA |
| | | | | | | AD | | |
| 37 | ADNC | F | 81 | 9 | None | Probable | 3, 3, 3 | NA |
| | | | | | | AD | | |
| 38 | ADNC | F | 75 | 10 | None | Probable | 3, 3, 3 | NA |
| | | | | | | AD | | |
| 39 | ADNC | M | 77 | 11 | None | Probable | 3, 3, 3 | NA |
| | | | | | | AD | | |
| 40 | ADNC | F | 85 | 12 | None | Probable | 3, 3, 3 | NA |
| | | | | | | AD | | |
| 41 | ADNC | F | 75 | 10 | None | Probable | 3, 3, 3 | NA |
| | | | | | | AD | | |
| 42 | ADNC | M | 77 | 11 | None | Probable | 3, 3, 3 | NA |
| | | | | | | AD | | |
| 43 | ADNC | F | 76 | 13 | None | Probable | 3, 3, 3 | NA |
| | | | | | | AD | | |
| 44 | ADNC | F | 86 | 10 | None | Probable | 3, 3, 3 | NA |
| | | | | | | AD | | |
| 45 | ADNC | M | 77 | 11 | None | Probable | 3, 3, 3 | NA |
| | | | | | | AD | | |
| 46 | ADNC with | M | 78 | 12 | None | Probable | 3, 3, 3 | 3 |
| | LATE-NC | | | | | AD | | |
| | and HS | | | | | | | |
| 47 | ADNC with | F | 73 | 11 | None | Probable | 3, 3, 3 | 3 |
| | LATE-NC | | | | | AD | | |
| | and HS | | | | | | | |
| 48 | ADNC with | F | 82 | 13 | None | Probable | 3, 3, 3 | 3 |
| | LATE-NC | | | | | AD | | |
| | and HS | | | | | | | |
| 49 | ADNC with | M | 79 | 10 | None | Probable | 3, 3, 3 | 3 |
| | LATE-NC | | | | | AD | | |
| | and HS | | | | | | | |
| 50 | ADNC with | F | 69 | 12 | None | Probable | 3, 3, 3 | 3 |
| | LATE-NC | | | | | AD | | |
| 51 | ADNC with | M | 78 | 12 | None | Probable | 3, 3, 3 | 2 |
| | LATE-NC | | | | | AD | | |
| | and HS | | | | | | | |
| 52 | ADNC with | F | 75 | 11 | None | Probable | 3, 3, 3 | 2 |
| | LATE-NC | | | | | AD | | |
| 53 | ADNC with | F | 82 | 10 | None | Probable | 3, 3, 3 | 2 |
| | LATE-NC | | | | | AD | | |
| 54 | ADNC with | M | 78 | 12 | None | Probable | 3, 3, 3 | 2 |
| | LATE-NC | | | | | AD | | |
| 55 | ADNC with | F | 67 | 11 | None | Probable | 3, 3, 3 | 2 |
| | LATE-NC | | | | | AD | | |
| 56 | ADNC with | F | 82 | 9 | None | Probable | 3, 3, 3 | 1 |
| | LATE-NC | | | | | AD | | |
| 57 | ADNC with | M | 78 | 12 | None | Probable | 3, 3, 3 | 1 |
| | LATE-NC | | | | | AD | | |
| 58 | ADNC with | M | 73 | 10 | None | Probable | 3, 3, 3 | 1 |
| | LATE-NC | | | | | AD | | |
| 59 | ADNC with | F | 84 | 13 | None | Probable | 3, 3, 3 | 1 |
| | LATE-NC | | | | | AD | | |
| 60 | ADNC with | M | 76 | 9 | None | Probable | 3, 3, 3 | 1 |
| | LATE-NC | | | | | AD | | |
| 61 | CON | M | 72 | NA | None | NA | 0, 1, 0 | NA |
| 62 | CON | F | 69 | NA | None | NA | 1, 1, 0 | NA |
| 63 | CON | F | 79 | NA | None | NA | 0, 1, 0 | NA |

CON, healthy control; AD, Alzheimer's disease; ALS, amyotrophic lateral sclerosis; LATE-NC, limbic-predominant age-related TDP-43 encephalopathy neuropathological change; FTLD-TDP Type A, frontotemporal lobar degeneration with TDP-43 pathology Type A; ADNC, Alzheimer's disease neuropathologic change; FTD, frontotemporal dementia; HS, hippocampal sclerosis; NA, not applicable.

### Immunofluorescence and Immunohistochemistry

TDP-43 immunohistochemistry was performed as described in a previous study. Paraffin sections (5-μm) were deparaffinized, hydrated through xylenes and alcohol, and treated with 3% hydrogen peroxide in methanol for 10 minutes to block endogenous peroxidase activity. Antigen retrieval was carried out in a Decloaking Chamber™ with citrate buffer (pH 6.0) for 15 minutes. After washing, sections were blocked with 5% goat serum for 1 hour at room temperature. Sections were incubated with primary antibodies, including MAb No. 9 (1:4000) and a mouse monoclonal antibody targeting TDP-43 phosphorylated at serine 409/410 (pS409/410; 1:4000, v/v; Cosmo Bio Co., Ltd., Carlsbad, CA). Images were captured using an Olympus BX53 microscope with a DP74 camera, and brightness and contrast adjustments were made using cellSens Dimension software. Double-label immunofluorescence was performed using a rabbit polyclonal antibody against phosphorylated TDP-43 (pS409/410; 1:1000; Cosmo Bio Co., Ltd., Carlsbad, CA) and MAb No. 9 antiserum. Primary antibodies were incubated overnight at 4° C., followed by washing in Tris buffer and incubation with species-specific secondary antibodies (Alexa Fluor 488 anti-rabbit and Cy3 anti-mouse, 1:500) for 2 hours at room temperature. Sections were mounted in DAPI-containing Vectashield medium. Images were acquired using an Olympus BX53 microscope with a DP74 camera and cellSens Dimension software, in red and green channels. Colocalization was analyzed with the JACOP plugin in ImageJ. Pearson's correlation coefficients were calculated after threshold-based object recognition. Values ranged from 0 (no overlap) to 1 (complete colocalization), with values ≥0.5 considered significant.

### Semi-Quantitative Grading of Pathology

The anatomical regions analyzed in FTLD-TDP cases included the hippocampus, frontal cortex, dentate gyrus, and lower motor neuron regions, while those examined in ALS-TDP cases were the motor cortex and lower motor neurons. The inclusion types evaluated using p409/410 and MAb No. 9 immunohistochemistry were neuronal cytoplasmic inclusions (NCI), neuronal intranuclear inclusions (NII), dystrophic neurites (DN), combined threads and dot-like profiles (ThD), white matter threads, and glial cytoplasmic inclusions (GCI). For ADNC cases with or without LATE-NC, the analyzed regions included the frontal cortex, hippocampus, and amygdala. The inclusion types evaluated were the α-type (comprising NCI, NII, and DN) and β-type. Two investigators, blinded to diagnosis, assessed the extent of the TDP-43 pathology on a semi-quantitative scale (0=none, +=mild, ++=moderate, or +++=severe).

### Brain Extract Fractionation

Frozen frontal cortex samples (0.5 g) from FTLD-TDP Types A, B, and C, and control cases were subjected to sequential biochemical fractionation. Tissues were first homogenized in 10 volumes (5 mL) of buffer A (10 mM Tris-HCl, pH 7.5; 1 mM EGTA; 10% sucrose; 0.8 M NaCl). An additional 5 mL of buffer A containing 2% Triton X-100 was then added, and the homogenate was incubated at 37° C. for 30 minutes, followed by ultracentrifugation at 100,000×g for 30 minutes at 4° C. The resulting pellet was rehomogenized in 5 volumes of buffer A and incubated with 1% sarkosyl at 37° C. for 30 minutes. This mixture was again centrifuged at 100,000×g for 30 minutes at room temperature. The sarkosyl-insoluble pellet was then homogenized in 4 volumes of buffer A containing 1% CHAPS and centrifuged at 100,000×g for 20 minutes at room temperature. Finally, the pellet was sonicated in 0.6 volumes of 7 M guanidine hydrochloride and dialyzed overnight at 4° C. against 30 mM Tris-HCl (pH 7.5).

### Western Blot Analysis

Protein samples were resolved by 10% or 12% SDS-PAGE and transferred onto methanol-pretreated polyvinylidene difluoride (PVDF) membranes (Millipore, Temecula, CA). Membranes were incubated overnight at 4° C. with MAb No. 9 (1:4000 dilution). Following washes, immunoreactivity was detected using horseradish peroxidase (HRP)-conjugated goat anti-mouse lgG (1:10,000 dilution; Thermo Fisher Scientific, Rockford, IL), and visualized using enhanced chemiluminescence (ECL) reagents according to the manufacturer's protocol.

### Absorption Tests

To further validate antibody specificity, MAb No. 9 was preabsorbed with a synthetic peptide corresponding to amino acids 311-360 of TDP-43 (1 μg/mL; Thermo Fisher Scientific, Rockford, IL) prior to its application in Western blot analysis.

### Statistical Analysis

Statistical analysis was performed with GraphPad Prism software (version 10.2.2 for Windows). Wilcoxon tests were used to assess differences in pathology grades between MAb No. 9 and p409/410 immunohistochemistry. The significance level was set at p<0.05.

### Results

### MAb No. 9 Immunoreactivity in FTLD-TDP

To investigate potential differences in MAb No. 9 immunoreactivity across the spectrum of TDP-43 pathology, a cohort of 25 FTLD-TDP cases was analyzed—including subtypes A, B, and C, as well as cases with GRN and C9orf72 mutations—and 10 ALS-TDP cases (Table 2). A semi-quantitative assessment of the different types of MAb No. 9-positive inclusions was conducted in selected brain regions and these findings were compared to the amount of TDP-43 pathology detected using the p409/410 antibody, which serves as the gold standard.

### FTLD-TDP Type A

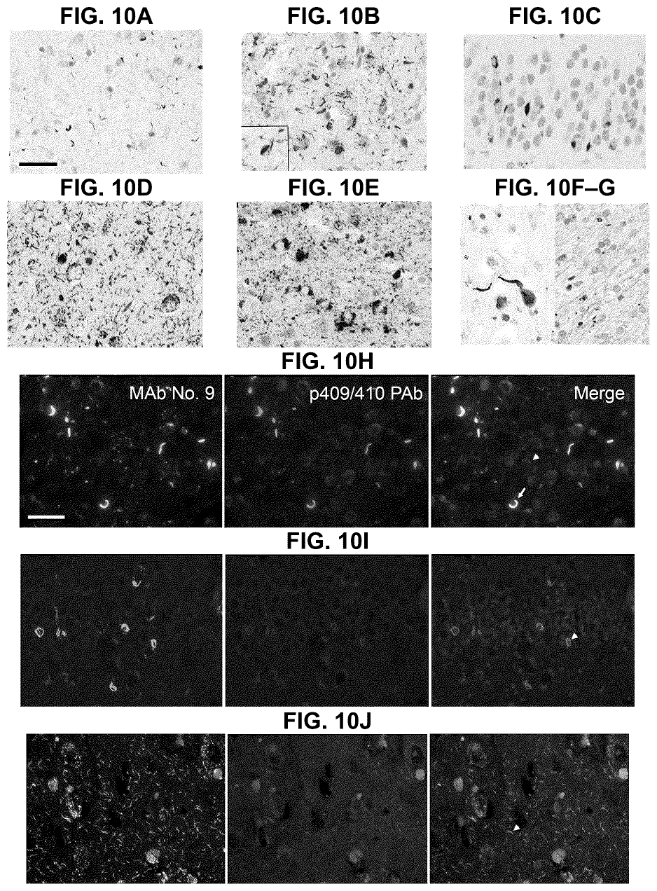

The cohort of FTLD-TDP Type A included 10 cases, five of which had a GRN mutation. All cases exhibited moderate-to-severe TDP-43 pathology, as detected by MAb No. 9 immunostaining in both cortical and subcortical regions. This pathology included small, compact NCI, DN, and NII in the frontotemporal gray matter and caudate, thread-like pathology in the white matter, and diffuse and compact NCI in the dentate gyrus, as well as threads in the CA1 region of the hippocampus. MAb No. 9 demonstrated a similar capacity to detect pathologic TDP-43 inclusions as the p409/410 MAb or polyclonal antibody (PAb), showing only minimal reactivity for normal nuclear TDP-43. However, MAb No. 9 revealed a higher overall level of TDP-43 pathology compared to the p409/410 MAb or PAb (FIG. 10). Notably, the increase in DN was more prominent than NCI and NII, such as in the frontal cortex of FTLD-TDP Type A brains (FIG. 10 Table 3).

TABLE 3
Semiquantitative Assessment of MAb No. 9-
immunopositive Pathology in FTLD-TDP Cases

| | | |
|---|---|---|
| MAb No. 9 | p409/410 | P value |

FTLD-TDP Type A

| | | | |
|---|---|---|---|
| Frontal Cortex | | | |
| (n = 10) | | | |
| Total | 2.7 ± 0.5 | 1.6 ± 0.5 | <0.01 |
| NCI | 1.8 ± 0.4 | 1.3 ± 0.5 | <0.05 |
| NII | 1.0 ± 0.0 | 1.0 ± 0.0 | ns |
| DN | 2.7 ± 0.5 | 1.6 ± 0.5 | <0.01 |
| WM threads | 1.8 ± 0.4 | 1.3 ± 0.5 | <0.05 |
| Dentate Gyrus | | | |
| (n=10) | | | |
| NCI | 1.8 ± 0.4 | 1.3 ± 0.5 | <0.05 |

FTLD-TDP Type B

| | | | |
|---|---|---|---|
| Frontal Cortex | | | |
| (n = 10) | | | |
| Total | 2.3 ± 0.7 | 1.6 ± 0.5 | <0.05 |
| NCI | 1.8 ± 0.4 | 1.3 ± 0.5 | <0.05 |
| NII | 0.0 ± 0.0 | 0.0 ± 0.0 | ns |
| DN | 2.6 ± 0.5 | 1.6 ± 0.5 | <0.01 |
| GCI | 1.5 ± 0.5 | 1.3 ± 0.5 | ns |
| Dentate Gyrus | | | |
| (n=10) | | | |
| NCI | 1.7 ± 0.5 | 1.2 ± 0.4 | <0.05 |
| Spinal Cord/Medulla | | | |
| (n = 10) | | | |
| Total | 1.7 ± 0.5 | 1.2 ± 0.5 | <0.05 |
| NCI | 1.6 ± 0.5 | 1.1 ± 0.3 | <0.05 |
| DN | 1.8 ± 0.8 | 1.1 ± 0.3 | <0.05 |
| GCI | 1.3 ± 0.5 | 1.2 ± 0.4 | ns |

ALS-TDP

| | | | |
|---|---|---|---|
| Motor Cortex | | | |
| (n = 10) | | | |
| Total | 1.6 ± 0.5 | 1.1 ± 0.3 | <0.05 |
| NCI | 1.6 ± 0.5 | 1.1 ± 0.3 | <0.05 |
| DN | 1.9 ± 0.3 | 1.3 ± 0.5 | <0.01 |
| GCI | 1.4 ± 0.5 | 1.2 ± 0.4 | ns |
| Spinal Cord/Medulla | | | |
| (n = 10) | | | |
| Total | 1.7 ± 0.5 | 1.2 ± 0.5 | <0.05 |
| NCI | 1.6 ± 0.5 | 1.1 ± 0.3 | <0.05 |
| DN | 1.9 ± 0.7 | 1.2 ± 0.4 | <0.05 |
| GCI | 1.4 ± 0.5 | 1.2 ± 0.4 | ns |

FTLD-TDP Type C

| | | | |
|---|---|---|---|
| Frontal Cortex | | | |
| (n = 5) | | | |
| Total | 1.6 ± 0.5 | 1.6 ± 0.5 | ns |
| NCI | 1.2 ± 0.4 | 1.2 ± 0.4 | ns |
| DN | 1.6 ± 0.5 | 1.6 ± 0.5 | ns |
| Dentate Gyrus | | | |
| (n = 5) | | | |
| NCI | 1.3 ± 0.5 | 1.3 ± 0.5 | ns |

NCI, neuronal cytoplasmic inclusions;
DN, dystrophic neuritis;
GCI, glial cytoplasmic inclusions;
NII, neuronal intranuclear inclusions;
WM, white matter;
ns, no significant difference;
Mean scores ± Standard Deviation

### FTLD-TDP Type B

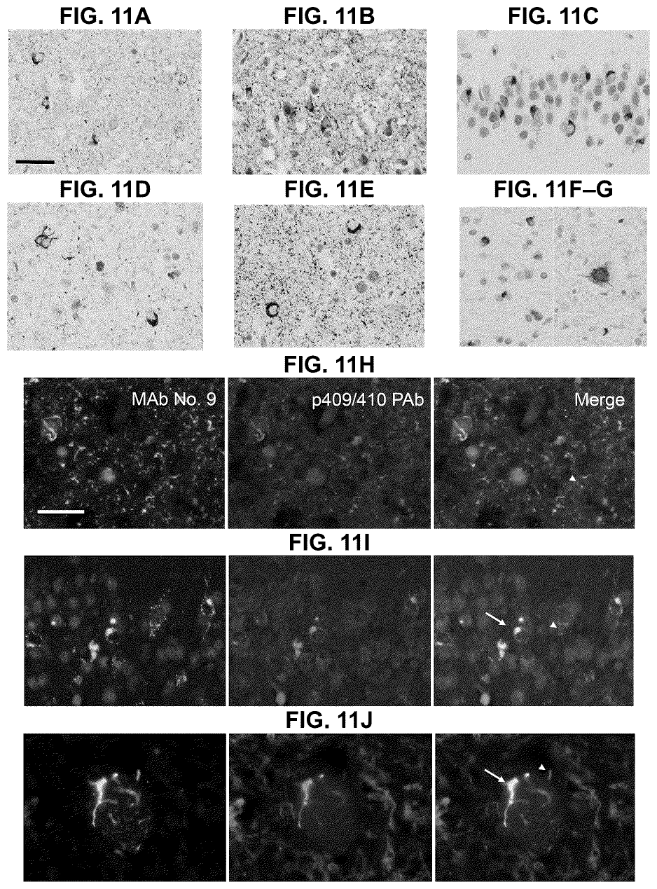

MAb No. 9 immunohistochemistry revealed strong and consistent labeling of neuronal and glial inclusions in all 10 cases with FTLD-TDP Type B pathology, including five with a C9orf72 repeat expansion (FIG. 11). Specifically, cases showed MAb No. 9-immunopositive diffuse and compact NCI, as well as DN, ThD, and GCI in affected cortical and subcortical regions, such as the frontal cortex, hippocampus, motor cortex, striatum, lower motor neurons, and white matter. MAb No. 9 revealed an overall greater degree of TDP-43 pathology than the p409/410 MAb or PAb (FIG. 11). Notably, the increase in ThD and DN was more prominent than the increases in 10 NCI and GCI, such as in the frontal cortex of FTLD-TDP Type B brains (FIG. 11, Table 3). Interestingly, MAb No. 9 revealed more preserved normal nuclear TDP-43 in FTLD-TDP Type B cases compared to Type A cases.

### ALS-TDP

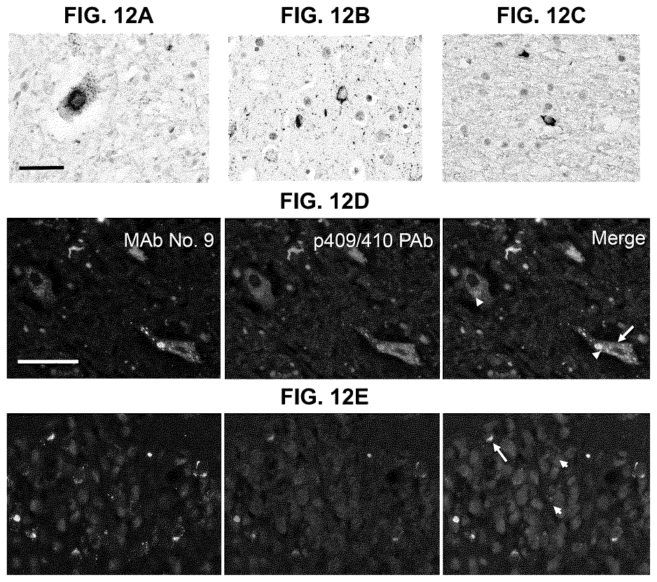

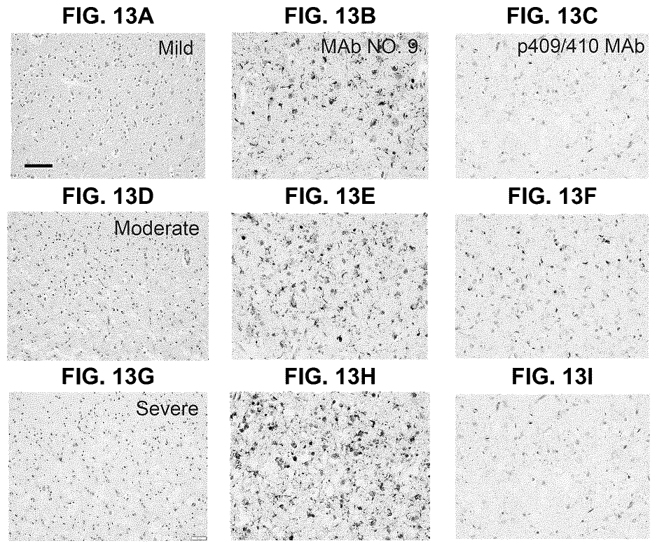

Strong immunoreactivity with MAb No. 9 was consistently observed across all hallmark TDP-43 inclusion types in the ALS-TDP cases analyzed (n=10), including those harboring C9orf72 mutations. Specifically, MAb No. 9 robustly labeled both diffuse and compact NCI, as well as DN, ThD, and GCI within the spinal cord and affected regions such as the precentral gyrus, and hippocampus (FIG. 12). Double-label immunofluorescence confirmed strong co-localization between MAb No. 9 and the 409/410 pAb across all inclusion types, regardless of their morphology (FIG. 13). Furthermore, MAb No. 9 revealed an overall greater degree of TDP-43 pathology than the p409/410 PAb in the spinal cord, precentral gyrus, and dentate gyrus (FIG. 12 and Table 3). Notably, the increase in ThD and DN was more prominent than the increases in NCI and GCI (FIG. 12, Table 3).

### MAb No. 9-Positive Pathology Remains Prominent in Severely Atrophic Brain Regions in FTLD-TDP Types A and B, and ALS-TDP.

Previous studies have shown that the density of TDP-43-positive inclusions detected using the phospho-specific p409/410 antibody often does not correlate—or may even inversely correlate—with local neurodegeneration 24,52,53. In advanced disease stages, these inclusions tend to diminish as affected neurons degenerate. In contrast, the current findings demonstrate that TDP-43 immunoreactivity detected by MAb No. 9 persists in regions with marked neuronal loss in FTLD-TDP Types A and B, and ALS-TDP, including the hippocampus, frontal and temporal cortices, and caudate nucleus (FIG. 13).

### FTLD-TDP Type C

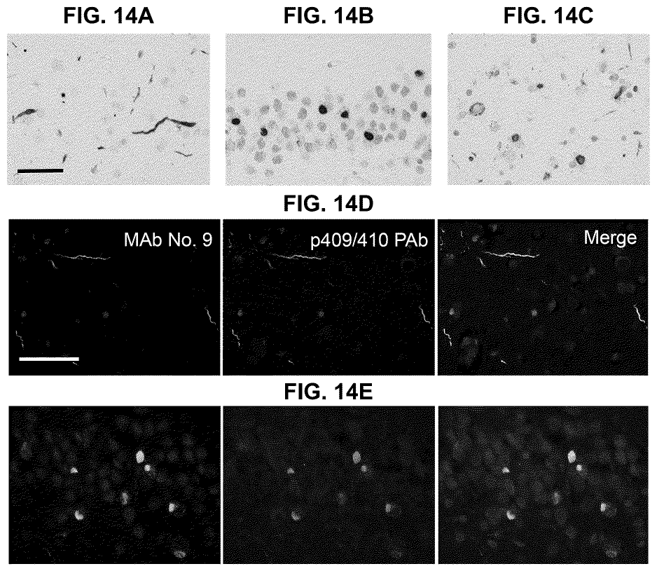

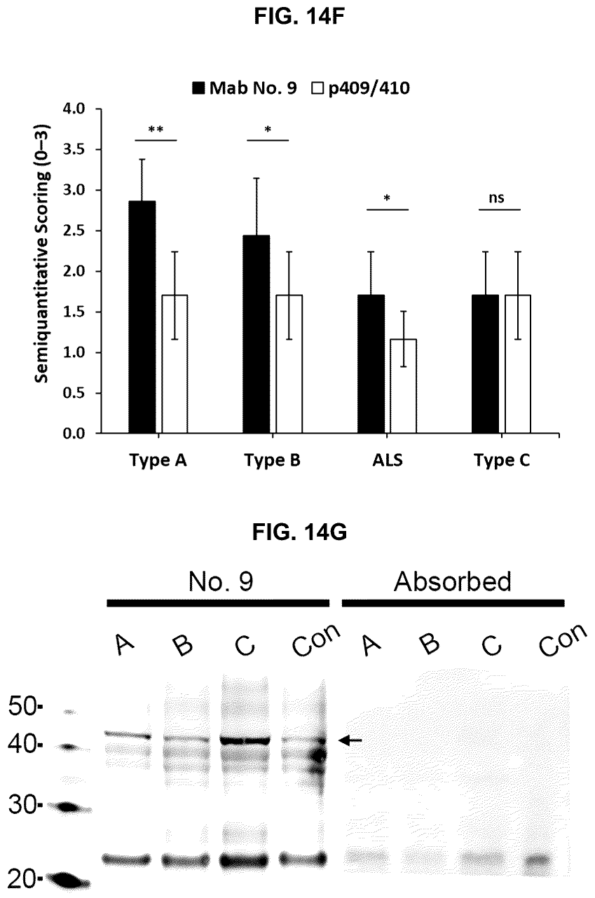

Strong immunoreactivity with MAb No. 9 was evident in characteristic Type C inclusions, specifically in the long DN within the neocortex; the round, compact Pick-like NCI in the dentate gyrus; and the NCI and DN in the striatum across all five FTLD-TDP Type C cases analyzed (FIG. 14). The level of TDP-43 pathology was comparable to that observed with p409/410 immunostaining. Double-label immunofluorescence confirmed complete colocalization between p409/410 PAb and MAb No. 9 in all inclusions (FIG. 14), with no score differences found in the semiquantitative evaluation of TDP-43 inclusions in the frontal cortex and hippocampus (Table 3). The subtype-specific increase in MAb No. 9-positive pathology is demonstrated by the semiquantitative densities of TDP-43 pathology in the frontal cortices of FTLD-TDP Type A, Type B, and Type C brains, as well as in the motor cortex of ALS-TDP brains (FIG. 14F).

Building on earlier observations, MAb No. 9 primarily recognizes full-length TDP-43 on Western blot. To complement the immunohistochemical findings and determine whether different FTLD-TDP subtypes predominantly exhibit the full-length form, immunoblot analyses was conducted using protein lysates extracted from frozen frontal cortical tissues of FTLD-TDP Types A, B, and C, as well as neurologically normal control brains (FIG. 5G). MAb No. 9 consistently detected a 43-kDa band corresponding to full-length TDP-43 across all FTLD-TDP subtypes, while only a faint physiological band was observed in the control sample. These results, together with the immunostaining data, support the interpretation that conformational changes in FTLD-TDP Types A and B may expose the MAb No. 9 epitope, rendering it accessible to standard immunostaining techniques.

MAb No. 9 Immunoreactivity in ADNC with LATE-NC

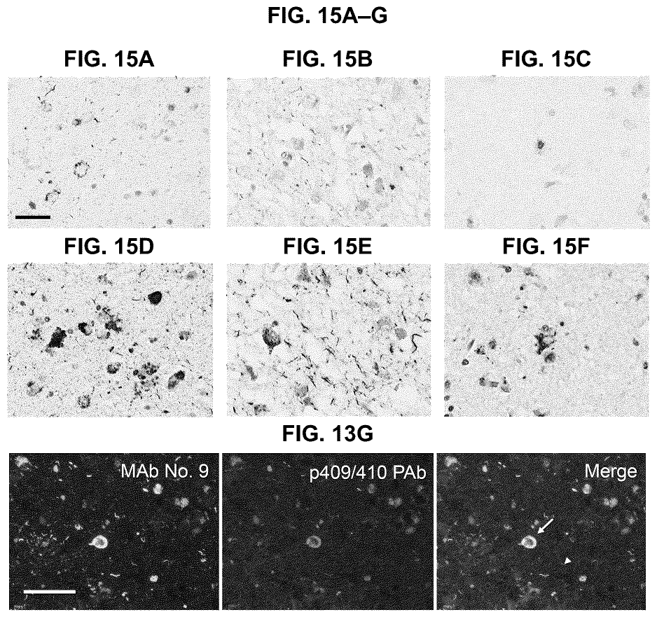

The immunoreactivity of MAb No. 9 was assessed in the amygdala, hippocampus, and frontal cortex of 15 ADNC brains with LATE-NC (five cases each in LATE-NC stages 1, 2, and 3) and 10 ADNC brains without LATE-NC. Immunohistochemistry for MAb No. 9 revealed labeling of TDP-43 Type α and Type β inclusions in all 15 cases of ADNC with LATE-NC (FIG. 15), while being immunonegative in the 10 cases of ADNC without LATE-NC. In ADNC with LATE-NC stage 1 and 2 cases, there was no difference in the density of MAb No. 9 and p409/410 immunopositivity in the amygdala and/or hippocampus (data not shown). MAb No. 9 revealed an overall greater degree of TDP-43 pathology, including of both Type α and Type β, than the p409/410 MAb or PAb in the amygdala and hippocampi of stage 3 LATE-NC cases. Although an increase in MAb No. 9 positivity was observed, the difference between MAb No. 9- and p409/410-positive pathology in the frontal cortex did not reach statistical significance (FIG. 15, Table 4).

TABLE 4
Semiquantitative Assessment of MAb No. 9-immunopositive
Pathology in Alzheimer's Disease neuropathologic
Change with Stage 3 LATE-NC

| | MAb No. 9 | p409/410 | P value |
|---|---|---|---|
| Amygdala | | | |
| (n = 5) | | | |
| Total | 2.8 ± 0.4 | 1.8 ± 0.8 | <0.05 |
| α | — | — | — |
| NCI | 2.6 ± 0.5 | 1.4 ± 0.5 | <0.01 |
| DN | 2.8 ± 0.4 | 1.8 ± 0.4 | <0.01 |
| β | 2.2 ± 0.4 | 1.4 ± 0.5 | <0.05 |
| Hippocampus | | | |
| (n = 5) | | | |
| Total | 2.8 ± 0.4 | 1.8 ± 0.8 | <0.05 |
| α | — | — | — |
| NCI | 2.6 ± 0.5 | 1.2 ± 0.4 | <0.01 |
| DN | 2.6 ± 0.5 | 1.8 ± 0.4 | <0.01 |
| β | 2.0 ± 0.0 | 1.4 ± 0.5 | <0.05 |
| Frontal Cortex | | | |
| (n = 5) | | | |
| Total | 1.4 ± 0.5 | 1.2 ± 0.4 | ns |
| α | — | — | — |
| NCI | 1.2 ± 0.4 | 1.0 ± 0.0 | ns |
| DN | 1.2 ± 0.4 | 1.0 ± 0.0 | ns |
| β | 1.1 ± 0.2 | 1.0 ± 0.0 | ns |

NCI, neuronal cytoplasmic inclusions;
DN, dystrophic neuritis;
ns, no significant difference;
Mean scores ± Standard Deviation

In this study, 60 cases of FTLD-TDP, ALS-TDP, and ADNC were analyzed with or without LATE-NC using MAb No. 9, whose specificity was previously validated through ELISA, Western blot, immunostaining, and absorption tests. Compared to the standard p409/410 antibody, MAb No. 9 showed increased reactivity in FTLD-TDP Types A and B, ALS-TDP (including C9orf72 and GRN mutation carriers), and ADNC with LATE-NC. FTLD-TDP Type C exhibited similar labeling patterns with both antibodies. ADNC cases without LATE-NC showed no immunoreactivity with either antibody.

Phosphorylation-specific antibodies, particularly pS409/410, remain among the most sensitive and specific markers for detecting the full spectrum of TDP-43 pathology. However, MAb No. 9 revealed distinct subtype-specific increases in immunoreactivity across FTLD-TDP and ALS-TDP. In Type A, including GRN mutation carriers, MAb No. 9 robustly labeled NCI, DN, NII, and white matter threads with greater intensity than p409/410. In Type B and ALS-TDP, including C9orf72 mutation carriers, it consistently detected diffuse and compact NCI, short DN, ThD, and GCI. The most pronounced increases were observed in DN and ThD across these subtypes. Notably, MAb No. 9 immunoreactivity persisted in regions with marked neuronal loss, especially in advanced disease stages, unlike p409/410, which often showed poor correlation with neurodegeneration. In contrast, FTLD-TDP Type C showed comparable labeling patterns between MAb No. 9 and p409/410, with both antibodies detecting NCI and long DN to a similar extent.

The enhanced detection by MAb No. 9 in FTLD-TDP Types A and B and ALS-TDP likely reflects its higher affinity; however, the pronounced labeling of DN and ThD-particularly in advanced disease stages-suggests that additional factors contribute to its selective recognition. The absence of enhanced labeling in Type C is unlikely to be due to tissue quality, as fixation parameters were consistent across all subtypes. Instead, these findings indicate that TDP-43 inclusions in Types A and B and ALS-TDP may contain structurally distinct species that are selectively recognized by MAb No. 9. Supporting this, recent cryo-EM studies have shown that aggregates in Types A and B form amyloid-like homodimeric fibrils, whereas Type C aggregates consist of heterodimers formed by TDP-43 and annexin A11 (ANXA 11). This structural divergence likely underlies the observed differences in immunoreactivity. Homodimer formation in Types A and B may induce a conformational change in the TDP-43 C-terminus, exposing the MAb No. 9 epitope to immunohistochemical detection. Consistent with this, Western blot analysis demonstrated that MAb No. 9 predominantly recognizes full-length TDP-43 across all subtypes, indicating that the epitope remains intact under denaturing conditions, although its accessibility in situ likely varies depending on aggregate conformation. These findings underscore the importance of conformational context in epitope exposure and suggest that MAb No. 9 detects TDP-43 in a conformation-dependent and subtype-specific manner.

In ADNC cases with LATE-NC, MAb No. 9 robustly labeled all TDP-43-positive inclusions, including both α and β types. It also revealed more extensive pathology compared to the p409/410 antibody; however, this increased detection was limited to the amygdala and hippocampus in stage 3 LATE-NC cases. In stages 1 and 2, the density of TDP-43 pathology detected by MAb No. 9 was comparable to that revealed by p409/410. Thus, the staging of LATE-NC based on MAb No. 9 immunoreactivity aligns with that determined by p409/410. The increased MAb No. 9-positive pathology in ADNC with LATE-NC, resembling that observed in FTLD-TDP Types A and B, suggests that both conditions may share similar MAb No. 9-reactive TDP-43 species. However, notable differences also exist. In ADNC with LATE-NC, the extent of MAb No. 9-positive pathology is dependent on both the LATE-NC stage and the affected brain region. Moreover, there is a substantial difference in the overall distribution and severity of pathology between FTLD-TDP and LATE-NC, as reported by multiple studies. Additional distinctions include differences in age of onset (with FTLD typically affecting younger individuals), clinical presentation, and genetic risk factors. These findings suggest that while ADNC with LATE-NC may harbor TDP-43 species similar to those in FTLD-TDP, the underlying mechanisms and disease processes are likely distinct.

This study focused on the most common primary TDP-43 proteinopathies, but rarer FTLD-TDP subtypes—such as Type D (associated with VCP mutations), Type E, and genetically linked cases involving TARDBP, UBQLN2, OPTN, and TBK1 mutations—also exist. Future research will be essential to determine how these subtypes are classified based on MAb No. 9 immunoreactivity.

Another promising direction for future investigation is the role of the TDP-43 epitope recognized by MAb No. 9 in disease pathogenesis. Determining whether this epitope contributes to aggregation, propagation, or toxicity could offer critical insights into the mechanisms driving neurodegeneration. Given the urgent need for FTLD-TDP-specific biomarkers, developing ELISAs to measure TDP-43 levels in cerebrospinal fluid and plasma is vital—not only for improving diagnostic accuracy but also for monitoring responses to disease-modifying therapies in FTLD-TDP and related conditions. Additionally, exploring the therapeutic potential of MAb No. 9 is crucial. Studies assessing its ability to modulate pathogenic TDP-43 species, reduce neuronal loss, or alter disease progression could pave the way for targeted immunotherapies in FTLD-TDP and ADNC with LATE-NC.

These findings demonstrate that MAb No. 9 selectively detects novel TDP-43 pathology in FTLD-TDP Types A and B, ALS-TDP, and ADNC with LATE-NC, but not in FTLD-TDP Type C-likely due to conformation-dependent differences. These results support the hypothesis that distinct TDP-43 conformers contribute to the phenotypic diversity of TDP-43 proteinopathies. The newly characterized non-phosphorylated (C-terminal) TDP-43 antibody, MAb No. 9, thus represents a valuable tool for advancing the understanding of the structural and biochemical mechanisms underlying FTLD-TDP and ADNC with LATE-NC.

### Example 3

### Development of a Sandwich ELISA System for Pathological TDP-43 Detection

Monoclonal antibodies were biotinylated and screened using a checkerboard ELISA to identify optimal antibody pairs for detecting pathological TDP-43. Based on specificity and sensitivity, the antibody pair consisting of MAb No. 9 (capture antibody) and MAb p409/410 (detection antibody) was selected. This pair, referred to as MAb No. 9-409/410, demonstrated superior performance in recognizing disease-associated TDP-43 species. Initially validated in a standard ELISA format, the MAb No. 9-409/410 pair was subsequently adapted to the Meso Scale Discovery (MSD) platform to enhance assay sensitivity and reduce sample volume requirements, enabling more efficient and scalable biomarker detection.

### Biotin Labeling of Anti-TDP-43 Antibodies

To prepare biotin-labeled antibodies for enhanced detection sensitivity, 1 mg of Sulfo-NHS-LC-Biotin (Thermo Fisher Scientific, Waltham, MA, USA) was first dissolved in 180 μL of deionized water. A volume of 250 μL of purified antibody, at a concentration of 2-2.5 mg/mL, was then mixed with 13.5 μL of the biotin solution. This mixture was incubated at 4° C. in the dark for 2 hours to allow for efficient conjugation. Following incubation, unbound biotin was removed using a desalting column (Thermo Fisher Scientific), and the labeled antibody was stabilized by adding an equal volume of glycerol. The final product was stored at −20° C. until use. To confirm successful biotinylation, a direct ELISA was performed. The labeled antibody was coated onto microtiter plates and detected using Avidin-HRP (Vector Laboratories, Newark, CA, USA) and TMB substrate (Bio-Rad Laboratories, Hercules, CA, USA). The reaction was quantified by measuring absorbance at 450 nm using a microplate reader.

### MSD Procedure

The capture antibody (Monoclonal antibody No. 9) was coated onto MSD Standard Plates (Catalog #L15XA-3, Meso Scale Discovery, Rockville, MD, USA) by adding 40 μL per well at a concentration of 0.5 μg/mL in PBS. Plates were sealed and incubated overnight at 4° C. The plate was washed once with 150 μL of PBS+0.05% Tween-20 (PBST), then tapped dry. A 150 μL aliquot of blocking buffer (5% Blocker A in PBST; Blocker A, Catalog #R93AA-2, MSD) was added to each well. Plates were sealed and incubated at ambient temperature for 1 hour with shaking at 700 RPM. After washing once with 150 μL PBST and tapping dry, 50 μL of sample or control plasma was added to each well. Plates were sealed and incubated at ambient temperature for 1 hour with shaking at 700 RPM. The wells were washed three times with 150 UL PBST and tapped dry. The detection antibody (biotin-labeled p409/410 monoclonal antibody) was diluted 1:1000 in assay diluent (1% Blocker A in PBS) and added at 50 μL per well. Plates were sealed and incubated at ambient temperature for 1 hour with shaking at 700 RPM. After three washes with 150 μL PBST, Sulfo-TAG labeled streptavidin (Catalog #R32AD-5, MSD), diluted 1:1000 in assay diluent, was added (50 μL per well). Plates were sealed and incubated at ambient temperature for 1 hour with shaking at 700 RPM. Following three final washes with 150 μL PBST, 150 μL of GOLD Read Buffer B (Catalog #R60AM-3, MSD) was added to each well. Plates were read using the MESO QuickPlex SQ 120 instrument (Meso Scale Discovery).

### Patient Blood Sample Collection and Grouping

Peripheral blood samples were collected from patients diagnosed with frontotemporal lobar degeneration with TDP-43 pathology (FTLD-TDP), FTLD-tau, and healthy aging controls. In a subset of participants (N=10 per group), plasma samples were analyzed before and after treatment with 2% SDS to assess the release of pathological TDP-43 (see below). An expanded cohort (N=20 per group) was used to compare post-treatment plasma TDP-43 levels across FTLD-TDP, LATE-NC, FTLD-tau, and healthy controls. Additionally, plasma extracellular vesicle (EV) TDP-43 levels were measured in FTLD-TDP, FTLD-tau, and healthy controls to evaluate the biomarker potential of EV-derived TDP-43.

### Extracellular Vesicle (EV) Isolation and Lysis

EVs were isolated from human plasma using established differential ultracentrifugation protocols. Briefly, plasma samples were subjected to sequential centrifugation steps to remove cells, debris, and larger vesicles, followed by ultracentrifugation at 100,000×g for 70 minutes to pellet EVs. The EV pellets were washed in PBS and re-pelleted to ensure purity.

To optimize the workflow for clinical application, multiple lysis buffers were evaluated for their efficiency in releasing TDP-43 from EVs. Tested buffers included Triton X-100 (1%), NP-40 (1%), RIPA, and SDS at varying concentrations (0.5-2%). Among these, 2% SDS consistently yielded the highest recovery of TDP-43, as determined by immunoblotting and ELISA using MAb No. 9. This buffer was selected for subsequent analyses due to its superior performance and compatibility with downstream detection assays.

All procedures were performed under standardized conditions to ensure reproducibility and minimize protein degradation. The optimized EV lysis protocol provides a streamlined and scalable approach suitable for clinical diagnostic workflows.

### Blood Sample Preparation

Patient plasma samples are treated with 2% SDS to release the proteins from EV and enhance antigen accessibility. Specifically, 50 μL of 2% SDS solution was added to 50 μL of plasma, followed by incubation at room temperature for 10 minutes.

### Statistical Analysis

Statistical comparisons were performed using paired and unpaired t-tests where appropriate. A p-value <0.01 was considered statistically significant.

### Results

### Antibody Screening and Pair Selection

Over 5,000 monoclonal antibody clones were screened using recombinant human TDP-43 expressed in E. coli. Among these, MAb No. 9 emerged as the top candidate due to its phosphorylation-independent binding and strong immunoreactivity to pathological TDP-43 inclusions, with minimal reactivity to normal nuclear TDP-43. Additional antibodies targeting the phosphorylated s409/410 epitope were generated and tested in various pairings (e.g., MAb Nos. 9+5; 9+14; 4+6), but the MAb No. 9-409/410 pair consistently showed the best performance in both ELISA and MSD formats.

### MSD Assay Performance Across Antibody Pairs

Multiple antibody pairs were evaluated using MSD assays on plasma samples from FTLD-TDP, FTLD-tau, and healthy controls. The 9-409/410 antibody pair produced stronger signals and more distinct group differentiation compared to other combinations (e.g., 9-5, 9-14), which showed variable performance and lower sensitivity and specificity.

### Freeze-Thaw Effects on Plasma TDP-43 Levels

A moderate increase in plasma TDP-43 levels was observed in FTLD-TDP patients following freeze-thaw cycles, suggesting that EVs may release TDP-43 upon disruption. This finding prompted further investigation into EV-derived TDP-43 as a biomarker.

### Plasma EV TDP-43 Detection

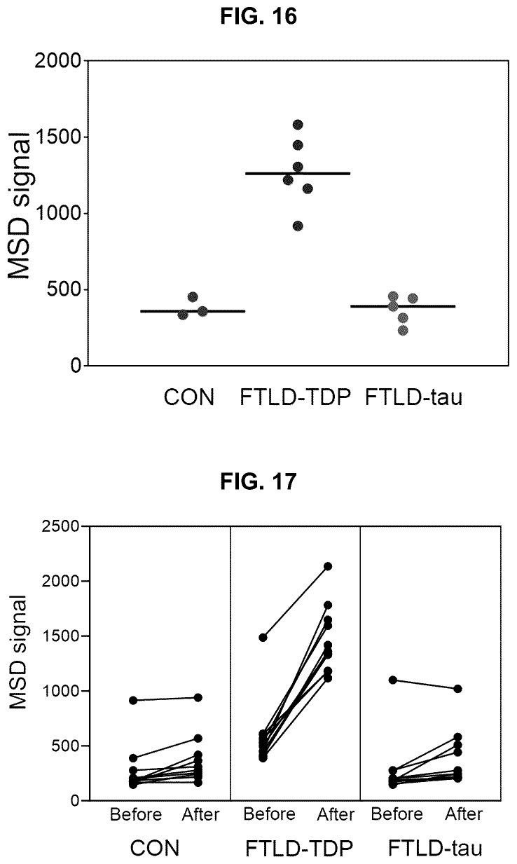

Using the 9-409/410 antibody pair, TDP-43 levels were measured in plasma EVs from FTLD-TDP, FTLD-tau, and healthy aging controls. FTLD-TDP patients exhibited significantly higher EV TDP-43 levels compared to other groups (FIG. 16), supporting the hypothesis that EVs reflect CNS pathology.

### EV Lysis Optimization

To overcome the technical challenges associated with EV isolation and protein extraction, a panel of detergents was systematically evaluated for their ability to lyse EVs and release TDP-43 while preserving antigenicity. Buffers tested included Triton X-100, NP-40, Tween 20, and SDS at concentrations ranging from 0.5% to 2%. Among these, SDS at 1-2% final concentration demonstrated the highest efficiency in releasing TDP-43, with minimal interference in antibody binding during immunodetection. The optimized 2% SDS protocol offers a simple, reproducible, and clinically adaptable method for EV lysis and biomarker quantification.

EV Lysis with 2% SDS Enhances Detection

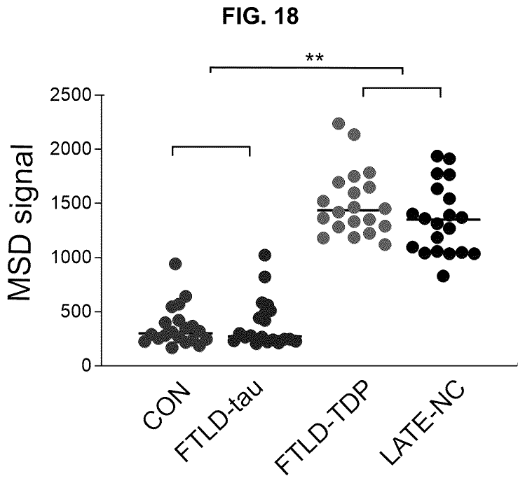

Treatment of plasma-derived EVs with 2% SDS significantly increased the release of TDP-43 in samples from FTLD-TDP patients (p<0.01, n=10), as measured by MAb No. 9-based immunoassays. In contrast, no significant increase in TDP-43 levels was observed in FTLD-tau or control samples following SDS treatment (FIG. 17). Post-lysis, plasma TDP-43 levels in FTLD-TDP and LATE-NC patients were markedly elevated compared to FTLD-tau and control groups (p<0.01, n=20; FIG. 18), supporting the specificity of the assay for TDP-43 proteinopathy.

### Advantages of the EV-Lysis Approach

This method eliminates the need for complex EV isolation, reduces sample processing time to under 15 minutes, and enables high-throughput screening. It offers a cost-effective and scalable platform for liquid biomarker development, with strong potential for clinical translation in early diagnosis and disease monitoring.

### DISCUSSION

This study presents a novel and clinically feasible approach for detecting pathological TDP-43 in plasma, offering significant implications for the diagnosis and monitoring of TDP-43 proteinopathies. Using the highly specific monoclonal antibody pair MAb No. 9-409/410, a Meso Scale Discovery (MSD) assay was successfully adapted to measure TDP-43 levels in plasma and plasma-derived extracellular vesicles (EVs). The results demonstrate that EV-lysed plasma TDP-43 levels can serve as a robust biomarker for FTLD-TDP and LATE-NC, distinguishing these conditions from FTLD-tau and healthy aging controls.

Initial MSD assays revealed low levels of TDP-43 in plasma across all groups, consistent with previous reports of limited sensitivity in detecting brain-derived pathological TDP-43 in peripheral blood. However, a key insight emerged when freeze-thaw cycles modestly increased TDP-43 levels in FTLD-TDP plasma samples, suggesting that EVs may harbor disease-relevant TDP-43 species. This led to a pilot study which confirmed elevated plasma EV TDP-43 levels in FTLD-TDP patients compared to FTLD-tau and controls.

Recognizing the technical challenges of EV isolation, a rapid and efficient lysis protocol was developed using 2% SDS. This method significantly enhanced TDP-43 detection in plasma and reduced sample processing time from hours to just 15 minutes. Post-treatment with 2% SDS, TDP-43 levels were significantly higher in FTLD-TDP and LATE-NC patients than in FTLD-tau and control groups, supporting the utility of EV-lysed plasma TDP-43 as a biomarker.

Importantly, the specificity of the 9-409/410 antibody pair enabled selective detection of pathological TDP-43, even when mixed with normal TDP-43 in plasma. This is critical for clinical application, as it allows for accurate differentiation between TDP-43-related and non-TDP-43-related neurodegenerative conditions. The observed elevation of TDP-43 in rare cases across all groups prior to treatment also highlights the need for further investigation into the role of free TDP-43 in plasma and its potential confounding factors.

Overall, these findings support the feasibility of a blood-based diagnostic test for TDP-43 proteinopathy. The EV-lysis approach offers a scalable, time-efficient, and clinically practical alternative to traditional EV isolation methods. Future studies should focus on validating this method in larger cohorts, exploring its correlation with disease severity and progression, and integrating it into multi-modal biomarker panels for neurodegenerative diseases.

This study may be limited by its modest sample size and cross-sectional design. Variability in EV content and potential confounding factors such as comorbidities or medication effects were not fully controlled. Future studies should validate these findings in larger, longitudinal cohorts and explore integration with other biomarkers for improved diagnostic accuracy.

### Sequences

| SEQ ID | | |
|---|---|---|
| NO | NAME | SEQUENCE (5′→3′ or N→C) |
| 1 | TDP-43 Full | atgtctgaatatattcgggtaaccgaagatgagaacgatgagcccattgaaata |
| | length NT | ccatcggaagacgatgggacggtgctgctctccacggttacagcccagtttcca |
| | | ggggcgtgtgggcttcgctacaggaatccagtgtctcagtgtatgagaggtgtc |
| | | cggctggtagaaggaattctgcatgccccagatgctggctggggaaatctggtg |
| | | tatgttgtcaactatccaaaagataacaaaagaaaaatggatgagacagatgct |
| | | tcatcagcagtgaaagtgaaaagagcagtccagaaaacatccgatttaatagtg |
| | | ttgggtctcccatggaaaacaaccgaacaggacctgaaagagtattttagtacc |
| | | tttggagaagttcttatggtgcaggtcaagaaagatcttaagactggtcattca |
| | | aaggggtttggctttgttcgttttacggaatatgaaacacaagtgaaagtaatg |
| | | tcacagcgacatatgatagatggacgatggtgtgactgcaaacttcctaattct |
| | | aagcaaagccaagatgagcctttgagaagcagaaaagtgtttgtggggcgctgt |
| | | acagaggacatgactgaggatgagctgcgggagttcttctctcagtacggggat |
| | | gtgatggatgtcttcatccccaagccattcagggcctttgcctttgttacattt |
| | | gcagatgatcagattgcgcagtctctttgtggagaggacttgatcattaaagga |
| | | atcagcgttcatatatccaatgccgaacctaagcacaatagcaatagacagtta |
| | | gaaagaagtggaagatttggtggtaatccaggtggctttgggaatcagggtgga |
| | | tttggtaatagcagagggggtggagctggtttgggaaacaatcaaggtagtaat |
| | | atgggtggtgggatgaactttggtgcgttcagcattaatccagccatgatggct |
| | | gccgcccaggcagcactacagagcagttggggtatgatgggcatgttagccagc |
| | | cagcagaaccagtcaggcccatcgggtaataaccaaaaccaaggcaacatgcag |
| | | agggagccaaaccaggccttcggttctggaaataactcttatagtggctctaat |
| | | tctggtgcagcaattggttggggatcagcatccaatgcagggtcgggcagtggt |
| | | tttaatggaggctttggctcaagcatggattctaagtcttctggctggggaatg |
| 2 | TDP-43 Full | MSEYIRVTEDENDEPIEIPSEDDGTVLLSTVTAQFPGACGLRYRNPVSQCMRGV |
| | length AA | RLVEGILHAPDAGWGNLVYVVNYPKDNKRKMDETDASSAVKVKRAVQKTSDLIV |
| | | LGLPWKTTEQDLKEYFSTFGEVLMVQVKKDLKTGHSKGFGFVRFTEYETQVKVM |
| | | SQRHMIDGRWCDCKLPNSKQSQDEPLRSRKVFVGRCTEDMTEDELREFFSQYGD |
| | | VMDVFIPKPFRAFAFVTFADDQIAQSLCGEDLIIKGISVHISNAEPKHNSNRQL |
| | | ERSGRFGGNPGGFGNQGGFGNSRGGGAGLGNNQGSNMGGGMNFGAFSINPAMMA |
| | | AAQAALQSSWGMMGMLASQQNQSGPSGNNQNQGNMQREPNQAFGSGNNSYSGSN |
| | | SGAAIGWGSASNAGSGSGFNGGFGSSMDSKSSGWGMNH |
| 3 | AA260-AA360 of | gccgaacctaagcacaatagcaatagacagttagaaagaagtggaagatttggt |
| | TDP-43 NT | ggtaatccaggtggctttgggaatcagggtggatttggtaatagcagagggggt |
| | | ggagctggtttgggaaacaatcaaggtagtaatatgggtggtgggatgaacttt |
| | | ggtgcgttcagcattaatccagccatgatggctgccgcccaggcagcactacag |
| | | agcagttggggtatgatgggcatgttagccagccagcagaaccagtcaggccca |
| | | tcgggtaataaccaaaaccaaggcaacatgcag |
| 4 | AA260-AA360 of | AEPKHNSNRQLERSGRFGGNPGGFGNQGGFGNSRGGGAGLGNNQGSNMGGGMNF |
| | TDP-43 AA | GAFSINPAMMAAAQAALQSSWGMMGMLASQQNQSGPSGNNQNQGNMQ |
| 5 | HC anti-TDP-43 | caggtgcagctgggcgaaagcggcccggaactgaaaaaaccgggcgaaaccgtg |
| | antibody MAb 9 | aaaattagctgcaaagcgagcggcgatacctttaccgcgaacaccatgcattgg |
| | (6B10) NT | gtgaaacagagcccgggccgcggctttaaaagcctggaatggattgataccagc |
| | | tatagcggcgaaacccgcgcgcatgattttggcggccgctttgcgtttagcctg |
| | | gaaaccagccagagcaccgcgtatctgcagatgcagaacctgaaaaccgaagat |
| | | accgcgatttatttttgcgtgcgcggcctggcggattattggggccagggcacc |
| | | aaagtgaccgtgagcagcagcgctaaaacgacacccccatctgtctatccactg |
| | | gcccctggatctgctgcccaaactaactccatggtgaccctgggatgcctggtc |
| | | aagggctatttccctgagccagtgacagtgacctggaactctggatccctgtcc |
| | | agcggtgtgcacaccttcccagctgtcctgcagtctgacctctacactctgagc |
| | | agctcagtgactgtcccctccagcacctggcccagcgagaccgtcacctgcaac |
| | | gttgcccacccggccagcagcaccaaggtggacaagaaaattgtgcccagggat |
| | | tgtggttgtaagccttgcatatgtacagtcccagaagtatcatctgtcttcatc |
| | | ttccccccaaagcccaaggatgtgctcaccattactctgactcctaaggtcacg |
| | | tgtgttgtggtagacatcagcaaggatgatcccgaggtccagttcagctggttt |
| | | gtagatgatgtggaggtgcacacagctcagacgcaaccccgggaggagcagttc |
| | | aacagcactttccgctcagtcagtgaacttcccatcatgcaccaggactggctc |
| | | aatggcaaggagttcaaatgcagggtcaacagtgcagctttccctgcccccatc |
| | | gagaaaaccatctccaaaaccaaaggcagaccgaaggctccacaggtgtacacc |
| | | attccacctcccaaggagcagatggccaaggataaagtcagtctgacctgcatg |
| | | ataacagacttcttccctgaagacattactgtggagtggcagtggaatgggcag |
| | | ccagcggagaactacaagaacactcagcccatcatggacacagatggctcttac |
| | | ttcgtctacagcaagctcaatgtgcagaagagcaactgggaggcaggaaatact |
| | | ttcacctgctctgtgttacatgagggcctgcacaaccaccatactgagaagagc |
| | | ctctcccactctcctggtaaa |
| 6 | HC anti-TDP-43 | QVQLGESGPELKKPGETVKISCKASGDTFTANTMHWVKQSPGRGFKSLEWIDTS |
| | antibody MAb 9 | YSGETRAHDFGGRFAFSLETSQSTAYLQMQNLKTEDTAIYFCVRGLADYWGQGT |
| | (6B10) AA | KVTVSSSAKTTPPSVYPLAPGSAAQTNSMVTLGCLVKGYFPEPVTVTWNSGSLS |
| | | SGVHTFPAVLQSDLYTLSSSVTVPSSTWPSETVTCNVAHPASSTKVDKKIVPRD |
| | | CGCKPCICTVPEVSSVFIFPPKPKDVLTITLTPKVTCVVVDISKDDPEVQFSWF |
| | | VDDVEVHTAQTQPREEQFNSTFRSVSELPIMHQDWLNGKEFKCRVNSAAFPAPI |
| | | EKTISKTKGRPKAPQVYTIPPPKEQMAKDKVSLTCMITDFFPEDITVEWQWNGQ |
| | | PAENYKNTQPIMDTDGSYFVYSKLNVOKSNWEAGNTFTCSVLHEGLHNHHTEKS |
| | | LSHSPGK |
| 7 | VH anti-TDP-43 | caggtgcagctgggcgaaagcggcccggaactgaaaaaaccgggcgaaaccgtg |
| | antibody MAb 9 | aaaattagctgcaaagcgagcggcgatacctttaccgcgaacaccatgcattgg |
| | (6B10) NT | gtgaaacagagcccgggccgcggctttaaaagcctggaatggattgataccagc |
| | | tatagcggcgaaacccgcgcgcatgattttggcggccgctttgcgtttagcctg |
| | | gaaaccagccagagcaccgcgtatctgcagatgcagaacctgaaaaccgaagat |
| | | accgcgatttatttttgcgtgcgcggcctggcggattattggggccagggcacc |
| | | aaagtgaccgtgagcagc |
| 8 | VH anti-TDP-43 | QVQLGESGPELKKPGETVKISCKASGDTFTANTMHWVKQSPGRGFKSLEWIDTS |
| | antibody MAb 9 | YSGETRAHDFGGRFAFSLETSQSTAYLQMQNLKTEDTAIYFCVRGLADYWGQGT |
| | (6B10) AA | KVTVSS |
| 9 | MAb 9 (6B10) | gcgaacaccatgcat |
| | VH CDR 1 NT | |
| 10 | MAb 9 (6B10) | ANTMH |
| | VH CDR 1 AA | |
| 11 | MAb 9 (6B10) | tggattgataccagctatagcggcgaaacccgcgcgcatgattttggcggc |
| | VH CDR 2 NT | |
| 12 | MAb 9 (6B10) | WIDTSYSGETRAHDFGG |
| | VH CDR 2 AA | |
| 13 | MAb 9 (6B10) | ggcctggcggattat |
| | VH CDR 3 NT | |
| 14 | MAb 9 (6B10) | GLADY |
| | VH CDR 3 AA | |
| 15 | LC anti-TDP-43 | gatattgaactgacccagagcccgctgaccctgagcgtgagcgcgggccagagc |
| | antibody MAb 9 | gtgaccattagctgccgcaccagccagagcattgtgcatagcaacggcgcgacc |
| | (6B10) NT | tatctggaatggtatctgcagcgcccgggctatagcccgaaactgctgatttat |
| | | ctggtgagcaacgattttagcggcgtgccgcatcgctttaccggcaccggcagc |
| | | ggcaccgattttaccctgcagattagccgcgtggaagcgcgcgatctgggcatt |
| | | tattattgctttcaggcgagctattttccgtatacctttggcggcggcacccgc |
| | | ctggaaattaaacgcgcagatgctgcaccaactgtatccatcttcccaccatcc |
| | | agtgagcagttaacatctggaggtgcctcagtcgtgtgcttcttgaacaacttc |
| | | taccccaaagacatcaatgtcaagtggaagattgatggcagtgaacgacaaaat |
| | | ggcgtcctgaacagttggactgatcaggacagcaaagacagcacctacagcatg |
| | | agcagcaccctcacgttgaccaaggacgagtatgaacgacataacagctatacc |
| | | tgtgaggccactcacaagacatcaacttcacccattgtcaagagcttcaacagg |
| | | aatgagtgt |
| 16 | LC anti-TDP-43 | DIELTQSPLTLSVSAGQSVTISCRTSQSIVHSNGATYLEWYLQRPGYSPKLLIY |
| | antibody MAb 9 | LVSNDFSGVPHRFTGTGSGTDFTLQISRVEARDLGIYYCFQASYFPYTFGGGTR |
| | (6B10) AA | LEIKRADAAPTVSIFPPSSEQLTSGGASVVCFLNNFYPKDINVKWKIDGSERQN |
| | | GVLNSWTDQDSKDSTYSMSSTLTLTKDEYERHNSYTCEATHKTSTSPIVKSFNR |
| | | NEC |
| 17 | VL anti-TDP-43 | gatattgaactgacccagagcccgctgaccctgagcgtgagcgcgggccagagc |
| | antibody MAb 9 | gtgaccattagctgccgcaccagccagagcattgtgcatagcaacggcgcgacc |
| | (6B10) NT | tatctggaatggtatctgcagcgcccgggctatagcccgaaactgctgatttat |
| | | ctggtgagcaacgattttagcggcgtgccgcatcgctttaccggcaccggcagc |
| | | ggcaccgattttaccctgcagattagccgcgtggaagcgcgcgatctgggcatt |
| | | tattattgctttcaggcgagctattttccgtatacctttggcggcggcacccgc |
| | | ctggaaattaaacgc |
| 18 | VL anti-TDP-43 | DIELTQSPLTLSVSAGQSVTISCRTSQSIVHSNGATYLEWYLQRPGYSPKLLIY |
| | antibody MAb 9 | LVSNDFSGVPHRFTGTGSGTDFTLQISRVEARDLGIYYCFQASYFPYTFGGGTR |
| | (6B10) AA | LEIKR |
| 19 | MAb 9 (6B10) | cgcaccagccagagcattgtgcatagcaacggcgcgacctatctggaa |
| | VL CDR 1 NT | |
| 20 | MAb 9 (6B10) | RTSQSIVHSNGATYLE |
| | VL CDR 1 AA | |
| 21 | MAb 9 (6B10) | ctggtgagcaacgattttagc |
| | VL CDR 2 NT | |
| 22 | MAb 9 (6B10) | LVSNDFS |
| | VL CDR 2 AA | |
| 23 | MAb 9 (6B10) | tttcaggcgagctattttccgtatacc |
| | VL CDR 3 NT | |
| 24 | MAb 9 (6B10) | FQASYFPYT |
| | VL CDR 3 AA | |
| 25 | HC anti-TDP-43 | caggtgcagctggaacagagcggcccggaactggtgcagccgggcgcgagcgtg |
| | antibody MAb 5 | aaaattagctgcaaagcgagcggcgatagctttaccgcgcagtatatgcattgg |
| | (8C5) NT | gtgaaacagagccatgtgaaaagcctggaatggaccggccatattggcccgtat |
| | | gaaggcagcaccccgtataacggcaactttaaagataaaggcagcctgaccgtg |
| | | gataaaagcagcagcaccgcgtatatggaactgcatagcctgaccagcgaagat |
| | | agcgcggtgtattattgcgcgcgcagcagcggcctggaaatttttaaaagctgg |
| | | ggccagggcaccccggtgaccgtgagcagcagcgctaaaacgacacccccatct |
| | | gtctatccactggcccctggatctgctgcccaaactaactccatggtgaccctg |
| | | ggatgcctggtcaagggctatttccctgagccagtgacagtgacctggaactct |
| | | ggatccctgtccagcggtgtgcacaccttcccagctgtcctgcagtctgacctc |
| | | tacactctgagcagctcagtgactgtcccctccagcacctggcccagcgagacc |
| | | gtcacctgcaacgttgcccacccggccagcagcaccaaggtggacaagaaaatt |
| | | gtgcccagggattgtggttgtaagccttgcatatgtacagtcccagaagtatca |
| | | tctgtcttcatcttccccccaaagcccaaggatgtgctcaccattactctgact |
| | | cctaaggtcacgtgtgttgtggtagacatcagcaaggatgatcccgaggtccag |
| | | ttcagctggtttgtagatgatgtggaggtgcacacagctcagacgcaaccccgg |
| | | gaggagcagttcaacagcactttccgctcagtcagtgaacttcccatcatgcac |
| | | caggactggctcaatggcaaggagttcaaatgcagggtcaacagtgcagctttc |
| | | cctgcccccatcgagaaaaccatctccaaaaccaaaggcagaccgaaggctcca |
| | | caggtgtacaccattccacctcccaaggagcagatggccaaggataaagtcagt |
| | | ctgacctgcatgataacagacttcttccctgaagacattactgtggagtggcag |
| | | tggaatgggcagccagcggagaactacaagaacactcagcccatcatggacaca |
| | | gatggctcttacttcgtctacagcaagctcaatgtgcagaagagcaactgggag |
| | | gcaggaaatactttcacctgctctgtgttacatgagggcctgcacaaccaccat |
| | | actgagaagagcc |
| 26 | HC anti-TDP-43 | QVQLEQSGPELVQPGASVKISCKASGDSFTAQYMHWVKQSHVKSLEWTGHIGPY |
| | antibody MAb 5 | EGSTPYNGNFKDKGSLTVDKSSSTAYMELHSLTSEDSAVYYCARSSGLEIFKSW |
| | (8C5) AA | GQGTPVTVSSSAKTTPPSVYPLAPGSAAQTNSMVTLGCLVKGYFPEPVTVTWNS |
| | | GSLSSGVHTFPAVLOSDLYTLSSSVTVPSSTWPSETVTCNVAHPASSTKVDKKI |
| | | VPRDCGCKPCICTVPEVSSVFIFPPKPKDVLTITLTPKVTCVVVDISKDDPEVQ |
| | | FSWFVDDVEVHTAQTQPREEQFNSTFRSVSELPIMHQDWLNGKEFKCRVNSAAF |
| | | PAPIEKTISKTKGRPKAPQVYTIPPPKEQMAKDKVSLTCMITDFFPEDITVEWQ |
| | | WNGQPAENYKNTQPIMDTDGSYFVYSKLNVQKSNWEAGNTFTCSVLHEGLHNHH |
| | | TEKSLSHSPGK |
| 27 | VH anti-TDP-43 | caggtgcagctggaacagagcggcccggaactggtgcagccgggcgcgagcgtg |
| | antibody MAb 5 | aaaattagctgcaaagcgagcggcgatagctttaccgcgcagtatatgcattgg |
| | (8C5) NT | gtgaaacagagccatgtgaaaagcctggaatggaccggccatattggcccgtat |
| | | gaaggcagcaccccgtataacggcaactttaaagataaaggcagcctgaccgtg |
| | | gataaaagcagcagcaccgcgtatatggaactgcatagcctgaccagcgaagat |
| | | agcgcggtgtattattgcgcgcgcagcagcggcctggaaatttttaaaagctgg |
| | | ggccagggcaccccggtgaccgtgagcagc |
| 28 | VH anti-TDP-43 | QVQLEQSGPELVQPGASVKISCKASGDSFTAQYMHWVKQSHVKSLEWTGHIGPY |
| | antibody MAb 5 | EGSTPYNGNFKDKGSLTVDKSSSTAYMELHSLTSEDSAVYYCARSSGLEIFKSW |
| | (8C5) AA | GQGTPVTVSS |
| 29 | MAb 5 (8C5) VH | gcgcagtatatgcat |
| | CDR 1 NT | |
| 30 | MAb 5 (8C5) VH | AQYMH |
| | CDR 1 AA | |
| 31 | MAb 5 (8C5) VH | tggaccggccatattggcccgtatgaaggcagcaccccgtataacggc |
| | CDR 2 NT | |
| 32 | MAb 5 (8C5) VH | WTGHIGPYEGSTPYNG |
| | CDR 2 AA | |
| 33 | MAb 5 (8C5) VH | ggcctggaaatttttaaaagc |
| | CDR 3 NT | |
| 34 | MAb 5 (8C5) VH | GLEIFKS |
| | CDR 3 AA | |
| 35 | LC anti-TDP-43 | gatattctgatgacccagagcccggcgagcctggcggtgagcctgggccagcgc |
| | antibody MAb 5 | gcgaccattagctgccgcgaaagccagagcattgtggaaagcaaaggcaacacc |
| | (8C5) NT | tatctggaatggtatctgcagaaaccgggcaaagcgccgaaactgctgatttat |
| | | gaagtgaccaaccattttagcggcgtgccgagccgctttagcggcagccgcagc |
| | | ggcaccgattttaccctgaccattagcagcctgcagccggaagattttggcatt |
| | | tattattgctttcaggaaagcaacaccccgtatacctttggccagggcaccaaa |
| | | ctggaaattaaacgcgcagatgctgcaccaactgtatccatcttcccaccatcc |
| | | agtgagcagttaacatctggaggtgcctcagtcgtgtgcttcttgaacaacttc |
| | | taccccaaagacatcaatgtcaagtggaagattgatggcagtgaacgacaaaat |
| | | ggcgtcctgaacagttggactgatcaggacagcaaagacagcacctacagcatg |
| | | agcagcaccctcacgttgaccaaggacgagtatgaacgacataacagctatacc |
| | | tgtgaggccactcacaagacatcaacttcacccattgtcaagagcttcaacagg |
| | | aatgagtgt |
| 36 | LC anti-TDP-43 | DILMTQSPASLAVSLGORATISCRESQSIVESKGNTYLEWYLQKPGKAPKLLIY |
| | antibody MAb 5 | EVTNHFSGVPSRFSGSRSGTDFTLTISSLQPEDFGIYYCFQESNTPYTFGQGTK |
| | (8C5) AA | LEIKRADAAPTVSIFPPSSEQLTSGGASVVCFLNNFYPKDINVKWKIDGSERQN |
| | | GVLNSWTDQDSKDSTYSMSSTLTLTKDEYERHNSYTCEATHKTSTSPIVKSFNR |
| | | NEC |
| 37 | VL anti-TDP-43 | gatattctgatgacccagagcccggcgagcctggcggtgagcctgggccagcgc |
| | antibody MAb 5 | gcgaccattagctgccgcgaaagccagagcattgtggaaagcaaaggcaacacc |
| | (8C5) NT | tatctggaatggtatctgcagaaaccgggcaaagcgccgaaactgctgatttat |
| | | gaagtgaccaaccattttagcggcgtgccgagccgctttagcggcagccgcagc |
| | | ggcaccgattttaccctgaccattagcagcctgcagccggaagattttggcatt |
| | | tattattgctttcaggaaagcaacaccccgtatacctttggccagggcaccaaa |
| | | ctggaaattaaacgc |
| 38 | VL anti-TDP-43 | DILMTQSPASLAVSLGQRATISCRESQSIVESKGNTYLEWYLQKPGKAPKLLIY |
| | antibody MAb 5 | EVTNHFSGVPSRFSGSRSGTDFTLTISSLQPEDFGIYYCFQESNTPYTFGQGTK |
| | (8C5) AA | LEIKR |
| 39 | MAb 5 (8C5) VL | cgcgaaagccagagcattgtggaaagcaaaggcaacacctatctggaa |
| | CDR 1 NT | |
| 40 | MAb 5 (8C5) VL | RESQSIVESKGNTYLE |
| | CDR 1 AA | |
| 41 | MAb 5 (8C5) VL | gaagtgaccaaccattttagc |
| | CDR 2 NT | |
| 42 | MAb 5 (8C5) VL | EVTNHFS |
| | CDR 2 AA | |
| 43 | MAb 5 (8C5) VL | tttcaggaaagcaacacc |
| | CDR 3 NT | |
| 44 | MAb 5 (8C5) VL | FQESNTPYT |
| | CDR 3 AA | |
| 45 | HC anti-TDP-43 | caggtgcagctgcaggaaagcgcgagcgtgctggtgcgcccgggcaccagcgtg |
| | antibody MAb 4 | cgcctgagctgcaaagcgagcgcgtatatttttaccaacagctggatgcattgg |
| | (7A1) NT | gcgaaacagcgcccgggccagggcctggaatggattggcgaaattaacccgatt |
| | | agcggcggcaccaactataacgatcagtttaaaggcaaagcgaccctgaccgtg |
| | | gataccagcagcagcaccgcgtatgtggatctgagcagcgtgaccagcgatgat |
| | | agcgcggtgtattattgcagcgaatatctgattgattggggccagggcaccacc |
| | | gtgaccgtgagcagcagcgctaaaacgacacccccatctgtctatccactggcc |
| | | cctggatctgctgcccaaactaactccatggtgaccctgggatgcctggtcaag |
| | | ggctatttccctgagccagtgacagtgacctggaactctggatccctgtccagc |
| | | ggtgtgcacaccttcccagctgtcctgcagtctgacctctacactctgagcagc |
| | | tcagtgactgtcccctccagcacctggcccagcgagaccgtcacctgcaacgtt |
| | | gcccacccggccagcagcaccaaggtggacaagaaaattgtgcccagggattgt |
| | | ggttgtaagccttgcatatgtacagtcccagaagtatcatctgtcttcatcttc |
| | | cccccaaagcccaaggatgtgctcaccattactctgactcctaaggtcacgtgt |
| | | gttgtggtagacatcagcaaggatgatcccgaggtccagttcagctggtttgta |
| | | gatgatgtggaggtgcacacagctcagacgcaaccccgggaggagcagttcaac |
| | | agcactttccgctcagtcagtgaacttcccatcatgcaccaggactggctcaat |
| | | ggcaaggagttcaaatgcagggtcaacagtgcagctttccctgcccccatcgag |
| | | aaaaccatctccaaaaccaaaggcagaccgaaggctccacaggtgtacaccatt |
| | | ccacctcccaaggagcagatggccaaggataaagtcagtctgacctgcatgata |
| | | acagacttcttccctgaagacattactgtggagtggcagtggaatgggcagcca |
| | | gcggagaactacaagaacactcagcccatcatggacacagatggctcttacttc |
| | | gtctacagcaagctcaatgtgcagaagagcaactgggaggcaggaaatactttc |
| | | acctgctctgtgttacatgagggcctgcacaaccaccatactgagaagagcctc |
| | | tcccactctcctggtaaa |
| 46 | HC anti-TDP-43 | QVQLQESASVLVRPGTSVRLSCKASAYIFTNSWMHWAKQRPGQGLEWIGEINPI |
| | antibody MAb 4 | SGGTNYNDQFKGKATLTVDTSSSTAYVDLSSVTSDDSAVYYCSEYLIDWGQGTT |
| | (7A1) AA | VTVSSSAKTTPPSVYPLAPGSAAQTNSMVTLGCLVKGYFPEPVTVTWNSGSLSS |
| | | GVHTFPAVLQSDLYTLSSSVTVPSSTWPSETVTCNVAHPASSTKVDKKIVPRDC |
| | | GCKPCICTVPEVSSVFIFPPKPKDVLTITLTPKVTCVVVDISKDDPEVQFSWFV |
| | | DDVEVHTAQTQPREEQFNSTFRSVSELPIMHQDWLNGKEFKCRVNSAAFPAPIE |
| | | KTISKTKGRPKAPQVYTIPPPKEQMAKDKVSLTCMITDFFPEDITVEWQWNGQP |
| | | AENYKNTQPIMDTDGSYFVYSKLNVQKSNWEAGNTFTCSVLHEGLHNHHTEKSL |
| | | SHSPGK |
| 47 | VH anti-TDP-43 | caggtgcagctgcaggaaagcgcgagcgtgctggtgcgcccgggcaccagcgtg |
| | antibody MAb 4 | cgcctgagctgcaaagcgagcgcgtatatttttaccaacagctggatgcattgg |
| | (7A1) NT | gcgaaacagcgcccgggccagggcctggaatggattggcgaaattaacccgatt |
| | | agcggcggcaccaactataacgatcagtttaaaggcaaagcgaccctgaccgtg |
| | | gataccagcagcagcaccgcgtatgtggatctgagcagcgtgaccagcgatgat |
| | | agcgcggtgtattattgcagcgaatatctgattgattggggccagggcaccacc |
| | | gtgaccgtgagcagc |
| 48 | VH anti-TDP-43 | QVQLQESASVLVRPGTSVRLSCKASAYIFTNSWMHWAKQRPGQGLEWIGEINPI |
| | antibody MAb 4 | SGGTNYNDQFKGKATLTVDTSSSTAYVDLSSVTSDDSAVYYCSEYLIDWGQGTT |
| | (7A1) AA | VTVSS |
| 49 | MAb 4 (7A1) VH | aacagctggatgcat |
| | CDR 1 NT | |
| 50 | MAb 4 (7A1) VH | NSWMH |
| | CDR 1 AA | |
| 51 | MAb 4 (7A1) VH | gaaattaacccgattagcggcggcaccaactataacgatcagtttaaaggc |
| | CDR 2 NT | |
| 52 | MAb 4 (7A1) VH | EINPISGGTNYNDQFKG |
| | CDR 2 AA | |
| 53 | MAb 4 (7A1) VH | agcgaatatctgattgat |
| | CDR 3 NT | |
| 54 | MAb 4 (7A1) VH | SEYLID |
| | CDR 3 AA | |
| 55 | LC anti-TDP-43 | gatattgaactgacccagagcccgctgaccctgagcgtgaccattggccagccg |
| | antibody MAb 4 | gcgagcattagctgcaaaagcgaacagagcctgctggatagcgatagcaaaacc |
| | (7A1) NT | tatctgaactggctgctgcagcgcccgggccagagcccgaaacgcctgatttat |
| | | ctggtgagcaaactggatagcggcgtgccggatcgctttaccggcagcggcagc |
| | | ggcaccgaatttaccctgaacattagccgcgtggaaaccgaagatctgggcgtg |
| | | tattattgctggcagggcacccattttcgctggacctttggcggcggcaccaaa |
| | | ctggaaattaaacgcgcagatgctgcaccaactgtatccatcttcccaccatcc |
| | | agtgagcagttaacatctggaggtgcctcagtcgtgtgcttcttgaacaacttc |
| | | taccccaaagacatcaatgtcaagtggaagattgatggcagtgaacgacaaaat |
| | | ggcgtcctgaacagttggactgatcaggacagcaaagacagcacctacagcatg |
| | | agcagcaccctcacgttgaccaaggacgagtatgaacgacataacagctatacc |
| | | tgtgaggccactcacaagacatcaacttcacccattgtcaagagcttcaacagg |
| | | aatgagtgt |
| 56 | LC anti-TDP-43 | DIELTQSPLTLSVTIGQPASISCKSEQSLLDSDSKTYLNWLLQRPGQSPKRLIY |
| | antibody MAb 4 | LVSKLDSGVPDRFTGSGSGTEFTLNISRVETEDLGVYYCWQGTHFRWTFGGGTK |
| | (7A1) AA | LEIKRADAA |
| 57 | VL anti-TDP-43 | gatattgaactgacccagagcccgctgaccctgagcgtgaccattggccagccg |
| | antibody MAb 4 | gcgagcattagctgcaaaagcgaacagagcctgctggatagcgatagcaaaacc |
| | (7A1) NT | tatctgaactggctgctgcagcgcccgggccagagcccgaaacgcctgatttat |
| | | ctggtgagcaaactggatagcggcgtgccggatcgctttaccggcagcggcagc |
| | | ggcaccgaatttaccctgaacattagccgcgtggaaaccgaagatctgggcgtg |
| | | tattattgctggcagggcacccattttcgctggacctttggcggcggcaccaaa |
| | | ctggaaattaaacgc |
| 58 | VL anti-TDP-43 | DIELTQSPLTLSVTIGQPASISCKSEQSLLDSDSKTYLNWLLQRPGQSPKRLIY |
| | antibody MAb 4 | LVSKLDSGVPDRFTGSGSGTEFTLNISRVETEDLGVYYCWQGTHFRWTFGGGTK |
| | (7A1) AA | LEIKR |
| 59 | MAb 4 (7A1) VL | aaaagcgaacagagcctgctggatagcgatagcaaaacctatctgaac |
| | CDR 1 NT | |
| 60 | MAb 4 (7A1) VL | KSEQSLLDSDSKTYLN |
| | CDR 1 AA | |
| 61 | MAb 4 (7A1) VL | ctggtgagcaaactggatagc |
| | CDR 2 NT | |
| 62 | MAb 4 (7A1) VL | LVSKLDS |
| | CDR 2 AA | |
| 63 | MAb 4 (7A1) VL | tggcagggcacccattttcgctggacc |
| | CDR 3 NT | |
| 64 | MAb 4 (7A1) VL | WQGTHFRWT |
| | CDR 3 AA | |
| 65 | HC anti-TDP-43 | caggtgaaactgcagcagagcggcccggatctggtgaaaccgggcgcgagcgtg |
| | antibody MAb 6 | aaaattagctgcaaaaccagcggctatacctttaccgaatataccatgcattgg |
| | (6B2) NT | gtgaaacagagccagggcaaaagcctggaatggattggcggcattaacccgaac |
| | | aaccgcggcaccagctataaccagaaatttaaaggcaaagcgaccctgaccgtg |
| | | gataaaagcagcagcaccgcgtatatggatctgcgcagcctgaccagcgaagat |
| | | agcgcggtgtattattgcgcgattagcagctggggccagggcaccaccgtgacc |
| | | gtgagcagcagcgctaaaacgacacccccatctgtctatccactggcccctgga |
| | | tctgctgcccaaactaactccatggtgaccctgggatgcctggtcaagggctat |
| | | ttccctgagccagtgacagtgacctggaactctggatccctgtccagcggtgtg |
| | | cacaccttcccagctgtcctgcagtctgacctctacactctgagcagctcagtg |
| | | actgtcccctccagcacctggcccagcgagaccgtcacctgcaacgttgcccac |
| | | ccggccagcagcaccaaggtggacaagaaaattgtgcccagggattgtggttgt |
| | | aagccttgcatatgtacagtcccagaagtatcatctgtcttcatcttcccccca |
| | | aagcccaaggatgtgctcaccattactctgactcctaaggtcacgtgtgttgtg |
| | | gtagacatcagcaaggatgatcccgaggtccagttcagctggtttgtagatgat |
| | | gtggaggtgcacacagctcagacgcaaccccgggaggagcagttcaacagcact |
| | | ttccgctcagtcagtgaacttcccatcatgcaccaggactggctcaatggcaag |
| | | gagttcaaatgcagggtcaacagtgcagctttccctgcccccatcgagaaaacc |
| | | atctccaaaaccaaaggcagaccgaaggctccacaggtgtacaccattccacct |
| | | cccaaggagcagatggccaaggataaagtcagtctgacctgcatgataacagac |
| | | ttcttccctgaagacattactgtggagtggcagtggaatgggcagccagcggag |
| | | aactacaagaacactcagcccatcatggacacagatggctcttacttcgtctac |
| | | agcaagctcaatgtgcagaagagcaactgggaggcaggaaatactttcacctgc |
| | | tctgtgttacatgagggcctgcacaaccaccatactgagaagagcctctcccac |
| | | tctcctggtaaa |
| 66 | HC anti-TDP-43 | QVKLQQSGPDLVKPGASVKISCKTSGYTFTEYTMHWVKQSQGKSLEWIGGINPN |
| | antibody MAb 6 | NRGTSYNQKFKGKATLTVDKSSSTAYMDLRSLTSEDSAVYYCAISSWGQGTTVT |
| | (6B2) AA | VSSSAKTTPPSVYPLAPGSAAQTNSMVTLGCLVKGYFPEPVTVTWNSGSLSSGV |
| | | HTFPAVLQSDLYTLSSSVTVPSSTWPSETVTCNVAHPASSTKVDKKIVPRDCGC |
| | | KPCICTVPEVSSVFIFPPKPKDVLTITLTPKVTCVVVDISKDDPEVQFSWFVDD |
| | | VEVHTAQTQPREEQFNSTFRSVSELPIMHQDWLNGKEFKCRVNSAAFPAPIEKT |
| | | ISKTKGRPKAPQVYTIPPPKEQMAKDKVSLTCMITDFFPEDITVEWQWNGQPAE |
| | | NYKNTQPIMDTDGSYFVYSKLNVQKSNWEAGNTFTCSVLHEGLHNHHTEKSLSH |
| | | SPGK |
| 67 | VH anti-TDP-43 | caggtgaaactgcagcagagcggcccggatctggtgaaaccgggcgcgagcgtg |
| | antibody MAb 6 | aaaattagctgcaaaaccagcggctatacctttaccgaatataccatgcattgg |
| | (6B2) NT | gtgaaacagagccagggcaaaagcctggaatggattggcggcattaacccgaac |
| | | aaccgcggcaccagctataaccagaaatttaaaggcaaagcgaccctgaccgtg |
| | | gataaaagcagcagcaccgcgtatatggatctgcgcagcctgaccagcgaagat |
| | | agcgcggtgtattattgcgcgattagcagctggggccagggcaccaccgtgacc |
| | | gtgagcagc |
| 68 | VH anti-TDP-43 | QVKLQQSGPDLVKPGASVKISCKTSGYTFTEYTMHWVKQSQGKSLEWIGGINPN |
| | antibody MAb 6 | NRGTSYNQKFKGKATLTVDKSSSTAYMDLRSLTSEDSAVYYCAISSWGQGTTVT |
| | (6B2) AA | VSS |
| 69 | MAb 4 (6B2) VH | gaatataccatgcat |
| | CDR 1 NT | |
| 70 | MAb 6 (6B2) VH | EYTMH |
| | CDR 1 AA | |
| 71 | MAb 6 (6B2) VH | ggcattaacccgaacaaccgcggcaccagctataaccagaaatttaaaggc |
| | CDR 2 NT | |
| 72 | MAb 6 (6B2) VH | GINPNNRGTSYNQKFKG |
| | CDR 2 AA | |
| 73 | MAb 6 (6B2) VH | gcgattagcagc |
| | CDR 3 NT | |
| 74 | MAb 6 (6B2) VH | AISS |
| | CDR 3 AA | |
| 75 | LC anti-TDP-43 | gatattgaactgacccagagcccgctgaccctgagcgtgattattggccagccg |
| | antibody MAb 6 | gcgagcattagctgcaaaagcagccagagcctgctgcatagcgatggcaaaacc |
| | (6B2) NT | tatctgaactggctgtttcagcgcccgggccagagcccgaaacgcctgatttat |
| | | ctggaaagcaaactggatagccgcgtgccggatcgctttaccggcagcggcagc |
| | | ggcaccgattttaccctgaaaattagccgcgtggaagcggaagatctgggcgtg |
| | | tattattgctggcagggcacccgctttccgcatacctttggcggcggcacccgc |
| | | ctggaaattaaacgcgcagatgctgcaccaactgtatccatcttcccaccatcc |
| | | agtgagcagttaacatctggaggtgcctcagtcgtgtgcttcttgaacaacttc |
| | | taccccaaagacatcaatgtcaagtggaagattgatggcagtgaacgacaaaat |
| | | ggcgtcctgaacagttggactgatcaggacagcaaagacagcacctacagcatg |
| | | agcagcaccctcacgttgaccaaggacgagtatgaacgacataacagctatacc |
| | | tgtgaggccactcacaagacatcaacttcacccattgtcaagagcttcaacagg |
| | | aatgagtgt |
| 76 | LC anti-TDP-43 | DIELTQSPLTLSVIIGQPASISCKSSQSLLHSDGKTYLNWLFQRPGQSPKRLIY |
| | antibody MAb 6 | LESKLDSRVPDRFTGSGSGTDFTLKISRVEAEDLGVYYCWQGTRFPHTFGGGTR |
| | (6B2) AA | LEIKRADAAPTVSIFPPSSEQLTSGGASVVCFLNNFYPKDINVKWKIDGSERQN |
| | | GVLNSWTDQDSKDSTYSMSSTLTLTKDEYERHNSYTCEATHKTSTSPIVKSFNR |
| | | NEC |
| 77 | VL anti-TDP-43 | gatattgaactgacccagagcccgctgaccctgagcgtgattattggccagccg |
| | antibody MAb 6 | gcgagcattagctgcaaaagcagccagagcctgctgcatagcgatggcaaaacc |
| | (6B2) NT | tatctgaactggctgtttcagcgcccgggccagagcccgaaacgcctgatttat |
| | | ctggaaagcaaactggatagccgcgtgccggatcgctttaccggcagcggcagc |
| | | ggcaccgattttaccctgaaaattagccgcgtggaagcggaagatctgggcgtg |
| | | tattattgctggcagggcacccgctttccgcatacctttggcggcggcacccgc |
| | | ctggaaattaaacgc |
| 78 | VL anti-TDP-43 | DIELTQSPLTLSVIIGQPASISCKSSQSLLHSDGKTYLNWLFQRPGQSPKRLIY |
| | antibody MAb 6 | LESKLDSRVPDRFTGSGSGTDFTLKISRVEAEDLGVYYCWQGTRFPHTFGGGTR |
| | (6B2) AA | LEIKR |
| 79 | MAb 6 (6B2) VL | aaaagcagccagagcctgctgcatagcgatggcaaaacctatctgaac |
| | CDR 1 NT | |
| 80 | MAb 6 (6B2) VL | KSSQSLLHSDGKTYLN |
| | CDR 1 AA | |
| 81 | MAb 6 (6B2) VL | ctggaaagcaaactggatagc |
| | CDR 2 NT | |
| 82 | MAb 6 (6B2) VL | LESKLDS |
| | CDR 2 AA | |
| 83 | MAb 6 (6B2) VL | tggcagggcacccgctttccgcatacc |
| | CDR 3 NT | |
| 84 | MAb 6 (6B2) VL | WQGTRFPHT |
| | CDR 3 AA | |
| 85 | Detection | MNFGAFSINPAMMAAAQAALQSSWGMMGMLASQQNQSGPSGNNQNQGNMQ- |
| | Peptide TDP-43 | [PEG4]-MDSKS^{p}S^{p}GWGMNH |
| | No. 9 and TDP- | Note: PEG4K linker between Q50 and M51, S55 and S56 |
| | 43 No. 5 pair AA | are phosphoserines |

1. An isolated antibody or antigen-binding fragment thereof capable of binding to a TAR DNA-binding protein 43 (TDP-43) protein or fragment thereof, the antibody or antigen-binding fragment thereof comprising an immunoglobulin heavy chain (HC) variable domain sequence and an immunoglobulin light chain (LC) variable domain sequence, wherein:
the immunoglobulin HC variable domain sequence comprises:
(i) an HC CDR1 comprising an amino acid sequence having at least 95-99% identity to SEQ ID NO: 10,
(ii) an HC CDR2 comprising an amino acid sequence having at least 95-99% identity to SEQ ID NO: 12, and
(iii) an HC CDR3 comprising an amino acid sequence having at least 95-99% identity to SEQ ID NO: 14; and
the immunoglobulin LC variable domain sequence comprises:
(i) an LC CDR1 comprising an amino acid sequence having at least 95-99% identity to SEQ ID NO: 20,
(ii) an LC CDR2 comprising an amino acid sequence having at least 95-99% identity to SEQ ID NO: 22, and
(iii) an LC CDR3 comprising an amino acid sequence having at least 95-99% identity to SEQ ID NO: 24;
the immunoglobulin HC variable domain sequence comprises:
(i) an HC CDR1 comprising an amino acid sequence having at least 95-99% identity to SEQ ID NO: 30,
(ii) an HC CDR2 comprising an amino acid sequence having at least 95-99% identity to SEQ ID NO: 32, and
(iii) an HC CDR3 comprising an amino acid sequence having at least 95-99% identity to SEQ ID NO: 34; and
the immunoglobulin LC variable domain sequence comprises:
(i) an LC CDR1 comprising an amino acid sequence having at least 95-99% identity to SEQ ID NO: 40,
(ii) an LC CDR2 comprising an amino acid sequence having at least 95-99% identity to SEQ ID NO: 42, and
(iii) an LC CDR3 comprising an amino acid sequence having at least 95-99% identity to SEQ ID NO: 44;
the immunoglobulin HC variable domain sequence comprises:
(i) an HC CDR1 comprising an amino acid sequence having at least 95-99% identity to SEQ ID NO: 50,
(ii) an HC CDR2 comprising an amino acid sequence having at least 95-99% identity to SEQ ID NO: 52, and
(iii) an HC CDR3 comprising an amino acid sequence having at least 95-99% identity to SEQ ID NO: 54; and
the immunoglobulin LC variable domain sequence comprises:
(i) an LC CDR1 comprising an amino acid sequence having at least 95-99% identity to SEQ ID NO: 60,
(ii) an LC CDR2 comprising an amino acid sequence having at least 95-99% identity to SEQ ID NO: 62, and
(iii) an LC CDR3 comprising an amino acid sequence having at least 95-99% identity to SEQ ID NO: 64; or
the immunoglobulin HC variable domain sequence comprises:
(i) an HC CDR1 comprising an amino acid sequence having at least 95-99% identity to SEQ ID NO: 70,
(ii) an HC CDR2 comprising an amino acid sequence having at least 95-99% identity to SEQ ID NO: 72, and
(iii) an HC CDR3 comprising an amino acid sequence having at least 95-99% identity to SEQ ID NO: 74; and
the immunoglobulin LC variable domain sequence comprises:
(i) an LC CDR1 comprising an amino acid sequence having at least 95-99% identity to SEQ ID NO: 80,
(ii) an LC CDR2 comprising an amino acid sequence having at least 95-99% identity to SEQ ID NO: 82, and
(iii) an LC CDR3 comprising an amino acid sequence having at least 95-99% identity to SEQ ID NO: 84.

2. The isolated antibody or antigen-binding fragment thereof of claim 1, wherein:
the immunoglobulin HC variable domain sequence comprises:
(i) an HC CDR1 comprising the amino acid sequence of SEQ ID NO: 10,
(ii) an HC CDR2 comprising the amino acid sequence of SEQ ID NO: 12, and
(iii) an HC CDR3 comprising the amino acid sequence of SEQ ID NO: 14; and
the immunoglobulin LC variable domain sequence comprises:
(i) an LC CDR1 comprising the amino acid sequence of SEQ ID NO: 20,
(ii) an LC CDR2 comprising the amino acid sequence of SEQ ID NO: 22, and
(iii) an LC CDR3 comprising the amino acid sequence of SEQ ID NO: 24;
the immunoglobulin HC variable domain sequence comprises:
(i) an HC CDR1 comprising the amino acid sequence of SEQ ID NO: 30,
(ii) an HC CDR2 comprising the amino acid sequence of SEQ ID NO: 32, and
(iii) an HC CDR3 comprising the amino acid sequence of SEQ ID NO: 34; and
the immunoglobulin LC variable domain sequence comprises:
(i) an LC CDR1 comprising the amino acid sequence of SEQ ID NO: 40,
(ii) an LC CDR2 comprising the amino acid sequence of SEQ ID NO: 42, and
(iii) an LC CDR3 comprising the amino acid sequence of SEQ ID NO: 44;
the immunoglobulin HC variable domain sequence comprises:
(i) an HC CDR1 comprising the amino acid sequence of SEQ ID NO: 50,
(ii) an HC CDR2 comprising the amino acid sequence of SEQ ID NO: 52, and
(iii) an HC CDR3 comprising the amino acid sequence of SEQ ID NO: 54; and
the immunoglobulin LC variable domain sequence comprises:
(i) an LC CDR1 comprising the amino acid sequence of SEQ ID NO: 60,
(ii) an LC CDR2 comprising the amino acid sequence of SEQ ID NO: 62, and
(iii) an LC CDR3 comprising the amino acid sequence of SEQ ID NO: 64; or
the immunoglobulin HC variable domain sequence comprises:
(i) an HC CDR1 comprising the amino acid sequence of SEQ ID NO: 70,
(ii) an HC CDR2 comprising the amino acid sequence of SEQ ID NO: 72, and
(iii) an HC CDR3 comprising the amino acid sequence of SEQ ID NO: 74; and
the immunoglobulin LC variable domain sequence comprises:
(i) an LC CDR1 comprising the amino acid sequence of SEQ ID NO: 80,
(ii) an LC CDR2 comprising the amino acid sequence of SEQ ID NO: 82, and
(iii) an LC CDR3 comprising the amino acid sequence of SEQ ID NO: 84.

3. The isolated antibody or antigen-binding fragment thereof of claim 1, wherein:
the immunoglobulin HC variable domain sequence comprises an amino acid sequence having at least 95-99% identity to SEQ ID NO: 8, and the immunoglobulin LC variable domain sequence comprises an amino acid sequence having at least 95-99% identity to SEQ ID NO: 18;
the immunoglobulin HC variable domain sequence comprises an amino acid sequence having at least 95-99% identity to SEQ ID NO: 28, and the immunoglobulin LC variable domain sequence comprises an amino acid sequence having at least 95-99% identity to SEQ ID NO: 38;
the immunoglobulin HC variable domain sequence comprises an amino acid sequence having at least 95-99% identity to SEQ ID NO: 48, and the immunoglobulin LC variable domain sequence comprises an amino acid sequence having at least 95-99% identity to SEQ ID NO: 58; or
the immunoglobulin HC variable domain sequence comprises an amino acid sequence having at least 95-99% identity to SEQ ID NO: 68, and the immunoglobulin LC variable domain sequence comprises an amino acid sequence having at least 95-99% identity to SEQ ID NO: 78.

4. The isolated antibody or antigen-binding fragment thereof of claim 1, wherein:
the immunoglobulin HC variable domain sequence comprises the amino acid sequence of SEQ ID NO: 8, and the immunoglobulin LC variable domain sequence comprises the amino acid sequence of SEQ ID NO: 18;
the immunoglobulin HC variable domain sequence comprises the amino acid sequence of SEQ ID NO: 28, and the immunoglobulin LC variable domain sequence comprises the amino acid sequence of SEQ ID NO: 38;
the immunoglobulin HC variable domain sequence comprises the amino acid sequence of SEQ ID NO: 48, and the immunoglobulin LC variable domain sequence comprises the amino acid sequence of SEQ ID NO: 58; or
the immunoglobulin HC variable domain sequence comprises the amino acid sequence of SEQ ID NO: 68, and the immunoglobulin LC variable domain sequence comprises the amino acid sequence of SEQ ID NO: 78.

5. The isolated antibody or antigen-binding fragment thereof of claim 1, wherein:
the immunoglobulin HC variable domain sequence is encoded by a nucleic acid comprising the nucleotide sequence of SEQ ID NO: 7, and the immunoglobulin LC variable domain sequence is encoded by a nucleic acid comprising the nucleotide sequence of SEQ ID NO: 17;
the immunoglobulin HC variable domain sequence is encoded by a nucleic acid comprising the nucleotide sequence of SEQ ID NO: 27, and the immunoglobulin LC variable domain sequence is encoded by a nucleic acid comprising the nucleotide sequence of SEQ ID NO: 37;
the immunoglobulin HC variable domain sequence is encoded by a nucleic acid comprising the nucleotide sequence of SEQ ID NO: 47, and the immunoglobulin LC variable domain sequence is encoded by a nucleic acid comprising the nucleotide sequence of SEQ ID NO: 57; or
the immunoglobulin HC variable domain sequence is encoded by a nucleic acid comprising the nucleotide sequence of SEQ ID NO: 67, and the immunoglobulin LC variable domain sequence is encoded by a nucleic acid comprising the nucleotide sequence of SEQ ID NO: 77.

6. The isolated antibody or antigen-binding fragment thereof of claim 1, wherein the isolated antibody or antigen-binding fragment thereof comprises:
a heavy chain comprising an amino acid sequence having at least 95-99% identity to SEQ ID NO: 6, and a light chain comprising an amino acid sequence having at least 95-99% identity to SEQ ID NO: 16;
a heavy chain comprising an amino acid sequence having at least 95-99% identity to SEQ ID NO: 26, and a light chain comprising an amino acid sequence having at least 95-99% identity to SEQ ID NO: 36;
a heavy chain comprising an amino acid sequence having at least 95-99% identity to SEQ ID NO: 46, and a light chain comprising an amino acid sequence having at least 95-99% identity to SEQ ID NO: 56; or
a heavy chain comprising an amino acid sequence having at least 95-99% identity to SEQ ID NO: 66, and a light chain comprising an amino acid sequence having at least 95-99% identity to SEQ ID NO: 76.

7. The isolated antibody or antigen-binding fragment thereof of claim 1, wherein the isolated antibody or antigen-binding fragment thereof comprises:
a heavy chain comprising the amino acid sequence of SEQ ID NO: 6, and a light chain comprising the amino acid sequence of SEQ ID NO: 16;
a heavy chain comprising the amino acid sequence of SEQ ID NO: 26, and a light chain comprising the amino acid sequence of SEQ ID NO: 36;
a heavy chain comprising the amino acid sequence of SEQ ID NO: 46, and a light chain comprising the amino acid sequence of SEQ ID NO: 56; or
a heavy chain comprising the amino acid sequence of SEQ ID NO: 66, and a light chain comprising the amino acid sequence of SEQ ID NO: 76.

8. The isolated antibody or antigen-binding fragment thereof of claim 1, wherein the isolated antibody or antigen-binding fragment thereof comprises:
a heavy chain encoded by a nucleic acid comprising the nucleotide sequence of SEQ ID NO: 5, and a light chain encoded by a nucleic acid comprising the nucleotide sequence of SEQ ID NO: 15;
a heavy chain encoded by a nucleic acid comprising the nucleotide sequence of SEQ ID NO: 25, and a light chain encoded by a nucleic acid comprising the nucleotide sequence of SEQ ID NO: 35;
a heavy chain encoded by a nucleic acid comprising the nucleotide sequence of SEQ ID NO: 45, and a light chain encoded by a nucleic acid comprising the nucleotide sequence of SEQ ID NO: 55; or
a heavy chain encoded by a nucleic acid comprising the nucleotide sequence of SEQ ID NO: 65, and a light chain encoded by a nucleic acid comprising the nucleotide sequence of SEQ ID NO: 75.

9. The isolated antibody or antigen-binding fragment thereof of claim 1, wherein the isolated antibody or antigen-binding fragment thereof immunospecifically binds to an epitope of a TDP-43 protein or fragment thereof.

10. The isolated antibody or antigen-binding fragment thereof of claim 1, wherein the isolated antibody or antigen-binding fragment thereof immunospecifically binds to at least a portion of the TDP-43 protein of SEQ ID NO: 2.

11. The isolated antibody or antigen-binding fragment thereof of claim 1, wherein the isolated antibody or antigen-binding fragment thereof immunospecifically binds to a pathologic TDP-43 protein or fragment thereof.

12. An isolated nucleic acid comprising a nucleotide sequence encoding the antibody or antigen-binding fragment thereof of claim 1.

13. A vector comprising the isolated nucleic acid of claim 12.

14. A cell comprising the vector of claim 13.

15. An immunoassay comprising one or more of the antibodies or antigen-binding fragments thereof of claim 1.

16-40. (canceled)

41. A method for isolating and detecting a protein biomarker in a biological sample comprising extracellular vesicles (EVs), the method comprising:
treating the sample with a solubilizing reagent comprising about 1-3% sodium dodecyl sulfate (SDS) detergent to lyse the EVs; and
detecting a presence or absence of the protein biomarker based on a measured binding or lack of binding of the sample to one or more antibodies or antigen-binding fragments thereof that are specific to the protein biomarker.

42. The method of claim 41, wherein the sample is treated with the solubilizing reagent at room temperature for a period of time of about 5 minutes to about 15 minutes.

43. The method of claim 41, further comprising centrifuging the sample.

44. The method of claim 41, further comprising subjecting the sample to one or more freeze-thaw cycles.

45-49. (canceled)

50. An immunoassay kit for selectively detecting a TAR DNA-binding protein 43 (TDP-43) protein or fragment thereof in a biological sample, the kit comprising:
one or more antibodies or antigen-binding fragments thereof capable of binding to a TDP-43 protein or fragment thereof, the antibodies or antigen-binding fragments thereof comprising an immunoglobulin heavy chain (HC) variable domain sequence and an immunoglobulin light chain (LC) variable domain sequence, wherein:
the immunoglobulin HC variable domain sequence comprises:
(i) an HC CDR1 comprising an amino acid sequence having at least 95-99% identity to SEQ ID NO: 10,
(ii) an HC CDR2 comprising an amino acid sequence having at least 95-99% identity to SEQ ID NO: 12, and
(iii) an HC CDR3 comprising an amino acid sequence having at least 95-99% identity to SEQ ID NO: 14; and
the immunoglobulin LC variable domain sequence comprises:
(i) an LC CDR1 comprising an amino acid sequence having at least 95-99% identity to SEQ ID NO: 20,
(ii) an LC CDR2 comprising an amino acid sequence having at least 95-99% identity to SEQ ID NO: 22, and
(iii) an LC CDR3 comprising an amino acid sequence having at least 95-99% identity to SEQ ID NO: 24;
the immunoglobulin HC variable domain sequence comprises:
(i) an HC CDR1 comprising an amino acid sequence having at least 95-99% identity to SEQ ID NO: 30,
(ii) an HC CDR2 comprising an amino acid sequence having at least 95-99% identity to SEQ ID NO: 32, and
(iii) an HC CDR3 comprising an amino acid sequence having at least 95-99% identity to SEQ ID NO: 34; and
the immunoglobulin LC variable domain sequence comprises:
(i) an LC CDR1 comprising an amino acid sequence having at least 95-99% identity to SEQ ID NO: 40,
(ii) an LC CDR2 comprising an amino acid sequence having at least 95-99% identity to SEQ ID NO: 42, and
(iii) an LC CDR3 comprising an amino acid sequence having at least 95-99% identity to SEQ ID NO: 44;
the immunoglobulin HC variable domain sequence comprises:
(i) an HC CDR1 comprising an amino acid sequence having at least 95-99% identity to SEQ ID NO: 50,
(ii) an HC CDR2 comprising an amino acid sequence having at least 95-99% identity to SEQ ID NO: 52, and
(iii) an HC CDR3 comprising an amino acid sequence having at least 95-99% identity to SEQ ID NO: 54; and
the immunoglobulin LC variable domain sequence comprises:
(i) an LC CDR1 comprising an amino acid sequence having at least 95-99% identity to SEQ ID NO: 60,
(ii) an LC CDR2 comprising an amino acid sequence having at least 95-99% identity to SEQ ID NO: 62, and
(iii) an LC CDR3 comprising an amino acid sequence having at least 95-99% identity to SEQ ID NO: 64; or
the immunoglobulin HC variable domain sequence comprises:
(i) an HC CDR1 comprising an amino acid sequence having at least 95-99% identity to SEQ ID NO: 70,
(ii) an HC CDR2 comprising an amino acid sequence having at least 95-99% identity to SEQ ID NO: 72, and
(iii) an HC CDR3 comprising an amino acid sequence having at least 95-99% identity to SEQ ID NO: 74; and
the immunoglobulin LC variable domain sequence comprises:
(i) an LC CDR1 comprising an amino acid sequence having at least 95-99% identity to SEQ ID NO: 80,
(ii) an LC CDR2 comprising an amino acid sequence having at least 95-99% identity to SEQ ID NO: 82, and
(iii) an LC CDR3 comprising an amino acid sequence having at least 95-99% identity to SEQ ID NO: 84;
a detection reagent;
optionally, buffers and receptacles; and
optionally, one or more of packaging or instruction for use.

51-56. (canceled)


SEQUENCE LISTING (85 sequences)

SEQ ID NO: 1 - DNA, 1242 bp, synthetic construct
  source 1..1242: mol_type = other DNA
atgtctgaatatattcgggtaaccgaagatgagaacgatgagcccattgaaataccatcggaagacgatgggacggtgctgctctccacggttacagcccagtttccaggggcgtgtgggcttcgctacaggaatccagtgtctcagtgtatgagaggtgtccggctggtagaaggaattctgcatgccccagatgctggctggggaaatctggtgtatgttgtcaactatccaaaagataacaaaagaaaaatggatgagacagatgcttcatcagcagtgaaagtgaaaagagcagtccagaaaacatccgatttaatagtgttgggtctcccatggaaaacaaccgaacaggacctgaaagagtattttagtacctttggagaagttcttatggtgcaggtcaagaaagatcttaagactggtcattcaaaggggtttggctttgttcgttttacggaatatgaaacacaagtgaaagtaatgtcacagcgacatatgatagatggacgatggtgtgactgcaaacttcctaattctaagcaaagccaagatgagcctttgagaagcagaaaagtgtttgtggggcgctgtacagaggacatgactgaggatgagctgcgggagttcttctctcagtacggggatgtgatggatgtcttcatccccaagccattcagggcctttgcctttgttacatttgcagatgatcagattgcgcagtctctttgtggagaggacttgatcattaaaggaatcagcgttcatatatccaatgccgaacctaagcacaatagcaatagacagttagaaagaagtggaagatttggtggtaatccaggtggctttgggaatcagggtggatttggtaatagcagagggggtggagctggtttgggaaacaatcaaggtagtaatatgggtggtgggatgaactttggtgcgttcagcattaatccagccatgatggctgccgcccaggcagcactacagagcagttggggtatgatgggcatgttagccagccagcagaaccagtcaggcccatcgggtaataaccaaaaccaaggcaacatgcagagggagccaaaccaggccttcggttctggaaataactcttatagtggctctaattctggtgcagcaattggttggggatcagcatccaatgcagggtcgggcagtggttttaatggaggctttggctcaagcatggattctaagtcttctggctggggaatg

SEQ ID NO: 2 - protein, 416 aa, synthetic construct [sequence omitted]

SEQ ID NO: 3 - DNA, 303 bp, synthetic construct
  source 1..303: mol_type = other DNA
gccgaacctaagcacaatagcaatagacagttagaaagaagtggaagatttggtggtaatccaggtggctttgggaatcagggtggatttggtaatagcagagggggtggagctggtttgggaaacaatcaaggtagtaatatgggtggtgggatgaactttggtgcgttcagcattaatccagccatgatggctgccgcccaggcagcactacagagcagttggggtatgatgggcatgttagccagccagcagaaccagtcaggcccatcgggtaataaccaaaaccaaggcaacatgcag

SEQ ID NO: 4 - protein, 101 aa, synthetic construct
AEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQ

SEQ ID NO: 5 - DNA, 1317 bp, synthetic construct
  source 1..1317: mol_type = other DNA
caggtgcagctgggcgaaagcggcccggaactgaaaaaaccgggcgaaaccgtgaaaattagctgcaaagcgagcggcgatacctttaccgcgaacaccatgcattgggtgaaacagagcccgggccgcggctttaaaagcctggaatggattgataccagctatagcggcgaaacccgcgcgcatgattttggcggccgctttgcgtttagcctggaaaccagccagagcaccgcgtatctgcagatgcagaacctgaaaaccgaagataccgcgatttatttttgcgtgcgcggcctggcggattattggggccagggcaccaaagtgaccgtgagcagcagcgctaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaa

SEQ ID NO: 6 - protein, 439 aa, synthetic construct
QVQLGESGPELKKPGETVKISCKASGDTFTANTMHWVKQSPGRGFKSLEWIDTSYSGETRAHDFGGRFAFSLETSQSTAYLQMQNLKTEDTAIYFCVRGLADYWGQGTKVTVSSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 7 - DNA, 342 bp, synthetic construct
  source 1..342: mol_type = other DNA
caggtgcagctgggcgaaagcggcccggaactgaaaaaaccgggcgaaaccgtgaaaattagctgcaaagcgagcggcgatacctttaccgcgaacaccatgcattgggtgaaacagagcccgggccgcggctttaaaagcctggaatggattgataccagctatagcggcgaaacccgcgcgcatgattttggcggccgctttgcgtttagcctggaaaccagccagagcaccgcgtatctgcagatgcagaacctgaaaaccgaagataccgcgatttatttttgcgtgcgcggcctggcggattattggggccagggcaccaaagtgaccgtgagcagc

SEQ ID NO: 8 - protein, 114 aa, synthetic construct
QVQLGESGPELKKPGETVKISCKASGDTFTANTMHWVKQSPGRGFKSLEWIDTSYSGETRAHDFGGRFAFSLETSQSTAYLQMQNLKTEDTAIYFCVRGLADYWGQGTKVTVSS

SEQ ID NO: 9 - DNA, 15 bp, synthetic construct
  source 1..15: mol_type = other DNA
gcgaacaccatgcat

SEQ ID NO: 10 - protein, 5 aa, synthetic construct
ANTMH

SEQ ID NO: 11 - DNA, 51 bp, synthetic construct
  source 1..51: mol_type = other DNA
tggattgataccagctatagcggcgaaacccgcgcgcatgattttggcggc

SEQ ID NO: 12 - protein, 17 aa, synthetic construct
WIDTSYSGETRAHDFGG

SEQ ID NO: 13 - DNA, 15 bp, synthetic construct
  source 1..15: mol_type = other DNA
ggcctggcggattat

SEQ ID NO: 14 - protein, 5 aa, synthetic construct
GLADY

SEQ ID NO: 15 - DNA, 657 bp, synthetic construct
  source 1..657: mol_type = other DNA
gatattgaactgacccagagcccgctgaccctgagcgtgagcgcgggccagagcgtgaccattagctgccgcaccagccagagcattgtgcatagcaacggcgcgacctatctggaatggtatctgcagcgcccgggctatagcccgaaactgctgatttatctggtgagcaacgattttagcggcgtgccgcatcgctttaccggcaccggcagcggcaccgattttaccctgcagattagccgcgtggaagcgcgcgatctgggcatttattattgctttcaggcgagctattttccgtatacctttggcggcggcacccgcctggaaattaaacgcgcagatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgt

SEQ ID NO: 16 - protein, 219 aa, synthetic construct
DIELTQSPLTLSVSAGQSVTISCRTSQSIVHSNGATYLEWYLQRPGYSPKLLIYLVSNDFSGVPHRFTGTGSGTDFTLQISRVEARDLGIYYCFQASYFPYTFGGGTRLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 17 - DNA, 339 bp, synthetic construct
  source 1..339: mol_type = other DNA
gatattgaactgacccagagcccgctgaccctgagcgtgagcgcgggccagagcgtgaccattagctgccgcaccagccagagcattgtgcatagcaacggcgcgacctatctggaatggtatctgcagcgcccgggctatagcccgaaactgctgatttatctggtgagcaacgattttagcggcgtgccgcatcgctttaccggcaccggcagcggcaccgattttaccctgcagattagccgcgtggaagcgcgcgatctgggcatttattattgctttcaggcgagctattttccgtatacctttggcggcggcacccgcctggaaattaaacgc

SEQ ID NO: 18 - protein, 113 aa, synthetic construct
DIELTQSPLTLSVSAGQSVTISCRTSQSIVHSNGATYLEWYLQRPGYSPKLLIYLVSNDFSGVPHRFTGTGSGTDFTLQISRVEARDLGIYYCFQASYFPYTFGGGTRLEIKR

SEQ ID NO: 19 - DNA, 48 bp, synthetic construct
  source 1..48: mol_type = other DNA
cgcaccagccagagcattgtgcatagcaacggcgcgacctatctggaa

SEQ ID NO: 20 - protein, 16 aa, synthetic construct
RTSQSIVHSNGATYLE

SEQ ID NO: 21 - DNA, 21 bp, synthetic construct
  source 1..21: mol_type = other DNA
ctggtgagcaacgattttagc

SEQ ID NO: 22 - protein, 7 aa, synthetic construct
LVSNDFS

SEQ ID NO: 23 - DNA, 27 bp, synthetic construct
  source 1..27: mol_type = other DNA
tttcaggcgagctattttccgtatacc

SEQ ID NO: 24 - protein, 9 aa, synthetic construct
FQASYFPYT

SEQ ID NO: 25 - DNA, 1309 bp, synthetic construct
  source 1..1309: mol_type = other DNA
caggtgcagctggaacagagcggcccggaactggtgcagccgggcgcgagcgtgaaaattagctgcaaagcgagcggcgatagctttaccgcgcagtatatgcattgggtgaaacagagccatgtgaaaagcctggaatggaccggccatattggcccgtatgaaggcagcaccccgtataacggcaactttaaagataaaggcagcctgaccgtggataaaagcagcagcaccgcgtatatggaactgcatagcctgaccagcgaagatagcgcggtgtattattgcgcgcgcagcagcggcctggaaatttttaaaagctggggccagggcaccccggtgaccgtgagcagcagcgctaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcc

SEQ ID NO: 26 - protein, 443 aa, synthetic construct
QVQLEQSGPELVQPGASVKISCKASGDSFTAQYMHWVKQSHVKSLEWTGHIGPYEGSTPYNGNFKDKGSLTVDKSSSTAYMELHSLTSEDSAVYYCARSSGLEIFKSWGQGTPVTVSSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 27 - DNA, 354 bp, synthetic construct
  source 1..354: mol_type = other DNA
caggtgcagctggaacagagcggcccggaactggtgcagccgggcgcgagcgtgaaaattagctgcaaagcgagcggcgatagctttaccgcgcagtatatgcattgggtgaaacagagccatgtgaaaagcctggaatggaccggccatattggcccgtatgaaggcagcaccccgtataacggcaactttaaagataaaggcagcctgaccgtggataaaagcagcagcaccgcgtatatggaactgcatagcctgaccagcgaagatagcgcggtgtattattgcgcgcgcagcagcggcctggaaatttttaaaagctggggccagggcaccccggtgaccgtgagcagc

SEQ ID NO: 28 - protein, 118 aa, synthetic construct
QVQLEQSGPELVQPGASVKISCKASGDSFTAQYMHWVKQSHVKSLEWTGHIGPYEGSTPYNGNFKDKGSLTVDKSSSTAYMELHSLTSEDSAVYYCARSSGLEIFKSWGQGTPVTVSS

SEQ ID NO: 29 - DNA, 15 bp, synthetic construct
  source 1..15: mol_type = other DNA
gcgcagtatatgcat

SEQ ID NO: 30 - protein, 5 aa, synthetic construct
AQYMH

SEQ ID NO: 31 - DNA, 48 bp, synthetic construct
  source 1..48: mol_type = other DNA
tggaccggccatattggcccgtatgaaggcagcaccccgtataacggc

SEQ ID NO: 32 - protein, 16 aa, synthetic construct
WTGHIGPYEGSTPYNG

SEQ ID NO: 33 - DNA, 21 bp, synthetic construct
  source 1..21: mol_type = other DNA
ggcctggaaatttttaaaagc

SEQ ID NO: 34 - protein, 7 aa, synthetic construct
GLEIFKS

SEQ ID NO: 35 - DNA, 657 bp, synthetic construct
  source 1..657: mol_type = other DNA
gatattctgatgacccagagcccggcgagcctggcggtgagcctgggccagcgcgcgaccattagctgccgcgaaagccagagcattgtggaaagcaaaggcaacacctatctggaatggtatctgcagaaaccgggcaaagcgccgaaactgctgatttatgaagtgaccaaccattttagcggcgtgccgagccgctttagcggcagccgcagcggcaccgattttaccctgaccattagcagcctgcagccggaagattttggcatttattattgctttcaggaaagcaacaccccgtatacctttggccagggcaccaaactggaaattaaacgcgcagatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgt

SEQ ID NO: 36 - protein, 219 aa, synthetic construct
DILMTQSPASLAVSLGQRATISCRESQSIVESKGNTYLEWYLQKPGKAPKLLIYEVTNHFSGVPSRFSGSRSGTDFTLTISSLQPEDFGIYYCFQESNTPYTFGQGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 37 - DNA, 339 bp, synthetic construct
  source 1..339: mol_type = other DNA
gatattctgatgacccagagcccggcgagcctggcggtgagcctgggccagcgcgcgaccattagctgccgcgaaagccagagcattgtggaaagcaaaggcaacacctatctggaatggtatctgcagaaaccgggcaaagcgccgaaactgctgatttatgaagtgaccaaccattttagcggcgtgccgagccgctttagcggcagccgcagcggcaccgattttaccctgaccattagcagcctgcagccggaagattttggcatttattattgctttcaggaaagcaacaccccgtatacctttggccagggcaccaaactggaaattaaacgc

SEQ ID NO: 38 - protein, 113 aa, synthetic construct
DILMTQSPASLAVSLGQRATISCRESQSIVESKGNTYLEWYLQKPGKAPKLLIYEVTNHFSGVPSRFSGSRSGTDFTLTISSLQPEDFGIYYCFQESNTPYTFGQGTKLEIKR

SEQ ID NO: 39 - DNA, 48 bp, synthetic construct
  source 1..48: mol_type = other DNA
cgcgaaagccagagcattgtggaaagcaaaggcaacacctatctggaa

SEQ ID NO: 40 - protein, 16 aa, synthetic construct
RESQSIVESKGNTYLE

SEQ ID NO: 41 - DNA, 21 bp, synthetic construct
  source 1..21: mol_type = other DNA
gaagtgaccaaccattttagc

SEQ ID NO: 42 - protein, 7 aa, synthetic construct
EVTNHFS

SEQ ID NO: 43 - DNA, 18 bp, synthetic construct
  source 1..18: mol_type = other DNA
tttcaggaaagcaacacc

SEQ ID NO: 44 - protein, 9 aa, synthetic construct
FQESNTPYT

SEQ ID NO: 45 - DNA, 1314 bp, synthetic construct
  source 1..1314: mol_type = other DNA
caggtgcagctgcaggaaagcgcgagcgtgctggtgcgcccgggcaccagcgtgcgcctgagctgcaaagcgagcgcgtatatttttaccaacagctggatgcattgggcgaaacagcgcccgggccagggcctggaatggattggcgaaattaacccgattagcggcggcaccaactataacgatcagtttaaaggcaaagcgaccctgaccgtggataccagcagcagcaccgcgtatgtggatctgagcagcgtgaccagcgatgatagcgcggtgtattattgcagcgaatatctgattgattggggccagggcaccaccgtgaccgtgagcagcagcgctaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaa

SEQ ID NO: 46 - protein, 438 aa, synthetic construct
QVQLQESASVLVRPGTSVRLSCKASAYIFTNSWMHWAKQRPGQGLEWIGEINPISGGTNYNDQFKGKATLTVDTSSSTAYVDLSSVTSDDSAVYYCSEYLIDWGQGTTVTVSSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 47 - DNA, 339 bp, synthetic construct
  source 1..339: mol_type = other DNA
caggtgcagctgcaggaaagcgcgagcgtgctggtgcgcccgggcaccagcgtgcgcctgagctgcaaagcgagcgcgtatatttttaccaacagctggatgcattgggcgaaacagcgcccgggccagggcctggaatggattggcgaaattaacccgattagcggcggcaccaactataacgatcagtttaaaggcaaagcgaccctgaccgtggataccagcagcagcaccgcgtatgtggatctgagcagcgtgaccagcgatgatagcgcggtgtattattgcagcgaatatctgattgattggggccagggcaccaccgtgaccgtgagcagc

SEQ ID NO: 48 - protein, 113 aa, synthetic construct
QVQLQESASVLVRPGTSVRLSCKASAYIFTNSWMHWAKQRPGQGLEWIGEINPISGGTNYNDQFKGKATLTVDTSSSTAYVDLSSVTSDDSAVYYCSEYLIDWGQGTTVTVSS

SEQ ID NO: 49 - DNA, 15 bp, synthetic construct
  source 1..15: mol_type = other DNA
aacagctggatgcat

SEQ ID NO: 50 - protein, 5 aa, synthetic construct
NSWMH

SEQ ID NO: 51 - DNA, 51 bp, synthetic construct
  source 1..51: mol_type = other DNA
gaaattaacccgattagcggcggcaccaactataacgatcagtttaaaggc

SEQ ID NO: 52 - protein, 17 aa, synthetic construct
EINPISGGTNYNDQFKG

SEQ ID NO: 53 - DNA, 18 bp, synthetic construct
  source 1..18: mol_type = other DNA
agcgaatatctgattgat

SEQ ID NO: 54 - protein, 6 aa, synthetic construct
SEYLID

SEQ ID NO: 55 - DNA, 657 bp, synthetic construct
  source 1..657: mol_type = other DNA
gatattgaactgacccagagcccgctgaccctgagcgtgaccattggccagccggcgagcattagctgcaaaagcgaacagagcctgctggatagcgatagcaaaacctatctgaactggctgctgcagcgcccgggccagagcccgaaacgcctgatttatctggtgagcaaactggatagcggcgtgccggatcgctttaccggcagcggcagcggcaccgaatttaccctgaacattagccgcgtggaaaccgaagatctgggcgtgtattattgctggcagggcacccattttcgctggacctttggcggcggcaccaaactggaaattaaacgcgcagatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgt

SEQ ID NO: 56 - protein, 117 aa, synthetic construct
DIELTQSPLTLSVTIGQPASISCKSEQSLLDSDSKTYLNWLLQRPGQSPKRLIYLVSKLDSGVPDRFTGSGSGTEFTLNISRVETEDLGVYYCWQGTHFRWTFGGGTKLEIKRADAA

SEQ ID NO: 57 - DNA, 339 bp, synthetic construct
  source 1..339: mol_type = other DNA
gatattgaactgacccagagcccgctgaccctgagcgtgaccattggccagccggcgagcattagctgcaaaagcgaacagagcctgctggatagcgatagcaaaacctatctgaactggctgctgcagcgcccgggccagagcccgaaacgcctgatttatctggtgagcaaactggatagcggcgtgccggatcgctttaccggcagcggcagcggcaccgaatttaccctgaacattagccgcgtggaaaccgaagatctgggcgtgtattattgctggcagggcacccattttcgctggacctttggcggcggcaccaaactggaaattaaacgc

SEQ ID NO: 58 - protein, 219 aa, synthetic construct
DIELTQSPLTLSVTIGQPASISCKSEQSLLDSDSKTYLNWLLQRPGQSPKRLIYLVSKLDSGVPDRFTGSGSGTEFTLNISRVETEDLGVYYCWQGTHFRWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 59 - DNA, 48 bp, synthetic construct
  source 1..48: mol_type = other DNA
aaaagcgaacagagcctgctggatagcgatagcaaaacctatctgaac

SEQ ID NO: 60 - protein, 16 aa, synthetic construct
KSEQSLLDSDSKTYLN

SEQ ID NO: 61 - DNA, 21 bp, synthetic construct
  source 1..21: mol_type = other DNA
ctggtgagcaaactggatagc

SEQ ID NO: 62 - protein, 7 aa, synthetic construct
LVSKLDS

SEQ ID NO: 63 - DNA, 27 bp, synthetic construct
  source 1..27: mol_type = other DNA
tggcagggcacccattttcgctggacc

SEQ ID NO: 64 - protein, 9 aa, synthetic construct
WQGTHFRWT

SEQ ID NO: 65 - DNA, 1308 bp, synthetic construct
  source 1..1308: mol_type = other DNA
caggtgaaactgcagcagagcggcccggatctggtgaaaccgggcgcgagcgtgaaaattagctgcaaaaccagcggctatacctttaccgaatataccatgcattgggtgaaacagagccagggcaaaagcctggaatggattggcggcattaacccgaacaaccgcggcaccagctataaccagaaatttaaaggcaaagcgaccctgaccgtggataaaagcagcagcaccgcgtatatggatctgcgcagcctgaccagcgaagatagcgcggtgtattattgcgcgattagcagctggggccagggcaccaccgtgaccgtgagcagcagcgctaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaa

SEQ ID NO: 66 - protein, 436 aa, synthetic construct
QVKLQQSGPDLVKPGASVKISCKTSGYTFTEYTMHWVKQSQGKSLEWIGGINPNNRGTSYNQKFKGKATLTVDKSSSTAYMDLRSLTSEDSAVYYCAISSWGQGTTVTVSSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 67 - DNA, 333 bp, synthetic construct
  source 1..333: mol_type = other DNA
caggtgaaactgcagcagagcggcccggatctggtgaaaccgggcgcgagcgtgaaaattagctgcaaaaccagcggctatacctttaccgaatataccatgcattgggtgaaacagagccagggcaaaagcctggaatggattggcggcattaacccgaacaaccgcggcaccagctataaccagaaatttaaaggcaaagcgaccctgaccgtggataaaagcagcagcaccgcgtatatggatctgcgcagcctgaccagcgaagatagcgcggtgtattattgcgcgattagcagctggggccagggcaccaccgtgaccgtgagcagc

SEQ ID NO: 68 - protein, 111 aa, synthetic construct
QVKLQQSGPDLVKPGASVKISCKTSGYTFTEYTMHWVKQSQGKSLEWIGGINPNNRGTSYNQKFKGKATLTVDKSSSTAYMDLRSLTSEDSAVYYCAISSWGQGTTVTVSS

SEQ ID NO: 69 - DNA, 15 bp, synthetic construct
  source 1..15: mol_type = other DNA
gaatataccatgcat

SEQ ID NO: 70 - protein, 5 aa, synthetic construct
EYTMH

SEQ ID NO: 71 - DNA, 51 bp, synthetic construct
  source 1..51: mol_type = other DNA
ggcattaacccgaacaaccgcggcaccagctataaccagaaatttaaaggc

SEQ ID NO: 72 - protein, 17 aa, synthetic construct
GINPNNRGTSYNQKFKG

SEQ ID NO: 73 - DNA, 12 bp, synthetic construct
  source 1..12: mol_type = other DNA
gcgattagcagc

SEQ ID NO: 74 - protein, 4 aa, synthetic construct
AISS

SEQ ID NO: 75 - DNA, 657 bp, synthetic construct
  source 1..657: mol_type = other DNA
gatattgaactgacccagagcccgctgaccctgagcgtgattattggccagccggcgagcattagctgcaaaagcagccagagcctgctgcatagcgatggcaaaacctatctgaactggctgtttcagcgcccgggccagagcccgaaacgcctgatttatctggaaagcaaactggatagccgcgtgccggatcgctttaccggcagcggcagcggcaccgattttaccctgaaaattagccgcgtggaagcggaagatctgggcgtgtattattgctggcagggcacccgctttccgcatacctttggcggcggcacccgcctggaaattaaacgcgcagatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgt

SEQ ID NO: 76 - protein, 219 aa, synthetic construct
DIELTQSPLTLSVIIGQPASISCKSSQSLLHSDGKTYLNWLFQRPGQSPKRLIYLESKLDSRVPDRFTGSGSGTDFTLKISRVEAEDLGVYYCWQGTRFPHTFGGGTRLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 77 - DNA, 339 bp, synthetic construct
  source 1..339: mol_type = other DNA
gatattgaactgacccagagcccgctgaccctgagcgtgattattggccagccggcgagcattagctgcaaaagcagccagagcctgctgcatagcgatggcaaaacctatctgaactggctgtttcagcgcccgggccagagcccgaaacgcctgatttatctggaaagcaaactggatagccgcgtgccggatcgctttaccggcagcggcagcggcaccgattttaccctgaaaattagccgcgtggaagcggaagatctgggcgtgtattattgctggcagggcacccgctttccgcatacctttggcggcggcacccgcctggaaattaaacgc

SEQ ID NO: 78 - protein, 113 aa, synthetic construct
DIELTQSPLTLSVIIGQPASISCKSSQSLLHSDGKTYLNWLFQRPGQSPKRLIYLESKLDSRVPDRFTGSGSGTDFTLKISRVEAEDLGVYYCWQGTRFPHTFGGGTRLEIKR

SEQ ID NO: 79 - DNA, 48 bp, synthetic construct
  source 1..48: mol_type = other DNA
aaaagcagccagagcctgctgcatagcgatggcaaaacctatctgaac

SEQ ID NO: 80 - protein, 16 aa, synthetic construct
KSSQSLLHSDGKTYLN

SEQ ID NO: 81 - DNA, 21 bp, synthetic construct
  source 1..21: mol_type = other DNA
ctggaaagcaaactggatagc

SEQ ID NO: 82 - protein, 7 aa, synthetic construct
LESKLDS

SEQ ID NO: 83 - DNA, 27 bp, synthetic construct
  source 1..27: mol_type = other DNA
tggcagggcacccgctttccgcatacc

SEQ ID NO: 84 - protein, 9 aa, synthetic construct
WQGTRFPHT

SEQ ID NO: 85 - protein, 62 aa, synthetic construct
MNFGAFSINPAMMAAAQAALQSSWGMMGMLASQQNQSGPSGNNQNQGNMQMDSKSSGWGMNH